SEQUENCE LISTING (63 sequences)

SEQ ID NO: 1 - protein, 10 aa, Artificial Sequence
  Synthetic Construct
  VARIANT 6: Xaa = Asp or Gly
GFTFSXSWIH

SEQ ID NO: 2 - protein, 18 aa, Artificial Sequence
  Synthetic Construct
  VARIANT 4: Xaa = Ser or Leu
  VARIANT 10: Xaa = Thr or Ser
AWIXPYGGSXYYADSVKG

SEQ ID NO: 3 - protein, 9 aa, Artificial Sequence
  Synthetic Construct
RHWPGGFDY

SEQ ID NO: 4 - protein, 25 aa, Artificial Sequence [sequence omitted]

SEQ ID NO: 5 - protein, 13 aa, Artificial Sequence
  Synthetic Construct
WVRQAPGKGLEWV

SEQ ID NO: 6 - protein, 32 aa, Artificial Sequence
  Synthetic Construct
RFTISADTSKNTAYLQMNSLRAEDTAVYYCAR

SEQ ID NO: 7 - protein, 11 aa, Artificial Sequence
  Synthetic Construct
WGQGTLVTVSA

SEQ ID NO: 8 - protein, 11 aa, Artificial Sequence
  Synthetic Construct
  VARIANT 5: Xaa = Asp or Val
  VARIANT 6: Xaa = Val or Ile
  VARIANT 7: Xaa = Ser or Asn
  VARIANT 9: Xaa = Ala or Phe
  VARIANT 10: Xaa = Val or Leu
RASQXXXTXXA

SEQ ID NO: 9 - protein, 7 aa, Artificial Sequence
  Synthetic Construct
  VARIANT 4: Xaa = Phe or Thr
  VARIANT 6: Xaa = Tyr or Ala
SASXLXS

SEQ ID NO: 10 - protein, 9 aa, Artificial Sequence
  Synthetic Construct
  VARIANT 3: Xaa = Tyr, Gly, Phe, or Ser
  VARIANT 4: Xaa = Leu, Tyr, Phe or Trp
  VARIANT 5: Xaa = Tyr, Asn, Ala, Thr, Gly, Phe or Ile
  VARIANT 6: Xaa = His, Val, Pro, Thr or Ile
  VARIANT 8: Xaa = Ala, Trp, Arg, Pro or Thr
QQXXXXPXT

SEQ ID NO: 11 - protein, 23 aa, Artificial Sequence
  Synthetic Construct
DIQMTQSPSSLSASVGDRVTITC

SEQ ID NO: 12 - protein, 15 aa, Artificial Sequence
  Synthetic Construct
WYQQKPGKAPKLLIY

SEQ ID NO: 13 - protein, 32 aa, Artificial Sequence
  Synthetic Construct
GVPSRFSGSGSGTDFTLTISSLQPEDFATYYC

SEQ ID NO: 14 - protein, 11 aa, Artificial Sequence
  Synthetic Construct
FGQGTKVEIKR

SEQ ID NO: 15 - protein, 10 aa, Artificial Sequence
  Synthetic Construct
GFTFSDSWIH

SEQ ID NO: 16 - protein, 18 aa, Artificial Sequence
  Synthetic Construct
AWISPYGGSTYYADSVKG

SEQ ID NO: 17 - protein, 11 aa, Artificial Sequence
  Synthetic Construct
RASQDVSTAVA

SEQ ID NO: 18 - protein, 7 aa, Artificial Sequence
  Synthetic Construct
SASFLYS

SEQ ID NO: 19 - protein, 9 aa, Artificial Sequence
  Synthetic Construct
QQYLYHPAT

SEQ ID NO: 20 - protein, 118 aa, Artificial Sequence
  Synthetic Construct
EVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSA

SEQ ID NO: 21 - protein, 108 aa, Artificial Sequence
  Synthetic Construct
DIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKR

SEQ ID NO: 22 - protein, 440 aa, Artificial Sequence
  Synthetic Construct
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 23 - protein, 214 aa, Artificial Sequence
  Synthetic Construct
EIVLTQSPATLSLSPGERATLSCRASQSVSSYLAWYQQKPGQAPRLLIYDASNRATGIPARFSGSGSGTDFTLTISSLEPEDFAVYYCQQSSNWPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 24 - protein, 118 aa, Artificial Sequence [sequence omitted]

SEQ ID NO: 25 - protein, 11 aa, Artificial Sequence
  Synthetic Construct
WGQGTLVTVSS

SEQ ID NO: 26 - protein, 447 aa, Artificial Sequence
  Synthetic Construct
EVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 27 - protein, 214 aa, Artificial Sequence
  Synthetic Construct
DIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 28 - protein, 122 aa, Artificial Sequence
  Synthetic Construct
EVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSASTK

SEQ ID NO: 29 - protein, 108 aa, Artificial Sequence; identical to SEQ ID NO: 21
  Synthetic Construct

SEQ ID NO: 30 - protein, 126 aa, Artificial Sequence
  Synthetic Construct
QVQLVQSGAEVKKPGASVKVSCKASGYTFTSYDINWVRQATGQGLEWMGWLNPDSGVIRYAQKFQGRVTMTRDTSISTAYMELSSLRSEDTAVYYCAREWFGELPSYYFYSGMDVWGQGTTVTVSS

SEQ ID NO: 31 - protein, 107 aa, Artificial Sequence
  Synthetic Construct
EIVLTQSPATLSLSPGERATLSCRASQSVSSYLAWYQQKPGQAPRLLIYDASNRATGIPARFSGSGSGTDFTLTISSLEPEDFAVYYCQQRSNWPPTFGPGTKVDIK

SEQ ID NO: 32 - protein, 5 aa, Artificial Sequence
  Synthetic Construct
SYDIN

SEQ ID NO: 33 - protein, 17 aa, Artificial Sequence
  Synthetic Construct
WLNPDSGVIRYAQKFQG

SEQ ID NO: 34 - protein, 17 aa, Artificial Sequence
  Synthetic Construct
EWFGELPSYYFYSGMDV

SEQ ID NO: 35 - protein, 11 aa, Artificial Sequence
  Synthetic Construct
RASQSVSSYLA

SEQ ID NO: 36 - protein, 7 aa, Artificial Sequence
  Synthetic Construct
DASNRAT

SEQ ID NO: 37 - protein, 9 aa, Artificial Sequence
  Synthetic Construct
QQRSNWPPT

SEQ ID NO: 38 - protein, 123 aa, Artificial Sequence
  Synthetic Construct
QVQLVQSGAEVKKPGASVKVSCKAFAYTFSTYGISWVRQAPGQGLEWMGWISAYNSNTNYAQKVQGRITMTTDTSTRTAYMELRGLRSDDTAVYFCATFFGGHSGYHYGLDVWGQGTTVTVSS

SEQ ID NO: 39 - protein, 108 aa, Artificial Sequence
  Synthetic Construct
  VARIANT 5, 48: Xaa = Any Amino Acid
EIVLXQSPATLSLSPGERATLSCRASQSVSSYLAWYQQKPGQAPRLLXYDASNRATGIPARFSGSGSGTDFTLTISSLEPEDFAVYYCQQRSNWPPYTFGQGTKLEIK

SEQ ID NO: 40 - protein, 5 aa, Artificial Sequence
  Synthetic Construct
TYGIS

SEQ ID NO: 41 - protein, 17 aa, Artificial Sequence
  Synthetic Construct
WISAYNSNTNYAQKVQG

SEQ ID NO: 42 - protein, 14 aa, Artificial Sequence
  Synthetic Construct
FFGGHSGYHYGLDV

SEQ ID NO: 43 - protein, 11 aa, Artificial Sequence
  Synthetic Construct
RASQSVSSYLA

SEQ ID NO: 44 - protein, 7 aa, Artificial Sequence
  Synthetic Construct
DASNRAT

SEQ ID NO: 45 - protein, 10 aa, Artificial Sequence
  Synthetic Construct
QQRSNWPPYT

SEQ ID NO: 46 - protein, 123 aa, Artificial Sequence
  Synthetic Construct
EVQLVESGGGLVQPGRSLRLSCAASGFTFDDYAMHWVRQAPGKGLEWVSGINWSSGGIGYADSVKGRFTISRDNAKNSLYLQMNSLRAEDTALYYCARDIGGFGEFYWNFGLWGRGTLVTVSS

SEQ ID NO: 47 - protein, 108 aa, Artificial Sequence
  Synthetic Construct
EIVLTQSPATLSLSPGERATLSCRASQSVRSYLAWYQQKPGQAPRLLIYDASNRATGIPARFSGSGSGTDFTLTISSLEPEDFAVYYCQQRSNWPPATFGGGTKVEIK

SEQ ID NO: 48 - protein, 5 aa, Artificial Sequence
  Synthetic Construct
DYAMH

SEQ ID NO: 49 - protein, 17 aa, Artificial Sequence
  Synthetic Construct
GINWSSGGIGYADSVKG

SEQ ID NO: 50 - protein, 14 aa, Artificial Sequence
  Synthetic Construct
DIGGFGEFYWNFGL

SEQ ID NO: 51 - protein, 11 aa, Artificial Sequence
  Synthetic Construct
RASQSVRSYLA

SEQ ID NO: 52 - protein, 7 aa, Artificial Sequence
  Synthetic Construct
DASNRAT

SEQ ID NO: 53 - protein, 10 aa, Artificial Sequence
  Synthetic Construct
QQRSNWPPAT

SEQ ID NO: 54 - protein, 127 aa, Artificial Sequence
  Synthetic Construct
QVQLVQSGAEVKKPGSSVKVSCKASGGTLSSYAFSWVRQAPGQGLEWMGGIIPFFGTTNYAQKFQGRVIITADESTNTAYMELSGLRSEDTAVYYCARDRDYYGLGSPFYYYGMDVWGQGTTVTVSS

SEQ ID NO: 55 - protein, 107 aa, Artificial Sequence
  Synthetic Construct
EIVLTQSPDFQSVTPKEKVTITCRASQSIGSSLHWYQQKPDQSPKLLIKYASQSFSGVPSRFSGSGSGTDFTLTINSLEAEDAATYYCHQSSSLPWTFGQGTKVEIK

SEQ ID NO: 56 - protein, 5 aa, Artificial Sequence
  Synthetic Construct
SYAFS

SEQ ID NO: 57 - protein, 17 aa, Artificial Sequence
  Synthetic Construct
GIIPFFGTTNYAQKFQG

SEQ ID NO: 58 - protein, 18 aa, Artificial Sequence
  Synthetic Construct
DRDYYGLGSPFYYYGMDV

SEQ ID NO: 59 - protein, 11 aa, Artificial Sequence
  Synthetic Construct
RASQSIGSSLH

SEQ ID NO: 60 - protein, 7 aa, Artificial Sequence
  Synthetic Construct
YASQSFS

SEQ ID NO: 61 - protein, 9 aa, Artificial Sequence
  Synthetic Construct
HQSSSLPWT

SEQ ID NO: 62 - protein, 447 aa, Artificial Sequence
  Synthetic Construct
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 63 - protein, 218 aa, Artificial Sequence
  Synthetic Construct
EIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2017/0274073 A1
(43) Pub. Date: Sep. 28, 2017

(54) METHODS OF TREATING CANCER USING PD-1 AXIS BINDING ANTAGONISTS AND IL-17 BINDING ANTAGONISTS

(71) Applicant: Genentech, Inc., South San Francisco, CA (US)

(72) Inventors: Jane GROGAN, San Francisco, CA (US); Yuanyuan XIAO, South San Francisco, CA (US); Patrick CAPLAZI, South San Francisco, CA (US); Steve LIANOGLOU, South San Francisco, CA (US); Jason HACKNEY, San Carlos, CA (US); Eugene Yu-Chuan CHIANG, South San Francisco, CA (US)

(73) Assignee: Genentech, Inc., South San Francisco, CA (US)

(21) Appl. No.: 15/448,437

(22) Filed: Mar. 2, 2017

Related U.S. Application Data
(63) Continuation of application No. PCT/US2015/050051, filed on Sep. 14, 2015.
(60) Provisional application No. 62/050,745, filed on Sep. 15, 2014.

(51) Int. Cl. A61K 39/395; C07K 16/30; C07K 16/28

(52) U.S. Cl. CPC A61K 39/39558; A61K 47/48238; C07K 16/2827; C07K 16/3053; A61K 2039/505

(57) ABSTRACT

The present disclosure provides methods comprising administering to the individual an effective amount of a PD-1 axis binding antagonist and an IL-17 binding antagonist. Further provided are kits comprising a PD-1 axis binding antagonist, an IL-17 binding antagonist, or both, as well as instructions for use thereof.

### CROSS-REFERENCE TO RELATED APPLICATIONS

This application claims the priority benefit of U.S. Provisional Application No. 62/050,745, filed Sep. 15, 2014, which is hereby incorporated by reference in its entirety.

### SUBMISSION OF SEQUENCE LISTING ON ASCII TEXT FILE

The content of the following submission on ASCII text file is incorporated herein by reference in its entirety: a computer readable form (CRF) of the Sequence Listing (file name: 146392027140SeqList.txt, date recorded: Sep. 14, 2015, size: 41 KB).

### FIELD

The present disclosure relates to methods of treating cancer by administering a PD-1 axis binding antagonist and an IL-17 binding antagonist.

### BACKGROUND

The provision of two distinct signals to T-cells is a widely accepted model for lymphocyte activation of resting T lymphocytes by antigen-presenting cells (APCs). Lafferty et al, Aust. J. Exp. Biol. Med. Sci. 53: 27-42 (1975). This model further provides for the discrimination of self from non-self and immune tolerance. Bretscher et al, Science 169: 1042-1049 (1970); Bretscher, P. A., P.N.A.S. USA 96: 185-190 (1999); Jenkins et al, J. Exp. Med. 165: 302-319 (1987). The primary signal, or antigen specific signal, is transduced through the T-cell receptor (TCR) following recognition of foreign antigen peptide presented in the context of the major histocompatibility-complex (MHC). The second or co-stimulatory signal is delivered to T-cells by co-stimulatory molecules expressed on antigen-presenting cells (APCs), and induce T-cells to promote clonal expansion, cytokine secretion and effector function. Lenschow et al., Ann. Rev. Immunol. 14:233 (1996). In the absence of co-stimulation, T-cells can become refractory to antigen stimulation, do not mount an effective immune response, and further may result in exhaustion or tolerance to foreign antigens.

In the two-signal model T-cells receive both positive and negative secondary co-stimulatory signals. The regulation of such positive and negative signals is critical to maximize the host's protective immune responses, while maintaining immune tolerance and preventing autoimmunity. Negative secondary signals seem necessary for induction of T-cell tolerance, while positive signals promote T-cell activation. While the simple two-signal model still provides a valid explanation for naive lymphocytes, a host's immune response is a dynamic process, and co-stimulatory signals can also be provided to antigen-exposed T-cells. The mechanism of co-stimulation is of therapeutic interest because the manipulation of co-stimulatory signals has shown to provide a means to either enhance or terminate cell-based immune response. Recently, it has been discovered that T cell dysfunction or anergy occurs concurrently with an induced and sustained expression of the inhibitory receptor, programmed death 1 polypeptide (PD-1). As a result, therapeutic targeting of PD-1 and other molecules which signal through interactions with PD-1, such as programmed death ligand 1 (PDL1) and programmed death ligand 2 (PDL2) are an area of intense interest.

PDL1 is overexpressed in many cancers and is often associated with poor prognosis (Okazaki T et al., Intern. Immun 2007 19(7):813) (Thompson R H et al., Cancer Res 2006, 66(7):3381). Interestingly, the majority of tumor infiltrating T lymphocytes predominantly express PD-1, in contrast to T lymphocytes in normal tissues and peripheral blood T lymphocytes indicating that up-regulation of PD-1 on tumor-reactive T cells can contribute to impaired antitumor immune responses (Blood 2009 114(8):1537). This may be due to exploitation of PDL1 signaling mediated by PDL1 expressing tumor cells interacting with PD-1 expressing T cells to result in attenuation of T cell activation and evasion of immune surveillance (Sharpe et al., Nat Rev 2002) (Keir M E et al., 2008 Annu. Rev. Immunol. 26:677). Therefore, inhibition of the PDL1/PD-1 interaction may enhance CD8+ T cell-mediated killing of tumors.

IL-17 is a pro-inflammatory molecule that stimulates epithelial, endothelial and fibroblastic cells to produce other inflammatory cytokines and chemokines including IL-6, IL-8, G-CSF, and MCP-1 [see, Yao, Z. et al., J. Immunol., 122(12):5483-5486 (1995); Yao, Z. et al, Immunity, 3(6):811-821 (1995); Fossiez, F., et al., J. Exp. Med., 183(6): 2593-2603 (1996); Kennedy, J., et al., J. Interferon Cytokine Res., 16(8):611-7 (1996); Cai, X. Y., et al., Immunol. Lett, 62(1):51-8 (1998); Jovanovic, D. V., et al., J. Immunol., 160(7):3513-21 (1998); Laan, M., et al., J. Immunol., 162(4):2347-52 (1999); Linden, A., et al., Eur Respir J, 15(5):973-7 (2000); and Aggarwal, S. and Gurney, A. L., J Leukoc Biol. 71(1):1-8 (2002)]. IL-17 also synergizes with other cytokines including TNF-α, and IL-1β to further induce chemokine expression (Chabaud, M., et al., J. Immunol. 161(1):409-14 (1998)). Interleukin 17 (IL-17) exhibits pleitropic biological activities on various types of cells. IL-17 also has the ability to induce ICAM-1 surface expression, proliferation of T cells, and growth and differentiation of CD34⁺ human progenitors into neutrophils.

There remains a need for such an optimal therapy for treating, stabilizing, preventing, and/or delaying development of various cancers.

All references cited herein, including patent applications, patent publications, and UniProtKB/Swiss-Prot Accession numbers are herein incorporated by reference in their entirety, as if each individual reference were specifically and individually indicated to be incorporated by reference.

### BRIEF SUMMARY

The present disclosure describes a combination treatment comprising an effective amount of a PD-1 axis binding antagonist and an IL-17 binding antagonist.

In certain aspects, the present disclosure provides a method for treating or delaying progression of cancer in an individual comprising administering to the individual an effective amount of a PD-1 axis binding antagonist and an IL-17 binding antagonist. In another aspect, the present disclosure provides a method of enhancing immune function in an individual having cancer comprising administering an effective amount of a combination of a PD-1 axis binding antagonist and an IL-17 binding antagonist.

In another aspect, the present disclosure provides a method for identifying an individual with cancer for treatment with a PD-1 axis binding antagonist and an IL-17 binding antagonist, the method comprising: (a) detecting expression of IL-17 in a biopsy sample obtained from the cancer in the individual; and (b) if the biopsy sample shows expression of IL-17, or if the biopsy sample shows increased expression of IL-17 as compared to a reference or a reference sample, administering to the individual an effective amount of a PD-1 axis binding antagonist and an IL-17 binding antagonist. In another aspect, the present disclosure provides a method for identifying an individual with cancer for treatment with a PD-1 axis binding antagonist and an IL-17 binding antagonist, the method comprising: (a) detecting expression of an IL-17 gene signature (such as one or more genes selected from IL-17A, IL-17F, IL-8, CSF3, CXCL1, CXCL3, and CCL20) in a biopsy sample obtained from the cancer in the individual; and (b) if the biopsy sample shows expression of the IL-17 gene signature, or if the biopsy sample shows increased expression of the IL-17 gene signature as compared to a reference or a reference sample, administering to the individual an effective amount of a PD-1 axis binding antagonist and an IL-17 binding antagonist. In another aspect, the present disclosure provides a method for identifying an individual with cancer for treatment with a PD-1 axis binding antagonist and an IL-17 binding antagonist, the method comprising: (a) detecting expression of an IL-17 gene signature (such as one or more genes selected from CD4, CD8a, IL17A, IL17B, IL17C, IL17D, IL17F, IL17RA, IL17RC, C3, CCL2, CCL20, CSF2, CSF3, CXCL1, CXCL2, CXCL3, CXCL5, CXCL10, CXCR1, CXCR2, ICAM1, IL6, IL8, MMP1, MMP2, MMP3, MMP8, MMP9, MMP13, MMP14, MMP25, NCF4, NFKBIZ, S100A8, S100A9, SAA2, SAA1, SAA3, SAA4, TIMP1, TIMP2, TIMP3, and TIMP4) in a biopsy sample obtained from the cancer in the individual; and (b) if the biopsy sample shows expression of the IL-17 gene signature, or if the biopsy sample shows increased expression of the IL-17 gene signature as compared to a reference or a reference sample, administering to the individual an effective amount of a PD-1 axis binding antagonist and an IL-17 binding antagonist. In another aspect, the present disclosure provides a method for identifying an individual with cancer for treatment with a PD-1 axis binding antagonist and an IL-17 binding antagonist, the method comprising detecting expression of an IL-17 gene signature (such as one or more genes selected from CD4, CD8a, IL17A, IL17B, IL17C, IL17D, IL17F, IL17RA, IL17RC, C3, CCL2, CCL20, CSF2, CSF3, CXCL1, CXCL2, CXCL3, CXCL5, CXCL10, CXCR1, CXCR2, ICAM1, IL6, IL8, MMP1, MMP2, MMP3, MMP8, MMP9, MMP13, MMP14, MMP25, NCF4, NFKBIZ, S100A8, S100A9, SAA2, SAA1, SAA3, SAA4, TIMP1, TIMP2, TIMP3, and TIMP4) in a biopsy sample obtained from the cancer in the individual, wherein the individual is identified for the treatment if the biopsy sample shows expression of the IL-17 gene signature, or if the biopsy sample shows increased expression of the IL-17 gene signature as compared to a reference or a reference sample.

In some embodiments, the PD-1 axis binding antagonist is selected from the group consisting of a PD-1 binding antagonist, a PDL1 binding antagonist and a PDL2 binding antagonist.

In some embodiments, the PD-1 axis binding antagonist is a PD-1 binding antagonist. In some embodiments, the PD-1 binding antagonist inhibits the binding of PD-1 to its ligand binding partners. In some embodiments, the PD-1 binding antagonist inhibits the binding of PD-1 to PDL1. In some embodiments, the PD-1 binding antagonist inhibits the binding of PD-1 to PDL2. In some embodiments, the PD-1 binding antagonist inhibits the binding of PD-1 to both PDL1 and PDL2. In some embodiments, PD-1 binding antagonist is an antibody. In some embodiments, the anti-PD-1 antibody is a monoclonal antibody. In some embodiments, the anti-PD-1 antibody is an antibody fragment selected from the group consisting of Fab, Fab′-SH, Fv, scFv, and (Fab′)₂ fragments. In some embodiments, PD-1 binding antagonist is nivolumab, pembrolizumab, CT-011, or AMP-224.

In some embodiments, the PD-1 axis binding antagonist is a PDL1 binding antagonist. In some embodiments, the PDL1 binding antagonist inhibits the binding of PDL1 to PD-1. In some embodiments, the PDL1 binding antagonist inhibits the binding of PDL1 to B7-1. In some embodiments, the PDL1 binding antagonist inhibits the binding of PDL1 to both PD-1 and B7-1. In some embodiments, the PDL1 binding antagonist is an anti-PDL1 antibody. In some embodiments, the anti-PDL1 antibody is a monoclonal antibody. In some embodiments, the anti-PDL1 antibody is an antibody fragment selected from the group consisting of Fab, Fab′-SH, Fv, scFv, and (Fab′)₂ fragments. In some embodiments, the anti-PDL1 antibody is a humanized antibody or a human antibody. In some embodiments, the PDL1 binding antagonist is selected from the group consisting of: YW243.55.S70, MPDL3280A, MDX-1105, and MEDI4736.

In some embodiments, the anti-PDL1 antibody comprises a heavy chain comprising HVR-H1 sequence of SEQ ID NO:15, HVR-H2 sequence of SEQ ID NO:16, and HVR-H3 sequence of SEQ ID NO:3; and a light chain comprising HVR-L1 sequence of SEQ ID NO:17, HVR-L2 sequence of SEQ ID NO:18, and HVR-L3 sequence of SEQ ID NO:19. In some embodiments, anti-PDL1 antibody comprises a heavy chain variable region comprising the amino acid sequence of SEQ ID NO:24 or SEQ ID NO:28 and a light chain variable region comprising the amino acid sequence of SEQ ID NO:21. In some embodiments, the anti-PDL1 antibody comprises a heavy chain comprising the amino acid sequence of SEQ ID NO:26 and/or a light chain comprising the amino acid sequence of SEQ ID NO:27.

In some embodiments, the PD-1 axis binding antagonist is a PDL2 binding antagonist. In some embodiments, PDL2 binding antagonist is an antibody. In some embodiments, the anti-PDL2 antibody is a monoclonal antibody. In some embodiments, the anti-PDL2 antibody is an antibody fragment selected from the group consisting of Fab, Fab′-SH, Fv, scFv, and (Fab′)₂ fragments. In some embodiments, PDL2 binding antagonist is an immunoadhesin.

In some embodiments, the IL-17 binding antagonist inhibits the binding of IL-17 to the IL-17 receptor. In some embodiments, the IL-17 binding antagonist is an antibody. In some embodiments, the IL-17 binding antagonist is a monoclonal antibody. In some embodiments, the IL-17 binding antagonist is an antibody fragment selected from the group consisting of Fab, Fab′-SH, Fv, scFv, and (Fab′)₂ fragments. In some embodiments, the IL-17 binding antagonist is a humanized antibody or a human antibody.

In some embodiments, the anti-IL-17 antibody comprises a heavy chain comprising CDR-H1 sequence of SEQ ID NO:32, CDR-H2 sequence of SEQ ID NO:33, and CDR-H3 sequence of SEQ ID NO:34; and a light chain comprising CDR-L1 sequence of SEQ ID NO:35, CDR-L2 sequence of SEQ ID NO:36, and CDR-L3 sequence of SEQ ID NO:37. In some embodiments, the anti-IL-17 antibody comprises a heavy chain variable region comprising the amino acid sequence of SEQ ID NO:30 and a light chain variable region comprising the amino acid sequence of SEQ ID NO:31.

In some embodiments, the anti-IL-17 antibody comprises a heavy chain comprising CDR-H1 sequence of SEQ ID NO:40, CDR-H2 sequence of SEQ ID NO:41, and CDR-H3 sequence of SEQ ID NO:42; and a light chain comprising CDR-L1 sequence of SEQ ID NO:43, CDR-L2 sequence of SEQ ID NO:44, and CDR-L3 sequence of SEQ ID NO:45. In some embodiments, the anti-IL-17 antibody comprises a heavy chain variable region comprising the amino acid sequence of SEQ ID NO:38 and a light chain variable region comprising the amino acid sequence of SEQ ID NO:39.

In some embodiments, the anti-IL-17 antibody comprises a heavy chain comprising CDR-H1 sequence of SEQ ID NO:48, CDR-H2 sequence of SEQ ID NO:49, and CDR-H3 sequence of SEQ ID NO:50; and a light chain comprising CDR-L1 sequence of SEQ ID NO:51, CDR-L2 sequence of SEQ ID NO:52, and CDR-L3 sequence of SEQ ID NO:53. In some embodiments, the anti-IL-17 antibody comprises a heavy chain variable region comprising the amino acid sequence of SEQ ID NO:46 and a light chain variable region comprising the amino acid sequence of SEQ ID NO:47.

In some embodiments, the anti-IL-17 antibody comprises a heavy chain comprising CDR-H1 sequence of SEQ ID NO:56, CDR-H2 sequence of SEQ ID NO:57, and CDR-H3 sequence of SEQ ID NO:58; and a light chain comprising CDR-L1 sequence of SEQ ID NO:59, CDR-L2 sequence of SEQ ID NO:60, and CDR-L3 sequence of SEQ ID NO:61. In some embodiments, the anti-IL-17 antibody comprises a heavy chain variable region comprising the amino acid sequence of SEQ ID NO:54 and a light chain variable region comprising the amino acid sequence of SEQ ID NO:55.

In some embodiments, the IL-17 binding antagonist is an anti-IL-17 antibody. In some embodiments, the anti-IL-17 antibody specifically binds to IL-17A. In some embodiments, the anti-IL-17 antibody specifically binds to IL-17F. In some embodiments, the anti-IL-17 antibody specifically binds to IL-17A and IL-17F. In some embodiments, the anti-IL-17 antibody is ixekizumab, bimekizumab, or secukinumab.

In some embodiments, the IL-17 binding antagonist is an anti-IL-17 receptor antibody. In some embodiments, the anti-IL-17 receptor antibody is brodalumab.

In some embodiments, the IL-17 binding antagonist is a soluble polypeptide comprising at least one exon from an IL-17 receptor. In some embodiments, the soluble polypeptide comprises at least one exon from IL-17RA and at least one exon from IL-17RC.

In some embodiments, the method further comprises a step of detecting biomarker expressions in a biopsy sample from the cancer of the individual before or after administering the PD-1 axis binding antagonist and the IL-17 binding antagonist. In some embodiments, a biopsy sample obtained from the cancer of the individual shows expression of IL-17. In some embodiments, the expression of IL-17 is expression of IL-17 mRNA. In some embodiments, the expression of IL-17 is expression of IL-17 protein. In some embodiments, the biopsy sample obtained from the cancer shows elevated expression of IL-17 as compared to a reference or a reference sample. In some embodiments, a biopsy sample obtained from the cancer of the individual shows expression of one or more genes selected from the group consisting of IL-17A, IL-17F, IL-8, CSF3, CXCL1, CXCL3, and CCL20. In some embodiments, the biopsy sample obtained from the cancer shows elevated expression of one or more genes selected from the group consisting of IL-17A, IL-17F, IL-8, CSF3, CXCL1, CXCL3, and CCL20 as compared to a reference or a reference sample. In some embodiments, the cancer is selected from the group consisting of renal cell carcinoma, bladder cancer, non-small-cell lung cancer, squamous non-small-cell lung cancer, non-squamous non-small-cell lung cancer, colorectal cancer, melanoma, ovarian cancer, breast cancer, hormone receptor-positive breast cancer, HER2-positive breast cancer, and triple-negative breast cancer. In some embodiments, a biopsy sample obtained from the cancer of the individual shows expression of one or more genes selected from the group consisting of CD4, CD8a, IL17A, IL17B, IL17C, IL17D, IL17F, IL17RA, IL17RC, C3, CCL2, CCL20, CSF2, CSF3, CXCL1, CXCL2, CXCL3, CXCL5, CXCL10, CXCR1, CXCR2, ICAM1, IL6, IL8, MMP1, MMP2, MMP3, MMP8, MMP9, MMP13, MMP14, MMP25, NCF4, NFKBIZ, S100A8, S100A9, SAA2, SAA1, SAA3, SAA4, TIMP1, TIMP2, TIMP3, and TIMP4. In some embodiments, a biopsy sample obtained from the cancer of the individual shows expression of at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, or at least 44 genes selected from CD4, CD8a, IL17A, IL17B, IL17C, IL17D, IL17F, IL17RA, IL17RC, C3, CCL2, CCL20, CSF2, CSF3, CXCL1, CXCL2, CXCL3, CXCL5, CXCL10, CXCR1, CXCR2, ICAM1, IL6, IL8, MMP1, MMP2, MMP3, MMP8, MMP9, MMP13, MMP14, MMP25, NCF4, NFKBIZ, S100A8, S100A9, SAA2, SAA1, SAA3, SAA4, TIMP1, TIMP2, TIMP3, and TIMP4. In some embodiments, the biopsy sample obtained from the cancer shows elevated expression of one or more genes selected from the group consisting of CD4, CD8a, IL17A, IL17B, IL17C, IL17D, IL17F, IL17RA, IL17RC, C3, CCL2, CCL20, CSF2, CSF3, CXCL1, CXCL2, CXCL3, CXCL5, CXCL10, CXCR1, CXCR2, ICAM1, IL6, IL8, MMP1, MMP2, MMP3, MMP8, MMP9, MMP13, MMP14, MMP25, NCF4, NFKBIZ, S100A8, S100A9, SAA2, SAA1, SAA3, SAA4, TIMP1, TIMP2, TIMP3, and TIMP4 as compared to a reference or a reference sample. In some embodiments, the biopsy sample obtained from the cancer shows elevated expression of at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, or at least 44 genes selected from CD4, CD8a, IL17A, IL17B, IL17C, IL17D, IL17F, IL17RA, IL17RC, C3, CCL2, CCL20, CSF2, CSF3, CXCL1, CXCL2, CXCL3, CXCL5, CXCL10, CXCR1, CXCR2, ICAM1, IL6, IL8, MMP1, MMP2, MMP3, MMP8, MMP9, MMP13, MMP14, MMP25, NCF4, NFKBIZ, S100A8, S100A9, SAA2, SAA1, SAA3, SAA4, TIMP1, TIMP2, TIMP3, and TIMP4 as compared to a reference or a reference sample. In some embodiments, a biopsy sample obtained from the cancer of the individual shows expression of one or more genes selected from the group consisting of NFKBIZ, S100A8, and S100A9. In some embodiments, the biopsy sample obtained from the cancer shows elevated expression of one or more genes selected from the group consisting of NFKBIZ, S100A8, and S100A9 as compared to a reference or a reference sample.

In some embodiments, the treatment results in a sustained response in the individual after cessation of the treatment.

In some embodiments, the IL-17 binding antagonist and/or the PD-1 axis binding antagonist is administered continuously or intermittently. In some embodiments, the IL-17 binding antagonist is administered before the PD-1 axis binding antagonist. In some embodiments, the IL-17 binding antagonist is administered simultaneous with the PD-1 axis binding antagonist. In some embodiments, the IL-17 binding antagonist and the PD-1 axis binding antagonist are formulated in the same composition. In some embodiments, the IL-17 binding antagonist is administered after the PD-1 axis binding antagonist. In some embodiments, the PD-1 axis binding antagonist or the IL-17 binding antagonist is administered intravenously, intramuscularly, subcutaneously, topically, orally, transdermally, intraperitoneally, intraorbitally, by implantation, by inhalation, intrathecally, intraventricularly, or intranasally.

In another aspect, the present disclosure provides a kit comprising a PD-1 axis binding antagonist and a package insert comprising instructions for using the PD-1 axis binding antagonist in combination with an IL-17 binding antagonist to treat or delay progression of cancer in an individual. In another aspect, the present disclosure provides a kit comprising a PD-1 axis binding antagonist and an IL-17 binding antagonist, and a package insert comprising instructions for using the PD-1 axis binding antagonist and the IL-17 binding antagonist to treat or delay progression of cancer in an individual. In some embodiments, the PD-1 axis binding antagonist and the IL-17 binding antagonist are formulated in the same composition. In another aspect, the present disclosure provides a kit comprising an IL-17 binding antagonist and a package insert comprising instructions for using the IL-17 binding antagonist in combination with a PD-1 axis binding antagonist to treat or delay progression of cancer in an individual. In another aspect, the present disclosure provides a kit comprising a PD-1 axis binding antagonist and a package insert comprising instructions for using the PD-1 axis binding antagonist in combination with an IL-17 binding antagonist to enhance immune function in an individual having cancer. In another aspect, the present disclosure provides a kit comprising a PD-1 axis binding antagonist and an IL-17 binding antagonist, and a package insert comprising instructions for using the PD-1 axis binding antagonist and the IL-17 binding antagonist to enhance immune function in an individual having cancer. In some embodiments, the PD-1 axis binding antagonist and the IL-17 binding antagonist are formulated in the same composition. In another aspect, the present disclosure provides a kit comprising an IL-17 binding antagonist and a package insert comprising instructions for using the IL-17 binding antagonist in combination with a PD-1 axis binding antagonist to enhance immune function in an individual having cancer.

In another aspect, the present disclosure provides a method for treating or delaying progression of cancer in an individual comprising administering to the individual an effective amount of a multispecific (e.g., bispecific) antibody, wherein the multispecific antibody comprises: (a) a first binding specificity for PD-1, PDL1, and/or PDL2; and (b) a second binding specificity for IL-17 and/or IL-17R.

It is to be understood that one, some, or all of the properties of the various embodiments described above and herein may be combined to form other embodiments of the present invention. These and other aspects of the invention will become apparent to one of skill in the art. These and other embodiments of the invention are further described by the detailed description that follows.

### BRIEF DESCRIPTION OF THE DRAWINGS

FIGS. 1A-1D show the relative prevalence of IL-17A and IL-17F in samples representing multiple types of cancer. Each graph depicts the relative prevalence of each IL-17 expression state (as a fraction of 100%, or 1.0) in a set of samples (as well as the number of samples used, N). IL-17 expression states are: IL-17A/IL-17F double negative (“A−F−”), IL-17F positive and IL-17A negative (“F+ only”), IL-17A positive and IL-17F negative (“A+ only”), and IL-17A/IL-17F double positive (“A+F+”). Shown is the prevalence of each IL-17 expression state in colorectal cancer (“CRC,” FIG. 1A), hormone receptor-positive breast cancer (“HR+BC;” FIG. 1B), non-squamous non-small-cell lung cancer (“nonSquam-NSCLC,” FIG. 1C), and squamous non-small-cell lung cancer (“Squam-NSCLC,” FIG. 1D).

FIGS. 2A-2D show the relative prevalence of IL-17A and IL-17F in samples representing multiple types of cancer. Each graph depicts the relative prevalence of each IL-17 expression state (as a fraction of 100%, or 1.0) in a set of samples (as well as the number of samples used, N). IL-17 expression states are: IL-17A/IL-17F double negative (“A−F−”), IL-17F positive and IL-17A negative (“F+ only”), IL-17A positive and IL-17F negative (“A+ only”), and IL-17A/IL-17F double positive (“A+F+”). Shown is the prevalence of each IL-17 condition in triple negative breast cancer (“TNBC,” FIG. 2A), HER2-positive breast cancer (“HER2+BC,” FIG. 2B), renal cell carcinoma (“RCC,” FIG. 2C), and melanoma (FIG. 2D).

FIGS. 3A & 3B show the relative prevalence of IL-17A and IL-17F in samples representing multiple types of cancer. Each graph depicts the relative prevalence of each IL-17 expression state (as a fraction of 100%, or 1.0) in a set of samples (as well as the number of samples used, N). IL-17 conditions are: IL-17A/IL-17F double negative (“A−F−”), IL-17F positive and IL-17A negative (“F+ only”), IL-17A positive and IL-17F negative (“A+ only”), and IL-17A/IL-17F double positive (“A+F+”). Shown is the prevalence of each IL-17 condition in ovarian cancer (“OVA,” FIG. 3A) and bladder cancer (FIG. 3B).

FIG. 4 shows the association between IL-17 expression and response to anti-PDL1 treatment in melanoma patients. For each IL-17 condition, the percentage of samples showing the presence of IL-17 (determined as having a raw Ct of less than 30 cycles) and the number of samples (N) are depicted. IL-17 conditions are: ITT, intent-to-treat (all efficacy patients); BP, biomarker available patients; A+, IL-17A present (agnostic as to IL-17F presence); F+, IL-17F present (agnostic as to IL-17A presence); A+F+, IL-17A and IL-17F present; and A−F−, neither IL-17A nor IL-17F present.

FIGS. 5A-5D show the associations between the response to anti-PDL1 treatment in melanoma patients and IL-17A expression (FIG. 5A), IL-17F expression (FIG. 5B), IL-8 expression (FIG. 5C), and the average expression of all three genes (normalized to an average value of 0 and standard deviation of 1) (FIG. 5D).

FIGS. 6A-6D show the associations between the response to anti-PDL1 treatment in melanoma patients with an IHCIC score of 2+ and IL-17A expression (FIG. 6A), IL-17F expression (FIG. 6B), IL-8 expression (FIG. 6C), and the average expression of all three genes (normalized to an average value of 0 and standard deviation of 1) (FIG. 6D).

FIG. 7 shows the ROC (receiver-operating characteristic) analysis of IL-17 gene expression and response to anti-PDL1 treatment in melanoma patients by plotting sensitivity vs. 1-specificity. Area-under-the-curve (AUC) values are as depicted. Solid blue line depicts comparison of patients with complete or partial response to patients with stable or progressive disease. Dotted black line depicts comparison of patients with complete response, partial response, or stable disease to patients with progressive disease. Solid black line on diagonal shows the line of no-discrimination.

FIG. 8 shows the association between IL-17 expression and response to anti-PDL1 treatment in renal cell carcinoma patients. For each IL-17 condition, the percentage of samples showing the presence of IL-17 (determined as having a raw Ct of less than 30 cycles) and the number of samples (N) are depicted. IL-17 conditions are as described above for FIG. 4.

FIGS. 9A-9D show the associations between the response to anti-PDL1 treatment in renal cell carcinoma patients and IL-17A expression (FIG. 9A), IL-17F expression (FIG. 9B), IL-8 expression (FIG. 9C), and the average expression of all three genes (normalized to an average value of 0 and standard deviation of 1) (FIG. 9D).

FIGS. 10A-10D show the associations between the response to anti-PDL1 treatment in renal cell carcinoma patients with an IHCIC score of 2+ and IL-17A expression (FIG. 10A), IL-17F expression (FIG. 10B), IL-8 expression (FIG. 10C), and the average expression of all three genes (normalized to an average value of 0 and standard deviation of 1) (FIG. 10D).

FIG. 11 shows the ROC analysis of IL-17 gene expression and response to anti-PDL1 treatment in renal cell carcinoma patients by plotting sensitivity vs. 1-specificity. Area-under-the-curve (AUC) values are as depicted. Solid blue line depicts comparison of patients with complete or partial response to patients with stable or progressive disease. Dotted black line depicts comparison of patients with complete response, partial response, or stable disease to patients with progressive disease. Solid black line on diagonal shows the line of no-discrimination.

FIG. 12 shows the association between IL-17 expression and response to anti-PDL1 treatment in bladder cancer patients. For each IL-17 condition, the percentage of samples showing the presence of IL-17 (determined as having a raw Ct of less than 30 cycles) and the number of samples (N) are depicted. IL-17 conditions are as described above for FIG. 4.

FIGS. 13A-13D show the associations between the response to anti-PDL1 treatment in bladder cancer patients and IL-17A expression (FIG. 13A), IL-17F expression (FIG. 13B), IL-8 expression (FIG. 13C), and the average expression of all three genes (normalized to an average value of 0 and standard deviation of 1) (FIG. 13D).

FIGS. 14A-14D show the associations between the response to anti-PDL1 treatment in bladder cancer patients with an IHCIC score of 2+ and IL-17A expression (FIG. 14A), IL-17F expression (FIG. 14B), IL-8 expression (FIG. 14C), and the average expression of all three genes (normalized to an average value of 0 and standard deviation of 1) (FIG. 14D).

FIG. 15 shows the ROC analysis of IL-17 gene expression and response to anti-PDL1 treatment in bladder cancer patients by plotting sensitivity vs. 1-specificity. Area-under-the-curve (AUC) values are as depicted. Solid blue line depicts comparison of patients with complete or partial response to patients with stable or progressive disease. Dotted black line depicts comparison of patients with complete response, partial response, or stable disease to patients with progressive disease. Solid black line on diagonal shows the line of no-discrimination.

FIG. 16 shows the association between IL-17 expression and response to anti-PDL1 treatment in non-small-cell lung cancer patients. For each IL-17 condition, the percentage of samples showing the presence of IL-17 (determined as having a raw Ct of less than 30 cycles) and the number of samples (N) are depicted. IL-17 conditions are as described above for FIG. 4.

FIGS. 17A-17D show the associations between the response to anti-PDL1 treatment in non-small-cell lung cancer patients and IL-17A expression (FIG. 17A), IL-17F expression (FIG. 17B), IL-8 expression (FIG. 17C), and the average expression of all three genes (normalized to an average value of 0 and standard deviation of 1) (FIG. 17D).

FIGS. 18A-18D show the associations between the response to anti-PDL1 treatment in non-small-cell lung cancer patients with an IHCIC score of 2+ and IL-17A expression (FIG. 18A), IL-17F expression (FIG. 18B), IL-8 expression (FIG. 18C), and the average expression of all three genes (normalized to an average value of 0 and standard deviation of 1) (FIG. 18D).

FIG. 19 shows the ROC analysis of IL-17 gene expression and response to anti-PDL1 treatment in non-small-cell lung cancer patients by plotting sensitivity vs. 1-specificity. Area-under-the-curve (AUC) values are as depicted. Solid blue line depicts comparison of patients with complete or partial response to patients with stable or progressive disease. Dotted black line depicts comparison of patients with complete response, partial response, or stable disease to patients with progressive disease. Solid black line on diagonal shows the line of no-discrimination.

FIGS. 20A-20H show the associations between the response to anti-PDL1 treatment in non-small-cell lung cancer patients and IL-17A expression (FIG. 20A), IL-17F expression (FIG. 20B), IL-8 expression (FIG. 20C), CSF3 expression (FIG. 20D), CXCL1 expression (FIG. 20E), CXCL3 expression (FIG. 20F), CCL20 expression (FIG. 20G), and the average expression of all seven genes in the gene signature (normalized to an average value of 0 and standard deviation of 1) (FIG. 20H).

FIGS. 21A-21H show the associations between the response to anti-PDL1 treatment in non-small-cell lung cancer patients with an IHCIC score of 2+ and IL-17A expression (FIG. 21A), IL-17F expression (FIG. 21B), IL-8 expression (FIG. 21C), CSF3 expression (FIG. 21D), CXCL1 expression (FIG. 21E), CXCL3 expression (FIG. 21F), CCL20 expression (FIG. 21G), and the average expression of all seven genes in the gene signature (normalized to an average value of 0 and standard deviation of 1) (FIG. 21H).

FIG. 22 shows the ROC analysis of IL-17 gene signature expression and response to anti-PDL1 treatment in non-small-cell lung cancer patients by plotting sensitivity vs. 1-specificity. Area-under-the-curve (AUC) values are as depicted. Solid blue line depicts comparison of patients with complete or partial response to patients with stable or progressive disease. Dotted black line depicts comparison of patients with complete response, partial response, or stable disease to patients with progressive disease. Solid black line on diagonal shows the line of no-discrimination.

FIGS. 23A & 23B show the relative expression of Th17 (FIG. 23A) and T effector (Teff) (FIG. 23B) gene signatures in various cancer types, as labeled. The Th17 signature includes expression of IL17A, IL17F and RORC and the Teff signature includes expression of CD8, IFNgamma, granzyme A, granzyme B and peforin. Cycle threshold (Ct) values were normalized and converted to relative expression values (negative delta Ct) by subtracting the median gene expression estimated using all 96 genes on the array.

FIGS. 24A-24C show the relative expression of IL-17A (FIG. 24A), IL-17F (FIG. 24B), and IL-17A and IL-17F (FIG. 24C) gene signatures in various cancer types, as labeled. Cycle threshold (Ct) values were normalized and converted to relative expression values (negative delta Ct) by subtracting the median gene expression estimated using all 96 genes on the array.

FIG. 25 shows the relative expression of IL-17A in patients with melanoma, bladder cancer, and renal cancer showing responsiveness (PR, partial response; CR, complete response) or non-responsiveness (PD, progressive disease) to anti-PDL1 treatment. Number of samples for each cancer type is indicated (N). Samples were run in triplicate and cycle threshold (Ct) values were converted to relative expression values (negative delta Ct) by subtracting the mean of the five reference genes (SP2, GUSB, TMEM55B, VPS33B and SDHA) from the mean of each target gene.

FIGS. 26A & 26B show the relative expression of PDL1 (FIG. 26A) and IL-17F (FIG. 26B) in either responding patients (R) or non-responding patients (nR) with renal cell carcinoma, as labeled. This study included 8 responders and 5 non-responders. Samples were run in triplicate and cycle threshold (Ct) values were converted to relative expression values (negative delta Ct) by subtracting the mean of the five reference genes (SP2, GUSB, TMEM55B, VPS33B and SDHA) from the mean of each target gene.

FIG. 27A shows the relative expression of IL-17F in either responding patients (PR/CR) or non-responding patients (PD) with renal cell carcinoma, as labeled. This study included 2 responders and 5 non-responders. Samples were run in triplicate and cycle threshold (Ct) values were converted to relative expression values (negative delta Ct) by subtracting the mean of the five reference genes (SP2, GUSB, TMEM55B, VPS33B and SDHA) from the mean of each target gene.

FIG. 27B shows the relative expression of IL-17F in either early-responding patients or late-responding patients (greater than 6 months) with renal cell carcinoma, non-small-cell lung cancer, or melanoma, as labeled. This study included 14 early-responders and 11 late-responders. Samples were run in triplicate and cycle threshold (Ct) values were converted to relative expression values (negative delta Ct) by subtracting the mean of the five reference genes (SP2, GUSB, TMEM55B, VPS33B and SDHA) from the mean of each target gene.

FIGS. 28A-28D show several examples of IL-17A protein expression in non-small-cell lung cancer tissue. Scale of each immunohistochemical image is indicated by the scale bar.

FIGS. 29A-29C show several examples of IL-17A protein expression in colorectal cancer tissue. Scale of each immunohistochemical image is indicated by the scale bar.

FIG. 30 shows tumor volume over time in a mouse EMT6 breast carcinoma model receiving control, anti-PDL1, anti-IL-17, or anti-PDL1 and anti-IL-17 treatment, as labeled.

FIG. 31 shows cumulative gene expression of IL-17 inducible genes in various mouse tumors. RNA-Seq was performed to determine gene expression for each tumor sample. Gene expression was reported as reads per kilobase per million mapped reads (RPKM), and the sum of RPKM values for all genes was plotted as barplots using ExpressionPlot version 3.7.0 for data analysis.

FIGS. 32A-32W and 33A-33T show the relative expression of genes comprising an IL-17 inducible gene signature in Lewis lung carcinoma and B16.F10 melanoma orthotopic lung tumors. Each graph depicts the expression of the indicated gene relative to housekeeping gene expression.

FIGS. 34A-34W and 35A-35T show the relative expression of genes comprising an IL-17 inducible gene signature in Lewis lung carcinoma orthotopic lung tumors in syngeneic mice treated with anti-IL-17 antibodies. Statistically significant differences between untreated or anti-IL-17 treated mice compared to naïve mice are indicated by solid bars; differences between untreated and anti-IL17 treated mice are indicated by dashed bars. p-values associated with bars are indicated.

### DETAILED DESCRIPTION

### I. General Techniques

The techniques and procedures described or referenced herein are generally well understood and commonly employed using conventional methodology by those skilled in the art, such as, for example, the widely utilized methodologies described in Sambrook et al., Molecular Cloning: A Laboratory Manual 3d edition (2001) Cold Spring Harbor Laboratory Press, Cold Spring Harbor, N.Y.; Current Protocols in Molecular Biology (F. M. Ausubel, et al. eds., (2003)); the series Methods in Enzymology (Academic Press, Inc.): PCR 2: A Practical Approach (M. J. MacPherson, B. D. Hames and G. R. Taylor eds. (1995)), Harlow and Lane, eds. (1988) Antibodies, A Laboratory Manual, and Animal Cell Culture (R. I. Freshney, ed. (1987)); Oligonucleotide Synthesis (M. J. Gait, ed., 1984); Methods in Molecular Biology, Humana Press; Cell Biology: A Laboratory Notebook (J. E. Cellis, ed., 1998) Academic Press; Animal Cell Culture (R. I. Freshney), ed., 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds., 1993-8) J. Wiley and Sons; Handbook of Experimental Immunology (D. M. Weir and C. C. Blackwell, eds.); Gene Transfer Vectors for Mammalian Cells (J. M. Miller and M. P. Calos, eds., 1987); PCR: The Polymerase Chain Reaction, (Mullis et al., eds., 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: A Practical Approach (D. Catty, ed., IRL Press, 1988-1989); Monoclonal Antibodies: A Practical Approach (P. Shepherd and C. Dean, eds., Oxford University Press, 2000); Using Antibodies: A Laboratory Manual (E. Harlow and D. Lane (Cold Spring Harbor Laboratory Press, 1999); The Antibodies (M. Zanetti and J. D. Capra, eds., Harwood Academic Publishers, 1995); and Cancer: Principles and Practice of Oncology (V. T. DeVita et al., eds., J. B. Lippincott Company, 1993).

### II. Definitions

Before describing the invention in detail, it is to be understood that this invention is not limited to particular compositions or biological systems, which can, of course, vary. It is also to be understood that the terminology used herein is for the purpose of describing particular embodiments only, and is not intended to be limiting.

As used herein and in the appended claims, the singular forms “a,” “or,” and “the” include plural referents unless the context clearly dictates otherwise.

Reference to “about” a value or parameter herein includes (and describes) variations that are directed to that value or parameter per se. For example, description referring to “about X” includes description of “X”.

It is understood that aspects and variations of the invention described herein include “consisting” and/or “consisting essentially of” aspects and variations.

The term “antagonist” is used in the broadest sense, and includes any molecule that partially or fully blocks, inhibits, or neutralizes a biological activity of a native polypeptide disclosed herein. In a similar manner, the term “agonist” is used in the broadest sense and includes any molecule that mimics a biological activity of a native polypeptide disclosed herein. Suitable agonist or antagonist molecules specifically include agonist or antagonist antibodies or antibody fragments, fragments or amino acid sequence variants of native polypeptides, peptides, antisense oligonucleotides, small organic molecules, etc. Methods for identifying agonists or antagonists of a polypeptide may comprise contacting a polypeptide with a candidate agonist or antagonist molecule and measuring a detectable change in one or more biological activities normally associated with the polypeptide.

The term “aptamer” refers to a nucleic acid molecule that is capable of binding to a target molecule, such as a polypeptide. For example, an aptamer of the invention can specifically bind to an IL-17 or IL-17 receptor polypeptide. The generation and therapeutic use of aptamers are well established in the art. See, e.g., U.S. Pat. No. 5,475,096, and the therapeutic efficacy of Macugen® (Eyetech, New York) for treating age-related macular degeneration.

The term “PD-1 axis binding antagonist” as used herein refers to a molecule that inhibits the interaction of a PD-1 axis binding partner with either one or more of its binding partner, so as to remove T-cell dysfunction resulting from signaling on the PD-1 signaling axis—with a result being to restore or enhance T-cell function (e.g., proliferation, cytokine production, target cell killing). As used herein, a PD-1 axis binding antagonist includes a PD-1 binding antagonist, a PDL1 binding antagonist and a PDL2 binding antagonist.

The term “PD-1 binding antagonist” as used herein refers to a molecule that decreases, blocks, inhibits, abrogates or interferes with signal transduction resulting from the interaction of PD-1 with one or more of its binding partners, such as PDL1, PDL2. In some embodiments, the PD-1 binding antagonist is a molecule that inhibits the binding of PD-1 to its binding partners. In a specific aspect, the PD-1 binding antagonist inhibits the binding of PD-1 to PDL1 and/or PDL2. For example, PD-1 binding antagonists include anti-PD-1 antibodies, antigen binding fragments thereof, immunoadhesins, fusion proteins, oligopeptides and other molecules that decrease, block, inhibit, abrogate or interfere with signal transduction resulting from the interaction of PD-1 with PDL1 and/or PDL2. In one embodiment, a PD-1 binding antagonist reduces the negative co-stimulatory signal mediated by or through cell surface proteins expressed on T lymphocytes mediated signaling through PD-1 so as render a dysfunctional T-cell less dysfunctional (e.g., enhancing effector responses to antigen recognition). In some embodiments, the PD-1 binding antagonist is an anti-PD-1 antibody. In a specific aspect, a PD-1 binding antagonist is nivolumab described herein (also known as MDX-1106-04, MDX-1106, ONO-4538, BMS-936558, and OPDIVO®). In another specific aspect, a PD-1 binding antagonist is pembrolizumab described herein (also known as MK-3475, Merck 3475, KEYTRUDA®, and SCH-900475). In another specific aspect, a PD-1 binding antagonist is CT-011 described herein (also known hBAT or hBAT-1). In yet another specific aspect, a PD-1 binding antagonist is AMP-224 (also known as B7-DCIg) described herein.

The term “PDL1 binding antagonist” as used herein refers to a molecule that decreases, blocks, inhibits, abrogates or interferes with signal transduction resulting from the interaction of PDL1 with either one or more of its binding partners, such as PD-1, B7-1. In some embodiments, a PDL1 binding antagonist is a molecule that inhibits the binding of PDL1 to its binding partners. In a specific aspect, the PDL1 binding antagonist inhibits binding of PDL1 to PD-1 and/or B7-1. In some embodiments, the PDL1 binding antagonists include anti-PDL1 antibodies, antigen binding fragments thereof, immunoadhesins, fusion proteins, oligopeptides and other molecules that decrease, block, inhibit, abrogate or interfere with signal transduction resulting from the interaction of PDL1 with one or more of its binding partners, such as PD-1, B7-1. In one embodiment, a PDL1 binding antagonist reduces the negative co-stimulatory signal mediated by or through cell surface proteins expressed on T lymphocytes mediated signaling through PDL1 so as to render a dysfunctional T-cell less dysfunctional (e.g., enhancing effector responses to antigen recognition). In some embodiments, a PDL1 binding antagonist is an anti-PDL1 antibody. In a specific aspect, an anti-PDL1 antibody is YW243.55.S70 described herein. In another specific aspect, an anti-PDL1 antibody is MDX-1105 described herein (also known as BMS-936559). In still another specific aspect, an anti-PDL1 antibody is MPDL3280A described herein. In still another specific aspect, an anti-PDL1 antibody is MEDI4736 described herein.

The term “PDL2 binding antagonist” as used herein refers to a molecule that decreases, blocks, inhibits, abrogates or interferes with signal transduction resulting from the interaction of PDL2 with either one or more of its binding partners, such as PD-1. In some embodiments, a PDL2 binding antagonist is a molecule that inhibits the binding of PDL2 to its binding partners. In a specific aspect, the PDL2 binding antagonist inhibits binding of PDL2 to PD-1. In some embodiments, the PDL2 antagonists include anti-PDL2 antibodies, antigen binding fragments thereof, immunoadhesins, fusion proteins, oligopeptides and other molecules that decrease, block, inhibit, abrogate or interfere with signal transduction resulting from the interaction of PDL2 with either one or more of its binding partners, such as PD-1. In one embodiment, a PDL2 binding antagonist reduces the negative co-stimulatory signal mediated by or through cell surface proteins expressed on T lymphocytes mediated signaling through PDL2 so as render a dysfunctional T-cell less dysfunctional (e.g., enhancing effector responses to antigen recognition). In some embodiments, a PDL2 binding antagonist is an immunoadhesin.

The term “dysfunction” in the context of immune dysfunction, refers to a state of reduced immune responsiveness to antigenic stimulation. The term includes the common elements of both exhaustion and/or anergy in which antigen recognition may occur, but the ensuing immune response is ineffective to control infection or tumor growth.

The term “dysfunctional”, as used herein, also includes refractory or unresponsive to antigen recognition, specifically, impaired capacity to translate antigen recognition into down-stream T-cell effector functions, such as proliferation, cytokine production (e.g., IL-2) and/or target cell killing.

The term “anergy” refers to the state of unresponsiveness to antigen stimulation resulting from incomplete or insufficient signals delivered through the T-cell receptor (e.g. increase in intracellular Ca⁺² in the absence of ras-activation). T cell anergy can also result upon stimulation with antigen in the absence of co-stimulation, resulting in the cell becoming refractory to subsequent activation by the antigen even in the context of costimulation. The unresponsive state can often be overriden by the presence of Interleukin-2. Anergic T-cells do not undergo clonal expansion and/or acquire effector functions.

The term “exhaustion” refers to T cell exhaustion as a state of T cell dysfunction that arises from sustained TCR signaling that occurs during many chronic infections and cancer. It is distinguished from anergy in that it arises not through incomplete or deficient signaling, but from sustained signaling. It is defined by poor effector function, sustained expression of inhibitory receptors and a transcriptional state distinct from that of functional effector or memory T cells. Exhaustion prevents optimal control of infection and tumors. Exhaustion can result from both extrinsic negative regulatory pathways (e.g., immunoregulatory cytokines) as well as cell intrinsic negative regulatory (costimulatory) pathways.

“Enhancing T-cell function” means to induce, cause or stimulate a T-cell to have a sustained or amplified biological function, or renew or reactivate exhausted or inactive T-cells. Examples of enhancing T-cell function include: increased secretion of γ-interferon from CD8⁺ T-cells, increased proliferation, increased antigen responsiveness (e.g., viral, pathogen, or tumor clearance) relative to such levels before the intervention. In one embodiment, the level of enhancement is as least 50%, alternatively 60%, 70%, 80%, 90%, 100%, 120%, 150%, 200%. The manner of measuring this enhancement is known to one of ordinary skill in the art.

A “T cell dysfunctional disorder” is a disorder or condition of T-cells characterized by decreased responsiveness to antigenic stimulation (e.g., against a tumor expressing an immunogen). In some embodiments, a T-cell dysfunctional disorder is one in which T-cells are anergic or have decreased ability to secrete cytokines, proliferate, or execute cytolytic activity. In a specific aspect, the decreased responsiveness results in ineffective control of a tumor expressing an immunogen. Examples of T cell dysfunctional disorders characterized by T-cell dysfunction include tumor immunity and cancer.

“Tumor immunity” refers to the process in which tumors evade immune recognition and clearance. Thus, as a therapeutic concept, tumor immunity is “treated” when such evasion is attenuated, and the tumors are recognized and attacked by the immune system. Examples of tumor recognition include tumor binding, tumor shrinkage and tumor clearance.

“Immunogenicity” refers to the ability of a particular substance to provoke an immune response. Tumors are immunogenic and enhancing tumor immunogenicity aids in the clearance of the tumor cells by the immune response. Examples of enhancing tumor immunogenicity include but not limited to treatment with a PD-1 axis binding antagonist and an IL-17 binding antagonist.

“Sustained response” refers to the sustained effect on reducing tumor growth after cessation of a treatment. For example, the tumor size may remain to be the same or smaller as compared to the size at the beginning of the administration phase. In some embodiments, the sustained response has a duration at least the same as the treatment duration, at least 1.5×, 2.0×, 2.5×, or 3.0× length of the treatment duration.

As used herein, “cancer” and “cancerous” refer to or describe the physiological condition in mammals that is typically characterized by unregulated cell growth. Included in this definition are benign and malignant cancers as well as dormant tumors or micrometastases. Examples of cancer include but are not limited to, carcinoma, lymphoma, blastoma, sarcoma, and leukemia. More particular examples of such cancers include but are not limited to squamous cell cancer, lung cancer (including small-cell lung cancer, non-small cell lung cancer, adenocarcinoma of the lung, and squamous carcinoma of the lung), melanoma, renal cell carcinoma, cancer of the peritoneum, hepatocellular cancer, gastric or stomach cancer (including gastrointestinal cancer), pancreatic cancer, glioblastoma, cervical cancer, ovarian cancer, liver cancer, bladder cancer, hepatoma, breast cancer, colon cancer, colorectal cancer, endometrial or uterine carcinoma, salivary gland carcinoma, kidney or renal cancer, liver cancer, prostate cancer, vulval cancer, thyroid cancer, hepatic carcinoma and various types of head and neck cancer, as well as B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia); chronic lymphocytic leukemia (CLL); acute lymphoblastic leukemia (ALL); Hairy cell leukemia; chronic myeloblastic leukemia; and post-transplant lymphoproliferative disorder (PTLD), as well as abnormal vascular proliferation associated with phakomatoses, edema (such as that associated with brain tumors), and Meigs' syndrome. Examples of cancer may include primary tumors of any of the above types of cancer or metastatic tumors at a second site derived from any of the above types of cancer.

As used herein, “metastasis” is meant the spread of cancer from its primary site to other places in the body. Cancer cells can break away from a primary tumor, penetrate into lymphatic and blood vessels, circulate through the bloodstream, and grow in a distant focus (metastasize) in normal tissues elsewhere in the body. Metastasis can be local or distant. Metastasis is a sequential process, contingent on tumor cells breaking off from the primary tumor, traveling through the bloodstream, and stopping at a distant site. At the new site, the cells establish a blood supply and can grow to form a life-threatening mass. Both stimulatory and inhibitory molecular pathways within the tumor cell regulate this behavior, and interactions between the tumor cell and host cells in the distant site are also significant.

The term “antibody” includes monoclonal antibodies (including full length antibodies which have an immunoglobulin Fc region), antibody compositions with polyepitopic specificity, multispecific antibodies (e.g., bispecific antibodies, diabodies, and single-chain molecules, as well as antibody fragments (e.g., Fab, F(ab′)₂, and Fv). The term “immunoglobulin” (Ig) is used interchangeably with “antibody” herein.

The basic 4-chain antibody unit is a heterotetrameric glycoprotein composed of two identical light (L) chains and two identical heavy (H) chains. An IgM antibody consists of 5 of the basic heterotetramer units along with an additional polypeptide called a J chain, and contains 10 antigen binding sites, while IgA antibodies comprise from 2-5 of the basic 4-chain units which can polymerize to form polyvalent assemblages in combination with the J chain. In the case of IgGs, the 4-chain unit is generally about 150,000 daltons. Each L chain is linked to an H chain by one covalent disulfide bond, while the two H chains are linked to each other by one or more disulfide bonds depending on the H chain isotype. Each H and L chain also has regularly spaced intrachain disulfide bridges. Each H chain has at the N-terminus, a variable domain (V_{H}) followed by three constant domains (C_{H}) for each of the α and γ chains and four C_{H} domains for μ and ε isotypes. Each L chain has at the N-terminus, a variable domain (V_{L}) followed by a constant domain at its other end. The V_{L} is aligned with the V_{H} and the C_{L} is aligned with the first constant domain of the heavy chain (C_{H}1). Particular amino acid residues are believed to form an interface between the light chain and heavy chain variable domains. The pairing of a V_{H} and V_{L} together forms a single antigen-binding site. For the structure and properties of the different classes of antibodies, see e.g., Basic and Clinical Immunology, 8th Edition, Daniel P. Sties, Abba I. Terr and Tristram G. Parsolw (eds), Appleton & Lange, Norwalk, Conn., 1994, page 71 and Chapter 6. The L chain from any vertebrate species can be assigned to one of two clearly distinct types, called kappa and lambda, based on the amino acid sequences of their constant domains. Depending on the amino acid sequence of the constant domain of their heavy chains (CH), immunoglobulins can be assigned to different classes or isotypes. There are five classes of immunoglobulins: IgA, IgD, IgE, IgG and IgM, having heavy chains designated α, δ, ε, γ and μ, respectively. The γ and α classes are further divided into subclasses on the basis of relatively minor differences in the CH sequence and function, e.g., humans express the following subclasses: IgG1, IgG2A, IgG2B, IgG3, IgG4, IgA1 and IgA2.

The “variable region” or “variable domain” of an antibody refers to the amino-terminal domains of the heavy or light chain of the antibody. The variable domains of the heavy chain and light chain may be referred to as “VH” and “VL”, respectively. These domains are generally the most variable parts of the antibody (relative to other antibodies of the same class) and contain the antigen binding sites.

The term “variable” refers to the fact that certain segments of the variable domains differ extensively in sequence among antibodies. The V domain mediates antigen binding and defines the specificity of a particular antibody for its particular antigen. However, the variability is not evenly distributed across the entire span of the variable domains. Instead, it is concentrated in three segments called hypervariable regions (HVRs) both in the light-chain and the heavy chain variable domains. The more highly conserved portions of variable domains are called the framework regions (FR). The variable domains of native heavy and light chains each comprise four FR regions, largely adopting a beta-sheet configuration, connected by three HVRs, which form loops connecting, and in some cases forming part of, the beta-sheet structure. The HVRs in each chain are held together in close proximity by the FR regions and, with the HVRs from the other chain, contribute to the formation of the antigen binding site of antibodies (see Kabat et al., Sequences of Immunological Interest, Fifth Edition, National Institute of Health, Bethesda, Md. (1991)). The constant domains are not involved directly in the binding of antibody to an antigen, but exhibit various effector functions, such as participation of the antibody in antibody-dependent cellular toxicity.

The term “monoclonal antibody” as used herein refers to an antibody obtained from a population of substantially homogeneous antibodies, i.e., the individual antibodies comprising the population are identical except for possible naturally occurring mutations and/or post-translation modifications (e.g., isomerizations, amidations) that may be present in minor amounts. Monoclonal antibodies are highly specific, being directed against a single antigenic site. In contrast to polyclonal antibody preparations which typically include different antibodies directed against different determinants (epitopes), each monoclonal antibody is directed against a single determinant on the antigen. In addition to their specificity, the monoclonal antibodies are advantageous in that they are synthesized by the hybridoma culture, uncontaminated by other immunoglobulins. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by a variety of techniques, including, for example, the hybridoma method (e.g., Kohler and Milstein, Nature, 256:495-97 (1975); Hongo et al., Hybridoma, 14 (3): 253-260 (1995), Harlow et al., Antibodies: A Laboratory Manual, (Cold Spring Harbor Laboratory Press, 2^{nd} ed. 1988); Hammerling et al., in: Monoclonal Antibodies and T-Cell Hybridomas 563-681 (Elsevier, N.Y., 1981)), recombinant DNA methods (see, e.g., U.S. Pat. No. 4,816,567), phage-display technologies (see, e.g., Clackson et al., Nature, 352: 624-628 (1991); Marks et al., J. Mol. Biol. 222: 581-597 (1992); Sidhu et al., J. Mol. Biol. 338(2): 299-310 (2004); Lee et al., J. Mol. Biol. 340(5): 1073-1093 (2004); Fellouse, Proc. Natl. Acad. Sci. USA 101(34): 12467-12472 (2004); and Lee et al., J. Immunol. Methods 284(1-2): 119-132 (2004), and technologies for producing human or human-like antibodies in animals that have parts or all of the human immunoglobulin loci or genes encoding human immunoglobulin sequences (see, e.g., WO 1998/24893; WO 1996/34096; WO 1996/33735; WO 1991/10741; Jakobovits et al., Proc. Natl. Acad. Sci. USA 90: 2551 (1993); Jakobovits et al., Nature 362: 255-258 (1993); Bruggemann et al., Year in Immunol. 7:33 (1993); U.S. Pat. Nos. 5,545,807; 5,545,806; 5,569,825; 5,625,126; 5,633,425; and U.S. Pat. No. 5,661,016; Marks et al., Bio/Technology 10: 779-783 (1992); Lonberg et al., Nature 368: 856-859 (1994); Morrison, Nature 368: 812-813 (1994); Fishwild et al., Nature Biotechnol. 14: 845-851 (1996); Neuberger, Nature Biotechnol. 14: 826 (1996); and Lonberg and Huszar, Intern. Rev. Immunol. 13: 65-93 (1995).

The term “naked antibody” refers to an antibody that is not conjugated to a cytotoxic moiety or radiolabel.

The terms “full-length antibody,” “intact antibody” or “whole antibody” are used interchangeably to refer to an antibody in its substantially intact form, as opposed to an antibody fragment. Specifically whole antibodies include those with heavy and light chains including an Fc region. The constant domains may be native sequence constant domains (e.g., human native sequence constant domains) or amino acid sequence variants thereof. In some cases, the intact antibody may have one or more effector functions.

An “antibody fragment” comprises a portion of an intact antibody, preferably the antigen binding and/or the variable region of the intact antibody. Examples of antibody fragments include Fab, Fab′, F(ab′)₂ and Fv fragments; diabodies; linear antibodies (see U.S. Pat. No. 5,641,870, Example 2; Zapata et al., Protein Eng. 8(10): 1057-1062 [1995]); single-chain antibody molecules and multispecific antibodies formed from antibody fragments. Papain digestion of antibodies produced two identical antigen-binding fragments, called “Fab” fragments, and a residual “Fc” fragment, a designation reflecting the ability to crystallize readily. The Fab fragment consists of an entire L chain along with the variable region domain of the H chain (V_{H}), and the first constant domain of one heavy chain (C_{H}1). Each Fab fragment is monovalent with respect to antigen binding, i.e., it has a single antigen-binding site. Pepsin treatment of an antibody yields a single large F(ab′)₂ fragment which roughly corresponds to two disulfide linked Fab fragments having different antigen-binding activity and is still capable of cross-linking antigen. Fab′ fragments differ from Fab fragments by having a few additional residues at the carboxy terminus of the C_{H}1 domain including one or more cysteines from the antibody hinge region. Fab′-SH is the designation herein for Fab′ in which the cysteine residue(s) of the constant domains bear a free thiol group. F(ab′)₂ antibody fragments originally were produced as pairs of Fab′ fragments which have hinge cysteines between them. Other chemical couplings of antibody fragments are also known.

The Fc fragment comprises the carboxy-terminal portions of both H chains held together by disulfides. The effector functions of antibodies are determined by sequences in the Fc region, the region which is also recognized by Fc receptors (FcR) found on certain types of cells.

“Fv” is the minimum antibody fragment which contains a complete antigen-recognition and -binding site. This fragment consists of a dimer of one heavy- and one light-chain variable region domain in tight, non-covalent association. From the folding of these two domains emanate six hypervariable loops (3 loops each from the H and L chain) that contribute the amino acid residues for antigen binding and confer antigen binding specificity to the antibody. However, even a single variable domain (or half of an Fv comprising only three HVRs specific for an antigen) has the ability to recognize and bind antigen, although at a lower affinity than the entire binding site.

“Single-chain Fv” also abbreviated as “sFv” or “scFv” are antibody fragments that comprise the V_{H} and V_{L} antibody domains connected into a single polypeptide chain. Preferably, the sFv polypeptide further comprises a polypeptide linker between the V_{H} and V_{L} domains which enables the sFv to form the desired structure for antigen binding. For a review of the sFv, see Pluckthun in The Pharmacology of Monoclonal Antibodies, vol. 113, Rosenburg and Moore eds., Springer-Verlag, New York, pp. 269-315 (1994).

“Functional fragments” of the antibodies of the invention comprise a portion of an intact antibody, generally including the antigen binding or variable region of the intact antibody or the Fc region of an antibody which retains or has modified FcR binding capability. Examples of antibody fragments include linear antibody, single-chain antibody molecules and multispecific antibodies formed from antibody fragments.

The term “diabodies” refers to small antibody fragments prepared by constructing sFv fragments (see preceding paragraph) with short linkers (about 5-10) residues) between the V_{H} and V_{L} domains such that inter-chain but not intra-chain pairing of the V domains is achieved, thereby resulting in a bivalent fragment, i.e., a fragment having two antigen-binding sites. Bispecific diabodies are heterodimers of two “crossover” sFv fragments in which the V_{H} and V_{L} domains of the two antibodies are present on different polypeptide chains. Diabodies are described in greater detail in, for example, EP 404,097; WO 93/11161; Hollinger et al., Proc. Natl. Acad. Sci. USA 90: 6444-6448 (1993).

The monoclonal antibodies herein specifically include “chimeric” antibodies (immunoglobulins) in which a portion of the heavy and/or light chain is identical with or homologous to corresponding sequences in antibodies derived from a particular species or belonging to a particular antibody class or subclass, while the remainder of the chain(s) is(are) identical with or homologous to corresponding sequences in antibodies derived from another species or belonging to another antibody class or subclass, as well as fragments of such antibodies, so long as they exhibit the desired biological activity (U.S. Pat. No. 4,816,567; Morrison et al., Proc. Natl. Acad. Sci. USA, 81:6851-6855 (1984)). Chimeric antibodies of interest herein include PRIMATIZED® antibodies wherein the antigen-binding region of the antibody is derived from an antibody produced by, e.g., immunizing macaque monkeys with an antigen of interest. As used herein, “humanized antibody” is used a subset of “chimeric antibodies.”

“Humanized” forms of non-human (e.g., murine) antibodies are chimeric antibodies that contain minimal sequence derived from non-human immunoglobulin. In one embodiment, a humanized antibody is a human immunoglobulin (recipient antibody) in which residues from an HVR (hereinafter defined) of the recipient are replaced by residues from an HVR of a non-human species (donor antibody) such as mouse, rat, rabbit or non-human primate having the desired specificity, affinity, and/or capacity. In some instances, framework (“FR”) residues of the human immunoglobulin are replaced by corresponding non-human residues. Furthermore, humanized antibodies may comprise residues that are not found in the recipient antibody or in the donor antibody. These modifications may be made to further refine antibody performance, such as binding affinity. In general, a humanized antibody will comprise substantially all of at least one, and typically two, variable domains, in which all or substantially all of the hypervariable loops correspond to those of a non-human immunoglobulin sequence, and all or substantially all of the FR regions are those of a human immunoglobulin sequence, although the FR regions may include one or more individual FR residue substitutions that improve antibody performance, such as binding affinity, isomerization, immunogenicity, etc. The number of these amino acid substitutions in the FR are typically no more than 6 in the H chain, and in the L chain, no more than 3. The humanized antibody optionally will also comprise at least a portion of an immunoglobulin constant region (Fc), typically that of a human immunoglobulin. For further details, see, e.g., Jones et al., Nature 321:522-525 (1986); Riechmann et al., Nature 332:323-329 (1988); and Presta, Curr. Op. Struct. Biol. 2:593-596 (1992). See also, for example, Vaswani and Hamilton, Ann. Allergy, Asthma & Immunol. 1:105-115 (1998); Harris, Biochem. Soc. Transactions 23:1035-1038 (1995); Hurle and Gross, Curr. Op. Biotech. 5:428-433 (1994); and U.S. Pat. Nos. 6,982,321 and 7,087,409.

A “human antibody” is an antibody that possesses an amino-acid sequence corresponding to that of an antibody produced by a human and/or has been made using any of the techniques for making human antibodies as disclosed herein. This definition of a human antibody specifically excludes a humanized antibody comprising non-human antigen-binding residues. Human antibodies can be produced using various techniques known in the art, including phage-display libraries. Hoogenboom and Winter, J. Mol. Biol., 227:381 (1991); Marks et al., J. Mol. Biol., 222:581 (1991). Also available for the preparation of human monoclonal antibodies are methods described in Cole et al., Monoclonal Antibodies and Cancer Therapy, Alan R. Liss, p. 77 (1985); Boerner et al., J. Immunol., 147(1):86-95 (1991). See also van Dijk and van de Winkel, Curr. Opin. Pharmacol., 5: 368-74 (2001). Human antibodies can be prepared by administering the antigen to a transgenic animal that has been modified to produce such antibodies in response to antigenic challenge, but whose endogenous loci have been disabled, e.g., immunized xenomice (see, e.g., U.S. Pat. Nos. 6,075,181 and 6,150,584 regarding XENOMOUSE™ technology). See also, for example, Li et al., Proc. Natl. Acad. Sci. USA, 103:3557-3562 (2006) regarding human antibodies generated via a human B-cell hybridoma technology.

The term “hypervariable region,” “HVR,” or “HV,” when used herein refers to the regions of an antibody variable domain which are hypervariable in sequence and/or form structurally defined loops. Generally, antibodies comprise six HVRs; three in the VH (H1, H2, H3), and three in the VL (L1, L2, L3). In native antibodies, H3 and L3 display the most diversity of the six HVRs, and H3 in particular is believed to play a unique role in conferring fine specificity to antibodies. See, e.g., Xu et al., Immunity 13:37-45 (2000); Johnson and Wu, in Methods in Molecular Biology 248:1-25 (Lo, ed., Human Press, Totowa, N.J., 2003). Indeed, naturally occurring camelid antibodies consisting of a heavy chain only are functional and stable in the absence of light chain. See, e.g., Hamers-Casterman et al., Nature 363:446-448 (1993); Sheriff et al., Nature Struct. Biol. 3:733-736 (1996).

A number of HVR delineations are in use and are encompassed herein. The Kabat Complementarity Determining Regions (CDRs) are based on sequence variability and are the most commonly used (Kabat et al., Sequences of Proteins of Immunological Interest, 5th Ed. Public Health Service, National Institutes of Health, Bethesda, Md. (1991)). Chothia refers instead to the location of the structural loops (Chothia and Lesk, J. Mol. Biol. 196:901-917 (1987)). The AbM HVRs represent a compromise between the Kabat HVRs and Chothia structural loops, and are used by Oxford Molecular's AbM antibody modeling software. The “contact” HVRs are based on an analysis of the available complex crystal structures. The residues from each of these HVRs are noted below.

| Loop | Kabat | AbM | Chothia | Contact |
|---|---|---|---|---|
| L1 | L24-L34 | L24-L34 | L26-L32 | L30-L36 |
| L2 | L50-L56 | L50-L56 | L50-L52 | L46-L55 |
| L3 | L89-L97 | L89-L97 | L91-L96 | L89-L96 |
| H1 | H31-H35B | H26-H35B | H26-H32 | H30-H35B (Kabat |
| | | | | numbering) |
| H1 | H31-H35 | H26-H35 | H26-H32 | H30-H35 (Chothia |
| | | | | numbering) |
| H2 | H50-H65 | H50-H58 | H53-H55 | H47-H58 |
| H3 | H95-H102 | H95-H102 | H96-H101 | H93-H101 |

HVRs may comprise “extended HVRs” as follows: 24-36 or 24-34 (L1), 46-56 or 50-56 (L2) and 89-97 or 89-96 (L3) in the VL and 26-35 (H1), 50-65 or 49-65 (H2) and 93-102, 94-102, or 95-102 (H3) in the VH. The variable domain residues are numbered according to Kabat et al., supra, for each of these definitions.

The expression “variable-domain residue-numbering as in Kabat” or “amino-acid-position numbering as in Kabat,” and variations thereof, refers to the numbering system used for heavy-chain variable domains or light-chain variable domains of the compilation of antibodies in Kabat et al., supra. Using this numbering system, the actual linear amino acid sequence may contain fewer or additional amino acids corresponding to a shortening of, or insertion into, a FR or HVR of the variable domain. For example, a heavy-chain variable domain may include a single amino acid insert (residue 52a according to Kabat) after residue 52 of H2 and inserted residues (e.g. residues 82a, 82b, and 82c, etc. according to Kabat) after heavy-chain FR residue 82. The Kabat numbering of residues may be determined for a given antibody by alignment at regions of homology of the sequence of the antibody with a “standard” Kabat numbered sequence.

“Framework” or “FR” residues are those variable-domain residues other than the HVR residues as herein defined.

A “human consensus framework” or “acceptor human framework” is a framework that represents the most commonly occurring amino acid residues in a selection of human immunoglobulin VL or VH framework sequences. Generally, the selection of human immunoglobulin VL or VH sequences is from a subgroup of variable domain sequences. Generally, the subgroup of sequences is a subgroup as in Kabat et al., Sequences of Proteins of Immunological Interest, 5^{th} Ed. Public Health Service, National Institutes of Health, Bethesda, Md. (1991). Examples include for the VL, the subgroup may be subgroup kappa I, kappa II, kappa III or kappa IV as in Kabat et al., supra. Additionally, for the VH, the subgroup may be subgroup I, subgroup II, or subgroup III as in Kabat et al., supra. Alternatively, a human consensus framework can be derived from the above in which particular residues, such as when a human framework residue is selected based on its homology to the donor framework by aligning the donor framework sequence with a collection of various human framework sequences. An acceptor human framework “derived from” a human immunoglobulin framework or a human consensus framework may comprise the same amino acid sequence thereof, or it may contain pre-existing amino acid sequence changes. In some embodiments, the number of pre-existing amino acid changes are 10 or less, 9 or less, 8 or less, 7 or less, 6 or less, 5 or less, 4 or less, 3 or less, or 2 or less.

A “VH subgroup III consensus framework” comprises the consensus sequence obtained from the amino acid sequences in variable heavy subgroup III of Kabat et al., supra. In one embodiment, the VH subgroup III consensus framework amino acid sequence comprises at least a portion or all of each of the following sequences:

(SEQ ID NO: 4)
EVQLVESGGGLVQPGGSLRLSCAAS (HC-FR1),
(SEQ ID NO: 5),
WVRQAPGKGLEWV (HC-FR2),
(HC-FR3, SEQ ID NO: 6),
RFTISADTSKNTAYLQMNSLRAEDTAVYYCAR
(SEQ ID NO: 7).
WGQGTLVTVSA (HC-FR4),

A “VL kappa I consensus framework” comprises the consensus sequence obtained from the amino acid sequences in variable light kappa subgroup I of Kabat et al., supra. In one embodiment, the VH subgroup I consensus framework amino acid sequence comprises at least a portion or all of each of the following sequences: DIQMTQSPSSLSASVGDRVTITC (LC-FR1) (SEQ ID NO:11), WYQQKPGKAPKLLIY (LC-FR2) (SEQ ID NO:12), GVPSRFSGSGSGTDFTLTISSLQPEDFATYYC (LC-FR3)(SEQ ID NO:13), FGQGTKVEIKR (LC-FR4)(SEQ ID NO:14).

An “amino-acid modification” at a specified position, e.g. of the Fc region, refers to the substitution or deletion of the specified residue, or the insertion of at least one amino acid residue adjacent the specified residue. Insertion “adjacent” to a specified residue means insertion within one to two residues thereof. The insertion may be N-terminal or C-terminal to the specified residue. The preferred amino acid modification herein is a substitution.

An “affinity-matured” antibody is one with one or more alterations in one or more HVRs thereof that result in an improvement in the affinity of the antibody for antigen, compared to a parent antibody that does not possess those alteration(s). In one embodiment, an affinity-matured antibody has nanomolar or even picomolar affinities for the target antigen. Affinity-matured antibodies are produced by procedures known in the art. For example, Marks et al., Bio/Technology 10:779-783 (1992) describes affinity maturation by VH- and VL-domain shuffling. Random mutagenesis of HVR and/or framework residues is described by, for example: Barbas et al. Proc Nat. Acad. Sci. USA 91:3809-3813 (1994); Schier et al. Gene 169:147-155 (1995); Yelton et al. J. Immunol. 155:1994-2004 (1995); Jackson et al., J. Immunol. 154(7):3310-9 (1995); and Hawkins et al, J. Mol. Biol. 226:889-896 (1992).

As use herein, the term “specifically binds to” or is “specific for” refers to measurable and reproducible interactions such as binding between a target and an antibody, which is determinative of the presence of the target in the presence of a heterogeneous population of molecules including biological molecules. For example, an antibody that specifically binds to a target (which can be an epitope) is an antibody that binds this target with greater affinity, avidity, more readily, and/or with greater duration than it binds to other targets. In one embodiment, the extent of binding of an antibody to an unrelated target is less than about 10% of the binding of the antibody to the target as measured, e.g., by a radioimmunoassay (RIA). In certain embodiments, an antibody that specifically binds to a target has a dissociation constant (Kd) of ≦1 μM≦100 nM, ≦10 nM, ≦1 nM, or ≦0.1 nM. In certain embodiments, an antibody specifically binds to an epitope on a protein that is conserved among the protein from different species. In another embodiment, specific binding can include, but does not require exclusive binding.

As used herein, the term “immunoadhesin” designates antibody-like molecules which combine the binding specificity of a heterologous protein (an “adhesin”) with the effector functions of immunoglobulin constant domains. Structurally, the immunoadhesins comprise a fusion of an amino acid sequence with the desired binding specificity which is other than the antigen recognition and binding site of an antibody (i.e., is “heterologous”), and an immunoglobulin constant domain sequence. The adhesin part of an immunoadhesin molecule typically is a contiguous amino acid sequence comprising at least the binding site of a receptor or a ligand. The immunoglobulin constant domain sequence in the immunoadhesin may be obtained from any immunoglobulin, such as IgG-1, IgG-2 (including IgG2A and IgG2B), IgG-3, or IgG-4 subtypes, IgA (including IgA-1 and IgA-2), IgE, IgD or IgM. The Ig fusions preferably include the substitution of a domain of a polypeptide or antibody described herein in the place of at least one variable region within an Ig molecule. In a particularly preferred embodiment, the immunoglobulin fusion includes the hinge, CH2 and CH3, or the hinge, CH1, CH2 and CH3 regions of an IgG1 molecule. For the production of immunoglobulin fusions see also U.S. Pat. No. 5,428,130 issued Jun. 27, 1995 Immunoadhesin combinations of Ig Fc and ECD of cell surface receptors are sometimes termed soluble receptors.

A “fusion protein” and a “fusion polypeptide” refer to a polypeptide having two portions covalently linked together, where each of the portions is a polypeptide having a different property. The property may be a biological property, such as activity in vitro or in vivo. The property may also be simple chemical or physical property, such as binding to a target molecule, catalysis of a reaction, etc. The two portions may be linked directly by a single peptide bond or through a peptide linker but are in reading frame with each other.

A “PD-1 oligopeptide,” “PDL1 oligopeptide,” or “PDL2 oligopeptide” is an oligopeptide that binds, preferably specifically, to a PD-1, PDL1 or PDL2 negative costimulatory polypeptide, respectively, including a receptor, ligand or signaling component, respectively, as described herein. Such oligopeptides may be chemically synthesized using known oligopeptide synthesis methodology or may be prepared and purified using recombinant technology. Such oligopeptides are usually at least about 5 amino acids in length, alternatively at least about 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 amino acids in length or more. Such oligopeptides may be identified using well known techniques. In this regard, it is noted that techniques for screening oligopeptide libraries for oligopeptides that are capable of specifically binding to a polypeptide target are well known in the art (see, e.g., U.S. Pat. Nos. 5,556,762, 5,750,373, 4,708,871, 4,833,092, 5,223,409, 5,403,484, 5,571,689, 5,663,143; PCT Publication Nos. WO 84/03506 and WO84/03564; Geysen et al., Proc. Natl. Acad. Sci. U.S.A., 81:3998-4002 (1984); Geysen et al., Proc. Natl. Acad. Sci. U.S.A., 82:178-182 (1985); Geysen et al., in Synthetic Peptides as Antigens, 130-149 (1986); Geysen et al., J. Immunol. Meth., 102:259-274 (1987); Schoofs et al., J. Immunol., 140:611-616 (1988), Cwirla, S. E. et al. Proc. Natl. Acad. Sci. USA, 87:6378 (1990); Lowman, H. B. et al. Biochemistry, 30:10832 (1991); Clackson, T. et al. Nature, 352: 624 (1991); Marks, J. D. et al., J. Mol. Biol., 222:581 (1991); Kang, A. S. et al. Proc. Natl. Acad. Sci. USA, 88:8363 (1991), and Smith, G. P., Current Opin. Biotechnol., 2:668 (1991).

A “blocking” antibody or an “antagonist” antibody is one that inhibits or reduces a biological activity of the antigen it binds. In some embodiments, blocking antibodies or antagonist antibodies substantially or completely inhibit the biological activity of the antigen. The anti-PDL1 antibodies of the invention block the signaling through PD-1 so as to restore a functional response by T-cells (e.g., proliferation, cytokine production, target cell killing) from a dysfunctional state to antigen stimulation.

An “agonist” or activating antibody is one that enhances or initiates signaling by the antigen to which it binds. In some embodiments, agonist antibodies cause or activate signaling without the presence of the natural ligand.

The term “Fc region” herein is used to define a C-terminal region of an immunoglobulin heavy chain, including native-sequence Fc regions and variant Fc regions. Although the boundaries of the Fc region of an immunoglobulin heavy chain might vary, the human IgG heavy-chain Fc region is usually defined to stretch from an amino acid residue at position Cys226, or from Pro230, to the carboxyl-terminus thereof. The C-terminal lysine (residue 447 according to the EU numbering system) of the Fc region may be removed, for example, during production or purification of the antibody, or by recombinantly engineering the nucleic acid encoding a heavy chain of the antibody. Accordingly, a composition of intact antibodies may comprise antibody populations with all K447 residues removed, antibody populations with no K447 residues removed, and antibody populations having a mixture of antibodies with and without the K447 residue. Suitable native-sequence Fc regions for use in the antibodies of the invention include human IgG1, IgG2 (IgG2A, IgG2B), IgG3 and IgG4.

“Fc receptor” or “FcR” describes a receptor that binds to the Fc region of an antibody. The preferred FcR is a native sequence human FcR. Moreover, a preferred FcR is one which binds an IgG antibody (a gamma receptor) and includes receptors of the FcγRI, FcγRII, and FcγRIII subclasses, including allelic variants and alternatively spliced forms of these receptors, FcγRII receptors include FcγRIIA (an “activating receptor”) and FcγRIIB (an “inhibiting receptor”), which have similar amino acid sequences that differ primarily in the cytoplasmic domains thereof. Activating receptor FcγRIIA contains an immunoreceptor tyrosine-based activation motif (ITAM) in its cytoplasmic domain. Inhibiting receptor FcγRIIB contains an immunoreceptor tyrosine-based inhibition motif (ITIM) in its cytoplasmic domain. (see M. Daëron, Annu. Rev. Immunol. 15:203-234 (1997). FcRs are reviewed in Ravetch and Kinet, Annu. Rev. Immunol. 9: 457-92 (1991); Capel et al., Immunomethods 4: 25-34 (1994); and de Haas et al., J. Lab. Clin. Med. 126: 330-41 (1995). Other FcRs, including those to be identified in the future, are encompassed by the term “FcR” herein.

The term “Fc receptor” or “FcR” also includes the neonatal receptor, FcRn, which is responsible for the transfer of maternal IgGs to the fetus. Guyer et al., J. Immunol. 117: 587 (1976) and Kim et al., J. Immunol. 24: 249 (1994). Methods of measuring binding to FcRn are known (see, e.g., Ghetie and Ward, Immunol. Today 18: (12): 592-8 (1997); Ghetie et al., Nature Biotechnology 15 (7): 637-40 (1997); Hinton et al., J. Biol. Chem. 279 (8): 6213-6 (2004); WO 2004/92219 (Hinton et al.). Binding to FcRn in vivo and serum half-life of human FcRn high-affinity binding polypeptides can be assayed, e.g., in transgenic mice or transfected human cell lines expressing human FcRn, or in primates to which the polypeptides having a variant Fc region are administered. WO 2004/42072 (Presta) describes antibody variants which improved or diminished binding to FcRs. See also, e.g., Shields et al., J. Biol. Chem. 9(2): 6591-6604 (2001).

The phrase “substantially reduced,” or “substantially different,” as used herein, denotes a sufficiently high degree of difference between two numeric values (generally one associated with a molecule and the other associated with a reference/comparator molecule) such that one of skill in the art would consider the difference between the two values to be of statistical significance within the context of the biological characteristic measured by said values (e.g., Kd values). The difference between said two values is, for example, greater than about 10%, greater than about 20%, greater than about 30%, greater than about 40%, and/or greater than about 50% as a function of the value for the reference/comparator molecule.

The term “substantially similar” or “substantially the same,” as used herein, denotes a sufficiently high degree of similarity between two numeric values (for example, one associated with an antibody of the invention and the other associated with a reference/comparator antibody), such that one of skill in the art would consider the difference between the two values to be of little or no biological and/or statistical significance within the context of the biological characteristic measured by said values (e.g., Kd values). The difference between said two values is, for example, less than about 50%, less than about 40%, less than about 30%, less than about 20%, and/or less than about 10% as a function of the reference/comparator value.

“Carriers” as used herein include pharmaceutically acceptable carriers, excipients, or stabilizers that are nontoxic to the cell or mammal being exposed thereto at the dosages and concentrations employed. Often the physiologically acceptable carrier is an aqueous pH buffered solution. Examples of physiologically acceptable carriers include buffers such as phosphate, citrate, and other organic acids; antioxidants including ascorbic acid; low molecular weight (less than about 10 residues) polypeptide; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN™, polyethylene glycol (PEG), and PLURONICS™.

A “package insert” refers to instructions customarily included in commercial packages of medicaments that contain information about the indications customarily included in commercial packages of medicaments that contain information about the indications, usage, dosage, administration, contraindications, other medicaments to be combined with the packaged product, and/or warnings concerning the use of such medicaments, etc.

As used herein, the term “treatment” or “treating” refers to clinical intervention designed to alter the natural course of the individual or cell being treated during the course of clinical pathology, e.g., cancer or tumor immunity. Desirable effects of treatment include decreasing the rate of disease progression, ameliorating or palliating the disease state, and remission or improved prognosis. For example, an individual is successfully “treated” if one or more symptoms associated with cancer are mitigated or eliminated, including, but are not limited to, reducing the proliferation of (or destroying) cancerous cells, decreasing symptoms resulting from the disease, increasing the quality of life of those suffering from the disease, decreasing the dose of other medications required to treat the disease, delaying the progression of the disease, and/or prolonging survival of individuals.

As used herein, “delaying progression of a disease” means to defer, hinder, slow, retard, stabilize, and/or postpone development of the disease (such as cancer or tumor immunity). This delay can be of varying lengths of time, depending on the history of the disease and/or individual being treated. As is evident to one skilled in the art, a sufficient or significant delay can, in effect, encompass prevention, in that the individual does not develop the disease. For example, a late stage cancer, such as development of metastasis, may be delayed.

As used herein, “reducing or inhibiting cancer relapse” means to reduce or inhibit tumor or cancer relapse or tumor or cancer progression. As disclosed herein, cancer relapse and/or cancer progression include, without limitation, cancer metastasis.

An “effective amount” is at least the minimum concentration required to effect a measurable improvement or prevention of a particular disorder. An effective amount herein may vary according to factors such as the disease state, age, sex, and weight of the patient, and the ability of the antibody to elicit a desired response in the individual. An effective amount is also one in which any toxic or detrimental effects of the treatment are outweighed by the therapeutically beneficial effects. For prophylactic use, beneficial or desired results include results such as eliminating or reducing the risk, lessening the severity, or delaying the onset of the disease, including biochemical, histological and/or behavioral symptoms of the disease, its complications and intermediate pathological phenotypes presenting during development of the disease. For therapeutic use, beneficial or desired results include clinical results such as decreasing one or more symptoms resulting from the disease, increasing the quality of life of those suffering from the disease, decreasing the dose of other medications required to treat the disease, enhancing effect of another medication such as via targeting, delaying the progression of the disease, and/or prolonging survival. In the case of cancer or tumor, an effective amount of the drug may have the effect in reducing the number of cancer cells; reducing the tumor size; inhibiting (i.e., slow to some extent or desirably stop) cancer cell infiltration into peripheral organs; inhibit (i.e., slow to some extent and desirably stop) tumor metastasis; inhibiting to some extent tumor growth; and/or relieving to some extent one or more of the symptoms associated with the disorder. An effective amount can be administered in one or more administrations. For purposes of this invention, an effective amount of drug, compound, or pharmaceutical composition is an amount sufficient to accomplish prophylactic or therapeutic treatment either directly or indirectly. As is understood in the clinical context, an effective amount of a drug, compound, or pharmaceutical composition may or may not be achieved in conjunction with another drug, compound, or pharmaceutical composition. Thus, an “effective amount” may be considered in the context of administering one or more therapeutic agents, and a single agent may be considered to be given in an effective amount if, in conjunction with one or more other agents, a desirable result may be or is achieved.

As used herein, “in conjunction with” refers to administration of one treatment modality in addition to another treatment modality. As such, “in conjunction with” refers to administration of one treatment modality before, during, or after administration of the other treatment modality to the individual. The term “in combination with” may be used interchangeably herein.

As used herein, the terms “individual” and “subject” may be used interchangeably and refer to a mammal, including, but not limited to, a human or non-human mammal, such as a bovine, equine, canine, ovine, or feline. Preferably, the individual or subject is a human. Patients are also individuals or subjects herein.

As used herein, “complete response” or “CR” refers to disappearance of all target lesions (e.g., human lesions); “partial response” or “PR” refers to at least a 30% decrease in the sum of the longest diameters (SLD) of target lesions (e.g., human lesions), taking as reference the baseline SLD; and “stable disease” or “SD” refers to neither sufficient shrinkage of target lesions to qualify for PR, nor sufficient increase to qualify for PD, taking as reference the smallest SLD since the treatment started (e.g., human lesions).

As used herein, “progressive disease” or “PD” refers to at least a 20% increase in the SLD of target lesions (e.g., human lesions), taking as reference the smallest SLD recorded since the treatment started or the presence of one or more new lesions.

As used herein, “progression free survival” (PFS) refers to the length of time during and after treatment during which the disease being treated (e.g., cancer) does not get worse. Progression-free survival may include the amount of time patients have experienced a complete response or a partial response, as well as the amount of time patients have experienced stable disease.

As used herein, “overall response rate” (ORR) refers to the sum of complete response (CR) rate and partial response (PR) rate.

As used herein, “overall survival” refers to the percentage of individuals in a group who are likely to be alive after a particular duration of time.

As used herein, “RECIST response” refers to a response determined according to the published set of guidelines for determining the status of a tumor in a cancer patient, i.e., responding, stabilizing, or progressing. For a more detailed discussion of these guidelines, see Therasse, P., et al. J. Natl. Cancer Inst. 92:205-16 (2000).

The terms “cell proliferative disorder” and “proliferative disorder” refer to disorders that are associated with some degree of abnormal cell proliferation. In one embodiment, the cell proliferative disorder is cancer. In one embodiment, the cell proliferative disorder is a tumor.

“Tumor,” as used herein, refers to all neoplastic cell growth and proliferation, whether malignant or benign, and all pre-cancerous and cancerous cells and tissues. The terms “cancer”, “cancerous”, “cell proliferative disorder”, “proliferative disorder” and “tumor” are not mutually exclusive as referred to herein.

A “chemotherapeutic agent” is a chemical compound useful in the treatment of cancer. Non-limiting examples of chemotherapeutic agents include alkylating agents such as thiotepa and cyclophosphamide (CYTOXAN®); alkyl sulfonates such as busulfan, improsulfan, and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (especially bullatacin and bullatacinone); delta-9-tetrahydrocannabinol (dronabinol, MARINOL®); beta-lapachone; lapachol; colchicines; betulinic acid; a camptothecin (including the synthetic analogue topotecan (HYCAMTIN®), CPT-11 (irinotecan, CAMPTOSAR®), acetylcamptothecin, scopolectin, and 9-aminocamptothecin); bryostatin; pemetrexed; callystatin; CC-1065 (including its adozelesin, carzelesin and bizelesin synthetic analogues); podophyllotoxin; podophyllinic acid; teniposide; cryptophycins (particularly cryptophycin 1 and cryptophycin 8); dolastatin; duocarmycin (including the synthetic analogues, KW-2189 and CB1-TM1); eleutherobin; pancratistatin; TLK-286; CDP323, an oral alpha-4 integrin inhibitor; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e. g., calicheamicin, especially calicheamicin gammall and calicheamicin omegall (see, e.g., Nicolaou et al., Angew. Chem Intl. Ed. Engl., 33: 183-186 (1994)); dynemicin, including dynemicin A; an esperamicin; as well as neocarzinostatin chromophore and related chromoprotein enediyne antibiotic chromophores), aclacinomysins, actinomycin, authramycin, azaserine, bleomycins, cactinomycin, carabicin, carminomycin, carzinophilin, chromomycinis, dactinomycin, daunorubicin, detorubicin, 6-diazo-5-oxo-L-norleucine, doxorubicin (including ADRIAMYCIN®, morpholino-doxorubicin, cyanomorpholino-doxorubicin, 2-pyrrolino-doxorubicin, doxorubicin HCl liposome injection (DOXIL®) and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate, gemcitabine (GEMZAR®), tegafur (UFTORAL®), capecitabine (XELODA®), an epothilone, and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine, and imatinib (a 2-phenylaminopyrimidine derivative), as well as other c-Kit inhibitors; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elfornithine; elliptinium acetate; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; 2-ethylhydrazide; procarbazine; PSK® polysaccharide complex (JHS Natural Products, Eugene, Oreg.); razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine (ELDISINE®, FILDESIN®); dacarbazine; mannomustine; mitobronitol; mitolactol; pipobroman; gacytosine; arabinoside (“Ara-C”); thiotepa; taxoids, e.g., paclitaxel (TAXOL®), albumin-engineered nanoparticle formulation of paclitaxel (ABRAXANE™), and doxetaxel (TAXOTERE®); chloranbucil; 6-thioguanine; mercaptopurine; methotrexate; platinum analogs such as cisplatin and carboplatin; vinblastine (VELBAN®)); platinum; etoposide (VP-16); ifosfamide; mitoxantrone; vincristine (ONCOVIN®); oxaliplatin; leucovovin; vinorelbine (NAVELBINE®); novantrone; edatrexate; daunomycin; aminopterin; ibandronate; topoisomerase inhibitor RFS 2000; difluorometlhylornithine (DMFO); retinoids such as retinoic acid; pharmaceutically acceptable salts, acids or derivatives of any of the above; as well as combinations of two or more of the above such as CHOP, an abbreviation for a combined therapy of cyclophosphamide, doxorubicin, vincristine, and prednisolone, and FOLFOX, an abbreviation for a treatment regimen with oxaliplatin (ELOXATIN™) combined with 5-FU and leucovovin.

A “chemotherapeutic agent” also includes, without limitation, anti-hormonal agents that act to regulate, reduce, block, or inhibit the effects of hormones that can promote the growth of cancer, and are often in the form of systemic, or whole-body treatment. They may be hormones themselves. Non-limiting examples include anti-estrogens and selective estrogen receptor modulators (SERMs), including, for example, tamoxifen (including NOLVADEX® tamoxifen), raloxifene (EVISTA®), droloxifene, 4-hydroxytamoxifen, trioxifene, keoxifene, LY117018, onapristone, and toremifene (FARESTON®); anti-progesterones; estrogen receptor down-regulators (ERDs); estrogen receptor antagonists such as fulvestrant (FASLODEX®); agents that function to suppress or shut down the ovaries, for example, leutinizing hormone-releasing hormone (LHRH) agonists such as leuprolide acetate (LUPRON® and ELIGARD®), goserelin acetate, buserelin acetate and tripterelin; anti-androgens such as flutamide, nilutamide and bicalutamide; and aromatase inhibitors that inhibit the enzyme aromatase, which regulates estrogen production in the adrenal glands, such as, for example, 4(5)-imidazoles, aminoglutethimide, megestrol acetate (MEGASE®), exemestane (AROMASIN®), formestanie, fadrozole, vorozole (RIVISOR®), letrozole (FEMARA®), and anastrozole (ARIMIDEX®). In addition, such definition of chemotherapeutic agents includes bisphosphonates such as clodronate (for example, BONEFOS® or OSTAC®), etidronate (DIDROCAL®), NE-58095, zoledronic acid/zoledronate (ZOMETA®), alendronate (FOSAMAX®), pamidronate (AREDIA®), tiludronate (SKELID®), or risedronate (ACTONEL®); as well as troxacitabine (a 1,3-dioxolane nucleoside cytosine analog); anti-sense oligonucleotides, particularly those that inhibit expression of genes in signaling pathways implicated in abherant cell proliferation, such as, for example, PKC-alpha, Raf, H-Ras, and epidermal growth factor receptor (EGF-R); vaccines such as THERATOPE® vaccine and gene therapy vaccines, for example, ALLOVECTIN® vaccine, LEUVECTIN® vaccine, and VAXID® vaccine; topoisomerase 1 inhibitor (e.g., LURTOTECAN®); an anti-estrogen such as fulvestrant; a Kit inhibitor such as imatinib or EXEL-0862 (a tyrosine kinase inhibitor); EGFR inhibitor such as erlotinib or cetuximab; an anti-VEGF inhibitor such as bevacizumab; arinotecan; rmRH (e.g., ABARELIX®); lapatinib and lapatinib ditosylate (an ErbB-2 and EGFR dual tyrosine kinase small-molecule inhibitor also known as GW572016); 17AAG (geldanamycin derivative that is a heat shock protein (Hsp) 90 poison), and pharmaceutically acceptable salts, acids or derivatives of any of the above.

By “radiation therapy” is meant the use of directed gamma rays or beta rays to induce sufficient damage to a cell so as to limit its ability to function normally or to destroy the cell altogether. It will be appreciated that there will be many ways known in the art to determine the dosage and duration of treatment. Typical treatments are given as a one-time administration and typical dosages range from 10 to 200 units (Grays) per day.

As used herein, the term “cytokine” refers generically to proteins released by one cell population that act on another cell as intercellular mediators or have an autocrine effect on the cells producing the proteins. Examples of such cytokines include lymphokines, monokines; interleukins (“ILs”) such as IL-1, IL-1α, IL-2, IL-3, IL-4, IL-5, IL-6, IL-7, IL-8, IL-9, IL-10, IL-11, IL-12, IL-13, IL-15, IL-17A-F, IL-18 to IL-29 (such as IL-23), IL-31, including PROLEUKIN® rIL-2; a tumor-necrosis factor such as TNF-α or TNF-β, TGF-β1-3; and other polypeptide factors including leukemia inhibitory factor (“LIF”), ciliary neurotrophic factor (“CNTF”), CNTF-like cytokine (“CLC”), cardiotrophin (“CT”), and kit ligand (“KL”).

As used herein, the term “chemokine” refers to soluble factors (e.g., cytokines) that have the ability to selectively induce chemotaxis and activation of leukocytes. They also trigger processes of angiogenesis, inflammation, wound healing, and tumorigenesis. Example chemokines include IL-8, a human homolog of murine keratinocyte chemoattractant (KC).

“IL-17” as used herein refers to the IL-17 family of cytokines. Unless otherwise specified, a reference to an IL-17 may refer to one or more members of the IL-17 family of cytokines, including, e.g., IL-17A, IL-17B, IL-17C, IL-17D, IL-17E, and IL-17F. In addition, unless otherwise specified, IL-17 may refer to a single IL-17 family cytokine polypeptide or a dimer of IL-17 family cytokine monomers (e.g., IL-17AA, IL-17FF, or IL-17AF).

“IL-17 receptor (IL-17R)” as used herein refers to the family of IL-17 receptors. Unless otherwise specified, a reference to an IL-17 receptor may refer to one or more members of the IL-17 receptor family, including, e.g., IL-17RA, IL-17RB, IL-17RC, IL-17RD, and IL-17RE. In addition, unless otherwise specified, IL-17 receptor may refer to a single IL-17 receptor polypeptide or a dimer of IL-17 receptor monomers (e.g., a receptor complex such as IL-17RA/IL-17RC or IL-17RA/IL-17RB).

The term “IL-17 binding antagonist” as used herein refers to a molecule that decreases, blocks, inhibits, abrogates or interferes with signal transduction resulting from the interaction of an IL-17 cytokine with one or more IL-17 receptors. Examples of types of IL-17 binding antagonists may include a molecule that binds an IL-17 family cytokine and inhibits its interaction with an IL-17 receptor (e.g., an antibody that specifically binds an IL-17 family cytokine, or a soluble polypeptide containing at least one exon of an IL-17 receptor) and/or a molecule that binds an IL-17 receptor and inhibits its interaction with an IL-17 family cytokine (e.g., an antibody that specifically binds an IL-17 receptor). In some embodiments, an IL-17 binding antagonist modulates, blocks, inhibits, reduces, antagonizes, neutralizes or otherwise interferes with the biological activity of an IL-17 cytokine, e.g., IL-17F. IL-17A, and/or the IL-17A/IL-17F heterodimeric complex. In some embodiments, an IL-17 binding antagonist modulates, blocks, inhibits, reduces, antagonizes, neutralizes or otherwise interferes with the biological activity of an IL-17 receptor, e.g., the IL-17RA/IL-17RC receptor complex and/or the IL-17RA/IL-17RB receptor complex. In some embodiments, an IL-17 binding antagonist may include a small molecule.

The phrase “pharmaceutically acceptable salt” as used herein, refers to pharmaceutically acceptable organic or inorganic salts of a compound of the invention. Exemplary salts include, but are not limited, to sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucuronate, saccharate, formate, benzoate, glutamate, methanesulfonate “mesylate”, ethanesulfonate, benzenesulfonate, p-toluenesulfonate, pamoate (i.e., 1,1′-methylene-bis-(2-hydroxy-3-naphthoate)) salts, alkali metal (e.g., sodium and potassium) salts, alkaline earth metal (e.g., magnesium) salts, and ammonium salts. A pharmaceutically acceptable salt may involve the inclusion of another molecule such as an acetate ion, a succinate ion or other counter ion. The counter ion may be any organic or inorganic moiety that stabilizes the charge on the parent compound. Furthermore, a pharmaceutically acceptable salt may have more than one charged atom in its structure. Instances where multiple charged atoms are part of the pharmaceutically acceptable salt can have multiple counter ions. Hence, a pharmaceutically acceptable salt can have one or more charged atoms and/or one or more counter ion.

If the compound of the invention is a base, the desired pharmaceutically acceptable salt may be prepared by any suitable method available in the art, for example, treatment of the free base with an inorganic acid, such as hydrochloric acid, hydrobromic acid, sulfuric acid, nitric acid, methanesulfonic acid, phosphoric acid and the like, or with an organic acid, such as acetic acid, maleic acid, succinic acid, mandelic acid, fumaric acid, malonic acid, pyruvic acid, oxalic acid, glycolic acid, salicylic acid, a pyranosidyl acid, such as glucuronic acid or galacturonic acid, an alpha hydroxy acid, such as citric acid or tartaric acid, an amino acid, such as aspartic acid or glutamic acid, an aromatic acid, such as benzoic acid or cinnamic acid, a sulfonic acid, such as p-toluenesulfonic acid or ethanesulfonic acid, or the like.

If the compound of the invention is an acid, the desired pharmaceutically acceptable salt may be prepared by any suitable method, for example, treatment of the free acid with an inorganic or organic base, such as an amine (primary, secondary or tertiary), an alkali metal hydroxide or alkaline earth metal hydroxide, or the like. Illustrative examples of suitable salts include, but are not limited to, organic salts derived from amino acids, such as glycine and arginine, ammonia, primary, secondary, and tertiary amines, and cyclic amines, such as piperidine, morpholine and piperazine, and inorganic salts derived from sodium, calcium, potassium, magnesium, manganese, iron, copper, zinc, aluminum and lithium.

The phrase “pharmaceutically acceptable” indicates that the substance or composition must be compatible chemically and/or toxicologically, with the other ingredients comprising a formulation, and/or the mammal being treated therewith.

The term “detection” includes any means of detecting, including direct and indirect detection.

The term “biomarker” as used herein refers to an indicator, e.g., predictive, diagnostic, and/or prognostic, which can be detected in a sample. The biomarker may serve as an indicator of a particular subtype of a disease or disorder (e.g., cancer) characterized by certain, molecular, pathological, histological, and/or clinical features. In some embodiments, a biomarker is a gene. Biomarkers include, but are not limited to, polynucleotides (e.g., DNA, and/or RNA), polynucleotide copy number alterations (e.g., DNA copy numbers), polypeptides, polypeptide and polynucleotide modifications (e.g. posttranslational modifications), carbohydrates, and/or glycolipid % ased molecular markers.

The terms “biomarker signature,” “signature,” “biomarker expression signature,” or “expression signature” are used interchangeably herein and refer to one or a combination of biomarkers whose expression is an indicator, e.g., predictive, diagnostic, and/or prognostic. The biomarker signature may serve as an indicator of a particular subtype of a disease or disorder (e.g., cancer) characterized by certain molecular, pathological, histological, and/or clinical features. In some embodiments, the biomarker signature is a “gene signature.” The term “gene signature” is used interchangeably with “gene expression signature” and refers to one or a combination of polynucleotides whose expression is an indicator, e.g., predictive, diagnostic, and/or prognostic. In some embodiments, the biomarker signature is a “protein signature.” The term “protein signature” is used interchangeably with “protein expression signature” and refers to one or a combination of polypeptides whose expression is an indicator, e.g., predictive, diagnostic, and/or prognostic.

The “amount” or “level” of a biomarker associated with an increased clinical benefit to an individual is a detectable level in a biological sample. These can be measured by methods known to one skilled in the art and also disclosed herein. The expression level or amount of biomarker assessed can be used to determine the response to the treatment.

The terms “level of expression” or “expression level” in general are used interchangeably and generally refer to the amount of a biomarker in a biological sample. “Expression” generally refers to the process by which information (e.g., geneencoded and/or epigenetic) is converted into the structures present and operating in the cell. Therefore, as used herein, “expression” may refer to transcription into a polynucleotide, translation into a polypeptide, or even polynucleotide and/or polypeptide modifications (e.g., posttranslational modification of a polypeptide). Fragments of the transcribed polynucleotide, the translated polypeptide, or polynucleotide and/or polypeptide modifications (e.g., posttranslational modification of a polypeptide) shall also be regarded as expressed whether they originate from a transcript generated by alternative splicing or a degraded transcript, or from a posttranslational processing of the polypeptide, e.g., by proteolysis. “Expressed genes” include those that are transcribed into a polynucleotide as mRNA and then translated into a polypeptide, and also those that are transcribed into RNA but not translated into a polypeptide (for example, transfer and ribosomal RNAs).

“Elevated expression,” “elevated expression levels,” or “elevated levels” refers to an increased expression or increased levels of a biomarker in an individual relative to a control, such as an individual or individuals who are not suffering from the disease or disorder (e.g., cancer) or an internal control (e.g., housekeeping biomarker).

“Reduced expression,” “reduced expression levels,” or “reduced levels” refers to a decrease expression or decreased levels of a biomarker in an individual relative to a control, such as an individual or individuals who are not suffering from the disease or disorder (e.g., cancer) or an internal control (e.g., housekeeping biomarker). In some embodiments, reduced expression is little or no expression.

The term “housekeeping biomarker” refers to a biomarker or group of biomarkers (e.g., polynucleotides and/or polypeptides) which are typically similarly present in all cell types. In some embodiments, the housekeeping biomarker is a “housekeeping gene.” A “housekeeping gene” refers herein to a gene or group of genes which encode proteins whose activities are essential for the maintenance of cell function and which are typically similarly present in all cell types.

“Amplification,” as used herein generally refers to the process of producing multiple copies of a desired sequence. “Multiple copies” mean at least two copies. A “copy” does not necessarily mean perfect sequence complementarity or identity to the template sequence. For example, copies can include nucleotide analogs such as deoxyinosine, intentional sequence alterations (such as sequence alterations introduced through a primer comprising a sequence that is hybridizable, but not complementary, to the template), and/or sequence errors that occur during amplification.

The term “multiplex-PCR” refers to a single PCR reaction carried out on nucleic acid obtained from a single source (e.g., an individual) using more than one primer set for the purpose of amplifying two or more DNA sequences in a single reaction.

“Stringency” of hybridization reactions is readily determinable by one of ordinary skill in the art, and generally is an empirical calculation dependent upon probe length, washing temperature, and salt concentration. In general, longer probes require higher temperatures for proper annealing, while shorter probes need lower temperatures. Hybridization generally depends on the ability of denatured DNA to reanneal when complementary strands are present in an environment below their melting temperature. The higher the degree of desired homology between the probe and hybridizable sequence, the higher the relative temperature which can be used. As a result, it follows that higher relative temperatures would tend to make the reaction conditions more stringent, while lower temperatures less so. For additional details and explanation of stringency of hybridization reactions, see Ausubel et al., Current Protocols in Molecular Biology, Wiley Interscience Publishers, (1995).

“Stringent conditions” or “high stringency conditions”, as defined herein, can be identified by those that: (1) employ low ionic strength and high temperature for washing, for example 0.015 M sodium chloride/0.0015 M sodium citrate/0.1% sodium dodecyl sulfate at 50° C.; (2) employ during hybridization a denaturing agent, such as formamide, for example, 50% (v/v) formamide with 0.1% bovine serum albumin/0.1% Ficoll/0.1% polyvinylpyrrolidone/50 mM sodium phosphate buffer at pH 6.5 with 750 mM sodium chloride, 75 mM sodium citrate at 42° C.; or (3) overnight hybridization in a solution that employs 50% formamide, 5×SSC (0.75 M NaCl, 0.075 M sodium citrate), 50 mM sodium phosphate (pH 6.8), 0.1% sodium pyrophosphate, 5×Denhardt's solution, sonicated salmon sperm DNA (50 μg/mL), 0.1% SDS, and 10% dextran sulfate at 42° C., with a 10 minute wash at 42° C. in 0.2×SSC (sodium chloride/sodium citrate) followed by a 10 minute high-stringency wash consisting of 0.1×SSC containing EDTA at 55° C.

“Moderately stringent conditions” can be identified as described by Sambrook et al., Molecular Cloning: A Laboratory Manual, New York: Cold Spring Harbor Press, 1989, and include the use of washing solution and hybridization conditions (e.g., temperature, ionic strength and % SDS) less stringent that those described above. An example of moderately stringent conditions is overnight incubation at 37° C. in a solution comprising: 20% formamide, 5×SSC (150 mM NaCl, 15 mM trisodium citrate), 50 mM sodium phosphate (pH 7.6), 5×Denhardt's solution, 10% dextran sulfate, and 20 mg/ml denatured sheared salmon sperm DNA, followed by washing the filters in 1×SSC at about 37-50° C. The skilled artisan will recognize how to adjust the temperature, ionic strength, etc. as necessary to accommodate factors such as probe length and the like.

The technique of “polymerase chain reaction” or “PCR” as used herein generally refers to a procedure wherein minute amounts of a specific piece of nucleic acid, RNA and/or DNA, are amplified as described in U.S. Pat. No. 4,683,195 issued 28 Jul. 1987. Generally, sequence information from the ends of the region of interest or beyond needs to be available, such that oligonucleotide primers can be designed; these primers will be identical or similar in sequence to opposite strands of the template to be amplified. The 5′ terminal nucleotides of the two primers may coincide with the ends of the amplified material. PCR can be used to amplify specific RNA sequences, specific DNA sequences from total genomic DNA, and cDNA transcribed from total cellular RNA, bacteriophage or plasmid sequences, etc. See generally Mullis et al., Cold Spring Harbor Symp. Quant. Biol., 51: 263 (1987); Erlich, ed., PCR Technology, (Stockton Press, N Y, 1989). As used herein, PCR is considered to be one, but not the only, example of a nucleic acid polymerase reaction method for amplifying a nucleic acid test sample, comprising the use of a known nucleic acid (DNA or RNA) as a primer and utilizes a nucleic acid polymerase to amplify or generate a specific piece of nucleic acid or to amplify or generate a specific piece of nucleic acid which is complementary to a particular nucleic acid.

“Quantitative real time polymerase chain reaction” or “qRT-PCR” refers to a form of PCR wherein the amount of PCR product is measured at each step in a PCR reaction. This technique has been described in various publications including Cronin et al., Am. J. Pathol. 164(1):35-42 (2004); and Ma et al., Cancer Cell 5:607-616 (2004).

The term “microarray” refers to an ordered arrangement of hybridizable array elements, preferably polynucleotide probes, on a substrate.

The term “oligonucleotide” refers to a relatively short polynucleotide, including, without limitation, single-stranded deoxyribonucleotides, single- or double-stranded ribonucleotides, RNA:DNA hybrids and double-stranded DNAs. Oligonucleotides, such as single-stranded DNA probe oligonucleotides, are often synthesized by chemical methods, for example using automated oligonucleotide synthesizers that are commercially available. However, oligonucleotides can be made by a variety of other methods, including in vitro recombinant DNA-mediated techniques and by expression of DNAs in cells and organisms.

The term “polynucleotide,” when used in singular or plural, generally refers to any polyribonucleotide or polydeoxyribonucleotide, which may be unmodified RNA or DNA or modified RNA or DNA. Thus, for instance, polynucleotides as defined herein include, without limitation, single- and double-stranded DNA, DNA including single- and double-stranded regions, single- and double-stranded RNA, and RNA including single- and double-stranded regions, hybrid molecules comprising DNA and RNA that may be single-stranded or, more typically, double-stranded or include single- and double-stranded regions. In addition, the term “polynucleotide” as used herein refers to triple-stranded regions comprising RNA or DNA or both RNA and DNA. The strands in such regions may be from the same molecule or from different molecules. The regions may include all of one or more of the molecules, but more typically involve only a region of some of the molecules. One of the molecules of a triple-helical region often is an oligonucleotide. The term “polynucleotide” specifically includes cDNAs. The term includes DNAs (including cDNAs) and RNAs that contain one or more modified bases. Thus, DNAs or RNAs with backbones modified for stability or for other reasons are “polynucleotides” as that term is intended herein. Moreover, DNAs or RNAs comprising unusual bases, such as inosine, or modified bases, such as tritiated bases, are included within the term “polynucleotides” as defined herein. In general, the term “polynucleotide” embraces all chemically, enzymatically and/or metabolically modified forms of unmodified polynucleotides, as well as the chemical forms of DNA and RNA characteristic of viruses and cells, including simple and complex cells.

The term “diagnosis” is used herein to refer to the identification or classification of a molecular or pathological state, disease or condition (e.g., cancer). For example, “diagnosis” may refer to identification of a particular type of cancer. “Diagnosis” may also refer to the classification of a particular subtype of cancer, e.g., by histopathological criteria, or by molecular features (e.g., a subtype characterized by expression of one or a combination of biomarkers (e.g., particular genes or proteins encoded by said genes)).

The term “aiding diagnosis” is used herein to refer to methods that assist in making a clinical determination regarding the presence, or nature, of a particular type of symptom or condition of a disease or disorder (e.g., cancer). For example, a method of aiding diagnosis of a disease or condition (e.g., cancer) can comprise measuring certain biomarkers in a biological sample from an individual.

The term “sample,” as used herein, refers to a composition that is obtained or derived from a subject and/or individual of interest that contains a cellular and/or other molecular entity that is to be characterized and/or identified, for example based on physical, biochemical, chemical and/or physiological characteristics. For example, the phrase “disease sample” and variations thereof refers to any sample obtained from a subject of interest that would be expected or is known to contain the cellular and/or molecular entity that is to be characterized. Samples include, but are not limited to, primary or cultured cells or cell lines, cell supernatants, cell lysates, platelets, serum, plasma, vitreous fluid, lymph fluid, synovial fluid, follicular fluid, seminal fluid, amniotic fluid, milk, whole blood, blood-derived cells, urine, cerebrospinal fluid, saliva, sputum, tears, perspiration, mucus, tumor lysates, and tissue culture medium, tissue extracts such as homogenized tissue, tumor tissue, cellular extracts, and combinations thereof.

By “tissue sample” or “cell sample” is meant a collection of similar cells obtained from a tissue of a subject or individual. The source of the tissue or cell sample may be solid tissue as from a fresh, frozen and/or preserved organ, tissue sample, biopsy, and/or aspirate; blood or any blood constituents such as plasma; bodily fluids such as cerebral spinal fluid, amniotic fluid, peritoneal fluid, or interstitial fluid; cells from any time in gestation or development of the subject. The tissue sample may also be primary or cultured cells or cell lines. Optionally, the tissue or cell sample is obtained from a disease tissue/organ. The tissue sample may contain compounds which are not naturally intermixed with the tissue in nature such as preservatives, anticoagulants, buffers, fixatives, nutrients, antibiotics, or the like.

For the purposes herein a “section” of a tissue sample is meant a single part or piece of a tissue sample, e.g. a thin slice of tissue or cells cut from a tissue sample. It is understood that multiple sections of tissue samples may be taken and subjected to analysis, provided that it is understood that the same section of tissue sample may be analyzed at both morphological and molecular levels, or analyzed with respect to both polypeptides and polynucleotides.

By “correlate” or “correlating” is meant comparing, in any way, the performance and/or results of a first analysis or protocol with the performance and/or results of a second analysis or protocol. For example, one may use the results of a first analysis or protocol in carrying out a second protocols and/or one may use the results of a first analysis or protocol to determine whether a second analysis or protocol should be performed. With respect to the embodiment of polypeptide analysis or protocol, one may use the results of the polypeptide expression analysis or protocol to determine whether a specific therapeutic regimen should be performed. With respect to the embodiment of polynucleotide analysis or protocol, one may use the results of the polynucleotide expression analysis or protocol to determine whether a specific therapeutic regimen should be performed.

“Individual response” or “response” can be assessed using any endPoint indicating a benefit to the individual, including, without limitation, (1) inhibition, to some extent, of disease progression (e.g., cancer progression), including slowing down and complete arrest; (2) a reduction in tumor size; (3) inhibition (i.e., reduction, slowing down or complete stopping) of cancer cell infiltration into adjacent peripheral organs and/or tissues; (4) inhibition (i.e. reduction, slowing down or complete stopping) of metastasis; (5) relief, to some extent, of one or more symptoms associated with the disease or disorder (e.g., cancer); (6) increase or extend in the length of survival, including overall survival and progression free survival; and/or (9) decreased mortality at a given Point of time following treatment.

An “effective response” of a patient or a patient's “responsiveness” to treatment with a medicament and similar wording refers to the clinical or therapeutic benefit imparted to a patient at risk for, or suffering from, a disease or disorder, such as cancer. In one embodiment, such benefit includes any one or more of: extending survival (including overall survival and progression free survival); resulting in an objective response (including a complete response or a partial response); or improving signs or symptoms of cancer. In one embodiment, the biomarker (e.g., PD-L1 expression, for example, as determined using IHC) is used to identify the patient who is predicted to have an increase likelihood of being responsive to treatment with a medicament (e.g., anti-PDL1 antibody), relative to a patient who does not express the biomarker. In one embodiment, the biomarker (e.g., PD-L1 expression, for example, as determined using IHC) is used to identify the patient who is predicted to have an increase likelihood of being responsive to treatment with a medicament (e.g., anti-PDL1 antibody), relative to a patient who does not express the biomarker at the same level. In one embodiment, the presence of the biomarker is used to identify a patient who is more likely to respond to treatment with a medicament, relative to a patient that does not have the presence of the biomarker. In another embodiment, the presence of the biomarker is used to determine that a patient will have an increase likelihood of benefit from treatment with a medicament, relative to a patient that does not have the presence of the biomarker.

By “extending survival” is meant increasing overall or progression free survival in a treated patient relative to an untreated patient (i.e. relative to a patient not treated with the medicament), or relative to a patient who does not express a biomarker at the designated level, and/or relative to a patient treated with an approved anti-tumor agent. An objective response refers to a measurable response, including complete response (CR) or partial response (PR).

### III. PD-1 Axis Binding Antagonists

Provided herein are methods for treating or delaying progression of cancer in an individual comprising administering to the individual an effective amount of a PD-1 axis binding antagonist and an IL-17 binding antagonist. For example, a PD-1 axis binding antagonist includes a PD-1 binding antagonist, a PDL1 binding antagonist and a PDL2 binding antagonist. Alternative names for “PD-1” include CD279 and SLEB2. Alternative names for “PDL1” include B7-H1, B7-4, CD274, and B7-H. Alternative names for “PDL2” include B7-DC, Btdc, and CD273. In some embodiments, PD-1, PDL1, and PDL2 are human PD-1, PDL1 and PDL2.

In some embodiments, the PD-1 binding antagonist is a molecule that inhibits the binding of PD-1 to its ligand binding partners. In a specific aspect the PD-1 ligand binding partners are PDL1 and/or PDL2. In another embodiment, a PDL1 binding antagonist is a molecule that inhibits the binding of PDL1 to its binding partners. In a specific aspect, PDL1 binding partners are PD-1 and/or B7-1. In another embodiment, the PDL2 binding antagonist is a molecule that inhibits the binding of PDL2 to its binding partners. In a specific aspect, a PDL2 binding partner is PD-1. The antagonist may be an antibody, an antigen binding fragment thereof, an immunoadhesin, a fusion protein, or oligopeptide.

In some embodiments, the PD-1 binding antagonist is an anti-PD-1 antibody (e.g., a human antibody, a humanized antibody, or a chimeric antibody). In some embodiments, the anti-PD-1 antibody is selected from the group consisting of nivolumab, pembrolizumab, and CT-011. In some embodiments, the anti-PD-1 antibody is selected from the group consisting of nivolumab, pembrolizumab, CT-011, MEDI-0680 (AMP-514), PDR001, REGN2810, BGB-108, and BGB-A317. In some embodiments, the PD-1 binding antagonist is an immunoadhesin (e.g., an immunoadhesin comprising an extracellular or PD-1 binding portion of PDL1 or PDL2 fused to a constant region (e.g., an Fc region of an immunoglobulin sequence). In some embodiments, the PD-1 binding antagonist is AMP-224. Nivolumab, also known as MDX-1106-04, MDX-1106, ONO-4538, BMS-936558, and OPDIVO®, is an anti-PD-1 antibody described in WO2006/121168. Pembrolizumab, also known as MK-3475, Merck 3475, lambrolizumab, KEYTRUDA®, and SCH-900475, is an anti-PD-1 antibody described in WO2009/114335. CT-011, also known as hBAT, hBAT-1, and pidilizumab, is an anti-PD-1 antibody described in WO2009/101611. AMP-224, also known as B7-DCIg, is a PDL2-Fc fusion soluble receptor described in WO2010/027827 and WO2011/066342.

In some embodiments, the anti-PD-1 antibody is nivolumab (CAS Registry Number: 946414-94-4). In a still further embodiment, provided is an isolated anti-PD-1 antibody comprising a heavy chain variable region comprising the heavy chain variable region amino acid sequence from SEQ ID NO:22 and/or a light chain variable region comprising the light chain variable region amino acid sequence from SEQ ID NO:23. In a still further embodiment, provided is an isolated anti-PD-1 antibody comprising a heavy chain and/or a light chain sequence, wherein:

(a) the heavy chain sequence has at least 85%, at least 90%, at least 91%, at least 92%, at least 93%, at least 94%, at least 95%, at least 96%, at least 97%, at least 98%, at least 99% or 100% sequence identity to the heavy chain sequence: QVQLVESGGGVVQPGRSLRLDCKASGITFSNSGMHWVRQAPGKGLEWVAVIWY DGSKRYYADSVKGRFTISRDNSKNTLFLQMNSLRAEDTAVYYCATNDDYWGQGTLVT VSSASTKGPSVFPLAPCSRSTSESTAALGCLVKDYFPEPVTVSWNSGALTSGVHTFPAVL QSSGLYSLSSVVTVPSSSLGTKTYTCNVDHKPSNTKVDKRVESKYGPPCPPCPAPEFLGG PSVFLFPPKPKDTLMISRTPEVTCVVVDVSQEDPEVQFNWYVDGVEVHNAKTKPREEQF NSTYRVVSVLTVLHQDWLNGKEYKCKVSNKGLPSSIEKTISKAKGQPREPQVYTLPPSQ EEMTKNQVSLTCLVKGFYPSDIAVEWESNGQPENNYKTTPPVLDSDGSFFLYSRLTVDK SRWQEGNVFSCSVMHEALHNHYTQKSLSLSLGK (SEQ ID NO:22), or

(b) the light chain sequences has at least 85%, at least 90%, at least 91%, at least 92%, at least 93%, at least 94%, at least 95%, at least 96%, at least 97%, at least 98%, at least 99% or 100% sequence identity to the light chain sequence:

(SEQ ID NO: 23)
EIVLTQSPATLSLSPGERATLSCRASQSVSSYLAWYQQKPGQAPRLLIY
DASNRATGIPARFSGSGSGTDFTLTISSLEPEDFAVYYCQQSSNWPRTF
GQGTKVEIKRTVAAPSVFIFPPSDEQLKSGTASVVCLLNNFYPREAKVQ
WKVDNALQSGNSQESVTEQDSKDSTYSLSSTLTLSKADYEKHKVYACEV
THQGLSSPVTKSFNRGEC.

In some embodiments, the anti-PD-1 antibody is pembrolizumab (CAS Registry Number: 1374853-91-4). In a still further embodiment, provided is an isolated anti-PD-1 antibody comprising a heavy chain variable region comprising the heavy chain variable region amino acid sequence from SEQ ID NO:62 and/or a light chain variable region comprising the light chain variable region amino acid sequence from SEQ ID NO:63. In a still further embodiment, provided is an isolated anti-PD-1 antibody comprising a heavy chain and/or a light chain sequence, wherein:

(a) the heavy chain sequence has at least 85%, at least 90%, at least 91%, at least 92%, at least 93%, at least 94%, at least 95%, at least 96%, at least 97%, at least 98%, at least 99% or 100% sequence identity to the heavy chain sequence: QVQLVQSGVE VKKPGASVKV SCKASGYTFT NYYMYWVRQA PGQGLEWMGG INPSNGGTNF NEKFKNRVTL TTDSSTTTAY MELKSLOFDD TAVYYCARRDYRFDMGFDYW GOGTTVTVSS ASTKGPSVFP LAPCSRSTSE STAALGCLVKDYFPEPVTVS WNSGALTSGV HTFPAVLQSS GLYSLSSVVT VPSSSLGTKTYTCNVDHKPS NTKVDKRVES KYGPPCPPCP APEFLGGPSV FLFPPKPKDTLMISRTPEVT CVVVDVSQED PEVQFNWYVD GVEVHNAKTK PREEQFNSTYRVVSVLTVLH QDWLNGKEYK CKVSNKGLPS SIEKTISKAK GQPREPQVYTLPPSQEEMTK NQVSLTCLVK GFYPSDIAVE WESNGQPENN YKTTPPVLDSDGSFFLYSRL TVDKSRWQEG NVFSCSVMHE ALHNHYTQKS LSLSLGK (SEQ ID NO:62), or

(b) the light chain sequences has at least 85%, at least 90%, at least 91%, at least 92%, at least 93%, at least 94%, at least 95%, at least 96%, at least 97%, at least 98%, at least 99% or 100% sequence identity to the light chain sequence: EIVLTQSPAT LSLSPGERATLSCRASKGVSTSGYSYLHWYQQKPGQAPRL LIYLASYLES GVPARFSGSG SGTDFTLTISSLEPEDFAVYYCQHSRDLPLTFGGGTKVEI KRTVAAPSVF IFPPSDEQLK SGTASVVCLL NNFYPREAKVQWKVDNALQS GNSQESVTEQ DSKDSTYSLSSTLTLSKADYEKHKVYACEVTHQGLSSPVT KSFNRGEC (SEQ ID NO:63).

In some embodiments, the PDL1 binding antagonist is anti-PDL1 antibody. In some embodiments, the anti-PDL1 binding antagonist is selected from the group consisting of YW243.55.570, MPDL3280A, MDX-1105, and MEDI4736. In some embodiments, the anti-PDL1 binding antagonist is selected from the group consisting of YW243.55.570, MPDL3280A (also known as atezolizumab), MDX-1105, MEDI4736 (also known as durvalumab), and MSB0010718C (also known as avelumab). MDX-1105, also known as BMS-936559, is an anti-PDL1 antibody described in WO2007/005874. Antibody YW243.55.570 (heavy and light chain variable region sequences shown in SEQ ID Nos. 20 and 21, respectively) is an anti-PDL1 described in WO 2010/077634 A1. MEDI4736 is an anti-PDL1 antibody described in WO2011/066389 and US2013/034559.

Examples of anti-PDL1 antibodies useful for the methods of this invention, and methods for making thereof are described in PCT patent application WO 2010/077634 A1 and U.S. Pat. No. 8,217,149, which are incorporated herein by reference.

In some embodiments, the PD-1 axis binding antagonist is an anti-PDL1 antibody. In some embodiments, the anti-PDL1 antibody is capable of inhibiting binding between PDL1 and PD-1 and/or between PDL1 and B7-1. In some embodiments, the anti-PDL1 antibody is a monoclonal antibody. In some embodiments, the anti-PDL1 antibody is an antibody fragment selected from the group consisting of Fab, Fab′-SH, Fv, scFv, and (Fab′)₂ fragments. In some embodiments, the anti-PDL1 antibody is a humanized antibody. In some embodiments, the anti-PDL1 antibody is a human antibody.

The anti-PDL1 antibodies useful in this invention, including compositions containing such antibodies, such as those described in WO 2010/077634 A1, may be used in combination with an IL-17 binding antagonist to treat cancer. In some embodiments, the anti-PDL1 antibody comprises a heavy chain variable region comprising the amino acid sequence of SEQ ID NO:20 and a light chain variable region comprising the amino acid sequence of SEQ ID NO:21.

In one embodiment, the anti-PDL1 antibody contains a heavy chain variable region polypeptide comprising an HVR-H1, HVR-H2 and HVR-H3 sequence, wherein:

(SEQ ID NO: 1)
(a) the HVR-H1 sequence is GFTFSX₁SWIH;
(SEQ ID NO: 2)
(b) the HVR-H2 sequence is AWIX₂PYGGSX₃YYADSVKG;
(SEQ ID NO: 3)
(c) the HVR-H3 sequence is RHWPGGFDY;

further wherein: X₁ is D or G; X₂ is S or L; X₃ is T or S.

In one specific aspect, X₁ is D; X₂ is S and X₃ is T. In another aspect, the polypeptide further comprises variable region heavy chain framework sequences juxtaposed between the HVRs according to the formula: (HC-FR1)-(HVR-H1)-(HC-FR2)-(HVR-H2)-(HC-FR3)-(HVR-H3)-(HC-FR4). In yet another aspect, the framework sequences are derived from human consensus framework sequences. In a further aspect, the framework sequences are VH subgroup III consensus framework. In a still further aspect, at least one of the framework sequences is the following:

(SEQ ID NO: 4)
HC-FR1 is EVQLVESGGGLVQPGGSLRLSCAAS
(SEQ ID NO: 5)
HC-FR2 is WVRQAPGKGLEWV
(SEQ ID NO: 6)
HC-FR3 is RFTISADTSKNTAYLQMNSLRAEDTAVYYCAR
(SEQ ID NO: 7)
HC-FR4 is WGQGTLVTVSA.

In a still further aspect, the heavy chain polypeptide is further combined with a variable region light chain comprising an HVR-L1, HVR-L2 and HVR-L3, wherein:

(SEQ ID NO: 8)
(a) the HVR-L1 sequence is RASQX₄X₅X₆TX₇X₈A
(SEQ ID NO: 9);
(b) the HVR-L2 sequence is SASX₉LX₁₀S,
(SEQ ID NO: 10)
(c) the HVR-L3 sequence is QQX₁₁X₁₂X₁₃X₁₄PX₁₅T;

further wherein: X₄ is D or V; X₅ is V or I; X₆ is S or N; X₇ is A or F; X₈ is V or L; X₉ is F or T; X₁₀ is Y or A; X₁₁ is Y, G, F, or S; X₁₂ is L, Y, F or W; X₁₃ is Y, N, A, T, G, F or I; X₁₄ is H, V, P, T or I; X₁₅ is A, W, R, P or T.

In a still further aspect, X₄ is D; X₅ is V; X₆ is 5; X₇ is A; X₈ is V; X₉ is F; X₁₀ is Y; X₁₁ is Y; X₁₂ is L; X₁₃ is Y; X₁₄ is H; X₁₅ is A. In a still further aspect, the light chain further comprises variable region light chain framework sequences juxtaposed between the HVRs according to the formula: (LC-FR1)-(HVR-L1)-(LC-FR2)-(HVR-L2)-(LC-FR3)-(HVR-L3)-(LC-FR4). In a still further aspect, the framework sequences are derived from human consensus framework sequences. In a still further aspect, the framework sequences are VL kappa I consensus framework. In a still further aspect, at least one of the framework sequence is the following:

(SEQ ID NO: 11)
LC-FR1 is DIQMTQSPSSLSASVGDRVTITC
(SEQ ID NO: 12)
LC-FR2 is WYQQKPGKAPKLLIY
(SEQ ID NO: 13)
LC-FR3 is GVPSRFSGSGSGTDFTLTISSLQPEDFATYYC
(SEQ ID NO: 14)
LC-FR4 is FGQGTKVEIKR.

In another embodiment, provided is an isolated anti-PDL1 antibody or antigen binding fragment comprising a heavy chain and a light chain variable region sequence, wherein:

(a) the heavy chain comprises and HVR-H1, HVR-H2 and HVR-H3, wherein further:

(SEQ ID NO: 1)

| | |
|---|---|
| (i) | the HVR-H1 sequence is GFTFSX₁SWIH; |

(SEQ ID NO: 2)

| | |
|---|---|
| (ii) | the HVR-H2 sequence is AWIX₂PYGGSX₃YYADSVKG |

(SEQ ID NO: 3)

| | |
|---|---|
| (iii) | the HVR-H3 sequence is RHWPGGFDY, |
| and | |

(b) the light chain comprises and HVR-L1, HVR-L2 and HVR-L3, wherein further:

(SEQ ID NO: 8)

| | |
|---|---|
| (i) | the HVR-L1 sequence is RASQX₄X₅X₆TX₇X₈A |

(SEQ ID NO: 9)

| | |
|---|---|
| (ii) | the HVR-L2 sequence is SASX₉LX₁₀S; |
| and | |

(SEQ ID NO: 10)

| | |
|---|---|
| (iii) | the HVR-L3 sequence is QQX₁₁X₁₂X₁₃X₁₄PX₁₅T; |

Further wherein: X₁ is D or G; X₂ is S or L; X₃ is T or S; X₄ is D or V; X₅ is V or I; X₆ is S or N; X₇ is A or F; X₈ is V or L; X₉ is F or T; X₁₀ is Y or A; X₁₁ is Y, G, F, or S; X₁₂ is L, Y, F or W; X₁₃ is Y, N, A, T, G, F or I; X₁₄ is H, V, P, T or I; X₁₅ is A, W, R, P or T.

In a specific aspect, X₁ is D; X₂ is S and X₃ is T. In another aspect, X₄ is D; X₅ is V; X₆ is S; X₇ is A; X₈ is V; X₉ is F; X₁₀ is Y; X₁₁ is Y; X₁₂ is L; X₁₃ is Y; X₁₄ is H; X₁₅ is A. In yet another aspect, X₁ is D; X₂ is S and X₃ is T, X₄ is D; X₅ is V; X₆ is S; X₇ is A; X₈ is V; X₉ is F; X₁₀ is Y; X₁₁ is Y; X₁₂ is L; X₁₃ is Y; X₁₄ is H and X₁₅ is A.

In a further aspect, the heavy chain variable region comprises one or more framework sequences juxtaposed between the HVRs as: (HC-FR1)-(HVR-H1)-(HC-FR2)-(HVR-H2)-(HC-FR3)-(HVR-H3)-(HC-FR4), and the light chain variable regions comprises one or more framework sequences juxtaposed between the HVRs as: (LC-FR1)-(HVR-L1)-(LC-FR2)-(HVR-L2)-(LC-FR3)-(HVR-L3)-(LC-FR4). In a still further aspect, the framework sequences are derived from human consensus framework sequences. In a still further aspect, the heavy chain framework sequences are derived from a Kabat subgroup I, II, or III sequence. In a still further aspect, the heavy chain framework sequence is a VH subgroup III consensus framework. In a still further aspect, one or more of the heavy chain framework sequences is the following:

| (SEQ ID NO: 4) | |
|---|---|
| HC-FR1 | EVQLVESGGGLVQPGGSLRLSCAAS |
| (SEQ ID NO: 5) | |
| HC-FR2 | WVRQAPGKGLEWV |
| (SEQ ID NO: 6) | |
| HC-FR3 | RFTISADTSKNTAYLQMNSLRAEDTAVYYCAR |
| (SEQ ID NO: 7) | |
| HC-FR4 | WGQGTLVTVSA. |

In a still further aspect, the light chain framework sequences are derived from a Kabat kappa I, II, II or IV subgroup sequence. In a still further aspect, the light chain framework sequences are VL kappa I consensus framework. In a still further aspect, one or more of the light chain framework sequences is the following:

| (SEQ ID NO: 11) | |
|---|---|
| LC-FR1 | DIQMTQSPSSLSASVGDRVTITC |
| (SEQ ID NO: 12) | |
| LC-FR2 | WYQQKPGKAPKLLIY |
| (SEQ ID NO: 13) | |
| LC-FR3 | GVPSRFSGSGSGTDFTLTISSLQPEDFATYYC |
| (SEQ ID NO: 14) | |
| LC-FR4 | FGQGTKVEIKR. |

In a still further specific aspect, the antibody further comprises a human or murine constant region. In a still further aspect, the human constant region is selected from the group consisting of IgG1, IgG2, IgG2, IgG3, IgG4. In a still further specific aspect, the human constant region is IgG1. In a still further aspect, the murine constant region is selected from the group consisting of IgG1, IgG2A, IgG2B, IgG3. In a still further aspect, the murine constant region if IgG2A. In a still further specific aspect, the antibody has reduced or minimal effector function. In a still further specific aspect the minimal effector function results from an “effector-less Fc mutation” or aglycosylation. In still a further embodiment, the effector-less Fc mutation is an N297A or D265A/N297A substitution in the constant region.

In yet another embodiment, provided is an anti-PDL1 antibody comprising a heavy chain and a light chain variable region sequence, wherein:
(a) the heavy chain further comprises and HVR-H1, HVR-H2 and an HVR-H3 sequence having at least 85% sequence identity to GFTFSDSWIH (SEQ ID NO:15), AWISPYGGSTYYADSVKG (SEQ ID NO:16) and RHWPGGFDY (SEQ ID NO:3), respectively, or
(b) the light chain further comprises an HVR-L1, HVR-L2 and an HVR-L3 sequence having at least 85% sequence identity to RASQDVSTAVA (SEQ ID NO:17), SASFLYS (SEQ ID NO:18) and QQYLYHPAT (SEQ ID NO:19), respectively.

In a specific aspect, the sequence identity is 86%, 87%, 88%, 89%, 90%, 91%, 92%, 93%, 94%, 95%, 96%, 97%, 98%, 99% or 100%. In another aspect, the heavy chain variable region comprises one or more framework sequences juxtaposed between the HVRs as: (HC-FR1)-(HVR-H1)-(HC-FR2)-(HVR-H2)-(HC-FR3)-(HVR-H3)-(HC-FR4), and the light chain variable regions comprises one or more framework sequences juxtaposed between the HVRs as: (LC-FR1)-(HVR-L1)-(LC-FR2)-(HVR-L2)-(LC-FR3)-(HVR-L3)-(LC-FR4). In yet another aspect, the framework sequences are derived from human consensus framework sequences. In a still further aspect, the heavy chain framework sequences are derived from a Kabat subgroup I, II, or III sequence. In a still further aspect, the heavy chain framework sequence is a VH subgroup III consensus framework. In a still further aspect, one or more of the heavy chain framework sequences is the following:

| (SEQ ID NO: 4) | |
|---|---|
| HC-FR1 | EVQLVESGGGLVQPGGSLRLSCAAS |
| (SEQ ID NO: 5) | |
| HC-FR2 | WVRQAPGKGLEWV |
| (SEQ ID NO: 6) | |
| HC-FR3 | RFTISADTSKNTAYLQMNSLRAEDTAVYYCAR |
| (SEQ ID NO: 7) | |
| HC-FR4 | WGQGTLVTVSA. |

In a still further aspect, the light chain framework sequences are derived from a Kabat kappa I, II, II or IV subgroup sequence. In a still further aspect, the light chain framework sequences are VL kappa I consensus framework. In a still further aspect, one or more of the light chain framework sequences is the following:

| (SEQ ID NO: 11) | |
|---|---|
| LC-FR1 | DIQMTQSPSSLSASVGDRVTITC |
| (SEQ ID NO: 12) | |
| LC-FR2 | WYQQKPGKAPKLLIY |
| (SEQ ID NO: 13) | |
| LC-FR3 | GVPSRFSGSGSGTDFTLTISSLQPEDFATYYC |
| (SEQ ID NO: 14) | |
| LC-FR4 | FGQGTKVEIKR. |

In a still further specific aspect, the antibody further comprises a human or murine constant region. In a still further aspect, the human constant region is selected from the group consisting of IgG1, IgG2, IgG2, IgG3, IgG4. In a still further specific aspect, the human constant region is IgG1. In a still further aspect, the murine constant region is selected from the group consisting of IgG1, IgG2A, IgG2B, IgG3. In a still further aspect, the murine constant region if IgG2A. In a still further specific aspect, the antibody has reduced or minimal effector function. In a still further specific aspect the minimal effector function results from an “effector-less Fc mutation” or aglycosylation. In still a further embodiment, the effector-less Fc mutation is an N297A or D265A/N297A substitution in the constant region.

In a still further embodiment, provided is an isolated anti-PDL1 antibody comprising a heavy chain and a light chain variable region sequence, wherein:

(a) the heavy chain sequence has at least 85% sequence identity to the heavy chain sequence: EVQLVESGGGLVQPGGSLRLSCAASGFTFSDSWIHWVRQAPGKGLEWVAWIS PYGGSTYYADSVKGRFTISADTSKNTAYLQMNSLRAEDTAVYYCARRHWPGGFDYWG QGTLVTVSA (SEQ ID NO:20), or

(b) the light chain sequence has at least 85% sequence identity to the light chain sequence: DIQMTQSPSSLSASVGDRVTITCRASQDVSTAVAWYQQKPGKAPKLLIY SASF LYSGVPSRFSGSGSGTDFTLTISSLQPEDFATYYCQQYLYHPATFGQGTKVEIKR (SEQ ID NO:21).

In a specific aspect, the sequence identity is 86%, 87%, 88%, 89%, 90%, 91%, 92%, 93%, 94%, 95%, 96%, 97%, 98%, 99% or 100%. In another aspect, the heavy chain variable region comprises one or more framework sequences juxtaposed between the HVRs as: (HC-FR1)-(HVR-H1)-(HC-FR2)-(HVR-H2)-(HC-FR3)-(HVR-H3)-(HC-FR4), and the light chain variable regions comprises one or more framework sequences juxtaposed between the HVRs as: (LC-FR1)-(HVR-L1)-(LC-FR2)-(HVR-L2)-(LC-FR3)-(HVR-L3)-(LC-FR4). In yet another aspect, the framework sequences are derived from human consensus framework sequences. In a further aspect, the heavy chain framework sequences are derived from a Kabat subgroup I, II, or III sequence. In a still further aspect, the heavy chain framework sequence is a VH subgroup III consensus framework. In a still further aspect, one or more of the heavy chain framework sequences is the following:

| (SEQ ID NO: 4) | |
|---|---|
| HC-FR1 | EVQLVESGGGLVQPGGSLRLSCAAS |
| (SEQ ID NO: 5) | |
| HC-FR2 | WVRQAPGKGLEWV |
| (SEQ ID NO: 6) | |
| HC-FR3 | RFTISADTSKNTAYLQMNSLRAEDTAVYYCAR |
| (SEQ ID NO: 7) | |
| HC-FR4 | WGQGTLVTVSA. |

In a still further aspect, the light chain framework sequences are derived from a Kabat kappa I, II, II or IV subgroup sequence. In a still further aspect, the light chain framework sequences are VL kappa I consensus framework. In a still further aspect, one or more of the light chain framework sequences is the following:

| (SEQ ID NO: 11) | |
|---|---|
| LC-FR1 | DIQMTQSPSSLSASVGDRVTITC |
| (SEQ ID NO: 12) | |
| LC-FR2 | WYQQKPGKAPKLLIY |
| (SEQ ID NO: 13) | |
| LC-FR3 | GVPSRFSGSGSGTDFTLTISSLQPEDFATYYC |
| (SEQ ID NO: 14) | |
| LC-FR4 | FGQGTKVEIKR. |

In a still further specific aspect, the antibody further comprises a human or murine constant region. In a still further aspect, the human constant region is selected from the group consisting of IgG1, IgG2, IgG2, IgG3, IgG4. In a still further specific aspect, the human constant region is IgG1. In a still further aspect, the murine constant region is selected from the group consisting of IgG1, IgG2A, IgG2B, IgG3. In a still further aspect, the murine constant region if IgG2A. In a still further specific aspect, the antibody has reduced or minimal effector function. In a still further specific aspect, the minimal effector function results from production in prokaryotic cells. In a still further specific aspect the minimal effector function results from an “effector-less Fc mutation” or aglycosylation. In still a further embodiment, the effector-less Fc mutation is an N297A or D265A/N297A substitution in the constant region.

In another further embodiment, provided is an isolated anti-PDL1 antibody comprising a heavy chain and a light chain variable region sequence, wherein:

(a) the heavy chain sequence has at least 85% sequence identity to the heavy chain sequence: EVQLVESGGGLVQPGGSLRLSCAASGFTFSDSWIHWVRQAPGKGLEWVAWIS PYGGSTYYADSVKGRFTISADTSKNTAYLQMNSLRAEDTAVYYCARRHWPGGFDYWG QGTLVTVSS (SEQ ID NO:24), or

(b) the light chain sequence has at least 85% sequence identity to the light chain sequence: DIQMTQSPSSLSASVGDRVTITCRASQDVSTAVAWYQQKPGKAPKLLIY SASF LYSGVPSRFSGSGSGTDFTLTISSLQPEDFATYYCQQYLYHPATFGQGTKVEIKR (SEQ ID NO:21).

In a still further embodiment, provided is an isolated anti-PDL1 antibody comprising a heavy chain and a light chain variable region sequence, wherein:

(a) the heavy chain sequence has at least 85% sequence identity to the heavy chain sequence: EVQLVESGGGLVQPGGSLRLSCAASGFTFSDSWIHWVRQAPGKGLEWVAWI SPYGGSTYYADSVKGRFTISADTSKNTAYLQMNSLRAEDTAVYYCARRHWPGGFDYW GQGTLVTVSSASTK (SEQ ID NO:28), or

(b) the light chain sequences has at least 85% sequence identity to the light chain sequence: DIQMTQSPSSLSASVGDRVTITCRASQDVSTAVAWYQQKPGKAPKLLIYSASF LYSGVPSRFSGSGSGTDFTLTISSLQPEDFATYYCQQYLYHPATFGQGTKVEIKR (SEQ ID NO:29).

In a specific aspect, the sequence identity is 86%, 87%, 88%, 89%, 90%, 91%, 92%, 93%, 94%, 95%, 96%, 97%, 98%, 99% or 100%. In another aspect, the heavy chain variable region comprises one or more framework sequences juxtaposed between the HVRs as: (HC-FR1)-(HVR-H1)-(HC-FR2)-(HVR-H2)-(HC-FR3)-(HVR-H3)-(HC-FR4), and the light chain variable regions comprises one or more framework sequences juxtaposed between the HVRs as: (LC-FR1)-(HVR-L1)-(LC-FR2)-(HVR-L2)-(LC-FR3)-(HVR-L3)-(LC-FR4). In yet another aspect, the framework sequences are derived from human consensus framework sequences. In a further aspect, the heavy chain framework sequences are derived from a Kabat subgroup I, II, or III sequence. In a still further aspect, the heavy chain framework sequence is a VH subgroup III consensus framework. In a still further aspect, one or more of the heavy chain framework sequences is the following:

| (SEQ ID NO: 4) | |
|---|---|
| HC-FR1 | EVQLVESGGGLVQPGGSLRLSCAAS |
| (SEQ ID NO: 5) | |
| HC-FR2 | WVRQAPGKGLEWV |
| (SEQ ID NO: 6) | |
| HC-FR3 | RFTISADTSKNTAYLQMNSLRAEDTAVYYCAR |
| (SEQ ID NO: 25) | |
| HC-FR4 | WGQGTLVTVSS. |

In a still further aspect, the light chain framework sequences are derived from a Kabat kappa I, II, II or IV subgroup sequence. In a still further aspect, the light chain framework sequences are VL kappa I consensus framework. In a still further aspect, one or more of the light chain framework sequences is the following:

| (SEQ ID NO: 11) | |
|---|---|
| LC-FR1 | DIQMTQSPSSLSASVGDRVTITC |
| (SEQ ID NO: 12) | |
| LC-FR2 | WYQQKPGKAPKLLIY |
| (SEQ ID NO: 13) | |
| LC-FR3 | GVPSRFSGSGSGTDFTLTISSLQPEDFATYYC |
| (SEQ ID NO: 14) | |
| LC-FR4 | FGQGTKVEIKR. |

In a still further specific aspect, the antibody further comprises a human or murine constant region. In a still further aspect, the human constant region is selected from the group consisting of IgG1, IgG2, IgG2, IgG3, IgG4. In a still further specific aspect, the human constant region is IgG1. In a still further aspect, the murine constant region is selected from the group consisting of IgG1, IgG2A, IgG2B, IgG3. In a still further aspect, the murine constant region if IgG2A. In a still further specific aspect, the antibody has reduced or minimal effector function. In a still further specific aspect, the minimal effector function results from production in prokaryotic cells. In a still further specific aspect the minimal effector function results from an “effector-less Fc mutation” or aglycosylation. In still a further embodiment, the effector-less Fc mutation is an N297A or D265A/N297A substitution in the constant region.

In yet another embodiment, the anti-PDL1 antibody is MPDL3280A (CAS Registry Number: 1422185-06-5). In a still further embodiment, provided is an isolated anti-PDL1 antibody comprising a heavy chain variable region comprising the heavy chain variable region amino acid sequence from SEQ ID NO:24 or SEQ ID NO:28 and/or a light chain variable region comprising the light chain variable region amino acid sequence from SEQ ID NO:21. In a still further embodiment, provided is an isolated anti-PDL1 antibody comprising a heavy chain and/or a light chain sequence, wherein:

(a) the heavy chain sequence has at least 85%, at least 90%, at least 91%, at least 92%, at least 93%, at least 94%, at least 95%, at least 96%, at least 97%, at least 98%, at least 99% or 100% sequence identity to the heavy chain sequence: EVQLVESGGGLVQPGGSLRLSCAASGFTFSDSWIHWVRQAPGKGLEWVAWISPYGGST YYADSVKGRFTISADTSKNTAYLQMNSLRAEDTAVYYCARRHWPGGFDYWGQGTLVT VSSASTKGPSVFPLAPSSKSTSGGTAALGCLVKDYFPEPVTVSWNSGALTSGVHTFPAVL QSSGLYSLSSVVTVPSSSLGTQTYICNVNHKPSNTKVDKKVEPKSCDKTHTCPPCPAPEL LGGPSVFLFPPKPKDTLMISRTPEVTCVVVDVSHEDPEVKFNWYVDGVEVHNAKTKPRE EQYASTYRVVSVLTVLHQDWLNGKEYKCKVSNKALPAPIEKTISKAKGQPREPQVYTLP PSREEMTKNQVSLTCLVKGFYPSDIAVEWESNGQPENNYKTTPPVLDSDGSFFLYSKLT VDKSRWQQGNVFSCSVMHEALHNHYTQKSLSLSPG (SEQ ID NO:26), or

(b) the light chain sequences has at least 85%, at least 90%, at least 91%, at least 92%, at least 93%, at least 94%, at least 95%, at least 96%, at least 97%, at least 98%, at least 99% or 100% sequence identity to the light chain sequence:

(SEQ ID NO: 27)
DIQMTQSPSSLSASVGDRVTITCRASQDVSTAVAWYQQKPGKAPKLLIYS
ASFLYSGVPSRFSGSGSGTDFTLTISSLQPEDFATYYCQQYLYHPATFGQ
GTKVEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC.

In a still further embodiment, the invention provides for compositions comprising any of the above described anti-PDL1 antibodies in combination with at least one pharmaceutically-acceptable carrier.

In a still further embodiment, provided is an isolated nucleic acid encoding a light chain or a heavy chain variable region sequence of an anti-PDL1 antibody, wherein:
(a) the heavy chain further comprises and HVR-H1, HVR-H2 and an HVR-H3 sequence having at least 85% sequence identity to GFTFSDSWIH (SEQ ID NO:15), AWISPYGGSTYYADSVKG (SEQ ID NO:16) and RHWPGGFDY (SEQ ID NO:3), respectively, and
(b) the light chain further comprises an HVR-L1, HVR-L2 and an HVR-L3 sequence having at least 85% sequence identity to RASQDVSTAVA (SEQ ID NO:17), SASFLYS (SEQ ID NO:18) and QQYLYHPAT (SEQ ID NO:19), respectively.

In a specific aspect, the sequence identity is 86%, 87%, 88%, 89%, 90%, 91%, 92%, 93%, 94%, 95%, 96%, 97%, 98%, 99% or 100%. In aspect, the heavy chain variable region comprises one or more framework sequences juxtaposed between the HVRs as: (HC-FR1)-(HVR-H1)-(HC-FR2)-(HVR-H2)-(HC-FR3)-(HVR-H3)-(HC-FR4), and the light chain variable regions comprises one or more framework sequences juxtaposed between the HVRs as: (LC-FR1)-(HVR-L1)-(LC-FR2)-(HVR-L2)-(LC-FR3)-(HVR-L3)-(LC-FR4). In yet another aspect, the framework sequences are derived from human consensus framework sequences. In a further aspect, the heavy chain framework sequences are derived from a Kabat subgroup I, II, or III sequence. In a still further aspect, the heavy chain framework sequence is a VH subgroup III consensus framework. In a still further aspect, one or more of the heavy chain framework sequences is the following:

(SEQ ID NO: 4)
HC-FR1 EVQLVESGGGLVQPGGSLRLSCAAS
(SEQ ID NO: 5)
HC-FR2 WVRQAPGKGLEWV
(SEQ ID NO: 6)
HC-FR3 RFTISADTSKNTAYLQMNSLRAEDTAVYYCAR
(SEQ ID NO: 7)
HC-FR4 WGQGTLVTVSA.

In a still further aspect, the light chain framework sequences are derived from a Kabat kappa I, II, II or IV subgroup sequence. In a still further aspect, the light chain framework sequences are VL kappa I consensus framework. In a still further aspect, one or more of the light chain framework sequences is the following:

(SEQ ID NO: 11)
LC-FR1 DIQMTQSPSSLSASVGDRVTITC
(SEQ ID NO: 12)
LC-FR2 WYQQKPGKAPKLLIY
(SEQ ID NO: 13)
LC-FR3 GVPSRFSGSGSGTDFTLTISSLQPEDFATYYC
(SEQ ID NO: 14)
LC-FR4 FGQGTKVEIKR.

In a still further specific aspect, the antibody described herein (such as an anti-PD-1 antibody, an anti-PDL1 antibody, or an anti-PDL2 antibody) further comprises a human or murine constant region. In a still further aspect, the human constant region is selected from the group consisting of IgG1, IgG2, IgG2, IgG3, IgG4. In a still further specific aspect, the human constant region is IgG1. In a still further aspect, the murine constant region is selected from the group consisting of IgG1, IgG2A, IgG2B, IgG3. In a still further aspect, the murine constant region if IgG2A. In a still further specific aspect, the antibody has reduced or minimal effector function. In a still further specific aspect, the minimal effector function results from production in prokaryotic cells. In a still further specific aspect the minimal effector function results from an “effector-less Fc mutation” or aglycosylation. In still a further aspect, the effector-less Fc mutation is an N297A or D265A/N297A substitution in the constant region.

In a still further aspect, provided herein are nucleic acids encoding any of the antibodies described herein. In some embodiments, the nucleic acid further comprises a vector suitable for expression of the nucleic acid encoding any of the previously described anti-PDL1, anti-PD-1, or anti-PDL2 antibodies. In a still further specific aspect, the vector further comprises a host cell suitable for expression of the nucleic acid. In a still further specific aspect, the host cell is a eukaryotic cell or a prokaryotic cell. In a still further specific aspect, the eukaryotic cell is a mammalian cell, such as Chinese Hamster Ovary (CHO).

The antibody or antigen binding fragment thereof, may be made using methods known in the art, for example, by a process comprising culturing a host cell containing nucleic acid encoding any of the previously described anti-PDL1, anti-PD-1, or anti-PDL2 antibodies or antigen-binding fragment in a form suitable for expression, under conditions suitable to produce such antibody or fragment, and recovering the antibody or fragment.

In some embodiments, the isolated anti-PDL1 antibody is aglycosylated. Glycosylation of antibodies is typically either N-linked or O-linked. N-linked refers to the attachment of the carbohydrate moiety to the side chain of an asparagine residue. The tripeptide sequences asparagine-X-serine and asparagine-X-threonine, where X is any amino acid except proline, are the recognition sequences for enzymatic attachment of the carbohydrate moiety to the asparagine side chain. Thus, the presence of either of these tripeptide sequences in a polypeptide creates a potential glycosylation site. O-linked glycosylation refers to the attachment of one of the sugars N-aceylgalactosamine, galactose, or xylose to a hydroxyamino acid, most commonly serine or threonine, although 5-hydroxyproline or 5-hydroxylysine may also be used. Removal of glycosylation sites form an antibody is conveniently accomplished by altering the amino acid sequence such that one of the above-described tripeptide sequences (for N-linked glycosylation sites) is removed. The alteration may be made by substitution of an asparagine, serine or threonine residue within the glycosylation site another amino acid residue (e.g., glycine, alanine or a conservative substitution).

In any of the embodiments herein, the isolated anti-PDL1 antibody can bind to a human PDL1, for example a human PDL1 as shown in UniProtKB/Swiss-Prot Accession No. Q9NZQ7.1, or a variant thereof.

In a still further embodiment, the invention provides for a composition comprising an anti-PDL1, an anti-PD-1, or an anti-PDL2 antibody or antigen binding fragment thereof as provided herein and at least one pharmaceutically acceptable carrier. In some embodiments, the anti-PDL1, anti-PD-1, or anti-PDL2 antibody or antigen binding fragment thereof administered to the individual is a composition comprising one or more pharmaceutically acceptable carrier. Any of the pharmaceutically acceptable carriers described herein or known in the art may be used.

In some embodiments, the anti-PDL1 antibody described herein is in a formulation comprising the antibody at an amount of about 60 mg/mL, histidine acetate in a concentration of about 20 mM, sucrose in a concentration of about 120 mM, and polysorbate (e.g., polysorbate 20) in a concentration of 0.04% (w/v), and the formulation has a pH of about 5.8. In some embodiments, the anti-PDL1 antibody described herein is in a formulation comprising the antibody in an amount of about 125 mg/mL, histidine acetate in a concentration of about 20 mM, sucrose is in a concentration of about 240 mM, and polysorbate (e.g., polysorbate 20) in a concentration of 0.02% (w/v), and the formulation has a pH of about 5.5.

### IV. IL-17 Binding Antagonists

Provided herein are methods for treating or delaying progression of cancer in an individual comprising administering to the individual an effective amount of a PD-1 axis binding antagonist and an IL-17 binding antagonist.

In some embodiments, an IL-17 binding antagonist inhibits the binding of IL-17 to the IL-17 receptor. It has been demonstrated that IL-17 activity is mediated through binding to its unique cell surface receptor, IL-1.7R (see US Application Publication No, 20100055103 for more detailed description). Additional descriptions of IL-17 and IL-17R may be found in Gaffen, Nat. Rev. Immunol., 9:556-67 (2009).

As described herein, the term “IL-17” encompasses one or more members of the IL-17 family of cytokines, such as IL-17A and IL-17F (inter alia), as well as a single IL-17 family cytokine polypeptide or a dimer of IL-17 family cytokine monomers (e.g., IL-17AA, IL-17FF, or IL-17AF). In addition, the term “IL-17” further encompasses “full-length” and unprocessed IL-17 as well as any form of IL-17 that results from processing in the cell (e.g., mature protein). The term also encompasses naturally occurring variants and isoforms of IL-17, e.g., splice variants or allelic variants. Descriptions of exemplary IL-17 family members and sequences are provided at www.uniprot.org/uniprot/Q16552 and www.uniprot.org/uniprot/Q96PD4.

In some embodiments, an IL-17 binding antagonist inhibits the binding of an IL-17A homodimer to an IL-17 receptor. In some embodiments, an IL-17 binding antagonist inhibits the binding of an IL-17F homodimer to an IL-17 receptor. In some embodiments, an IL-17 binding antagonist inhibits the binding of an IL-17A/IL-17F heterodimer to an IL-17 receptor.

In some embodiments, the IL-17 binding antagonist is an antibody (e.g., a human antibody, a humanized antibody, or a chimeric antibody). In some embodiments, the IL-17 binding antagonist is a monoclonal antibody. In some embodiments, the IL-17 binding antagonist is an antibody fragment selected from the group consisting of Fab, Fab′-SH, Fv, scFv, and (Fab′)₂ fragments. In some embodiments, the IL-17 binding antagonist is a humanized antibody or a human antibody.

In some embodiments, the IL-17 binding antagonist is an anti-IL-17 antibody. A number of anti-IL-17 antibodies are known in the art. Several exemplary anti-IL-17 antibodies, sequences, and references describing anti-IL-17 antibodies in further detail are provided below. In some embodiments, an anti-IL-17 antibody binds one or more of an IL-17A homodimer, an IL-17F homodimer, and an IL-17A/IL-17F heterodimer.

In some embodiments, the anti-IL-17 antibody is an anti-IL-17 antibody described in U.S. Pat. No. 8,771,697. For example, in some embodiments, the anti-IL-17 antibody is 30D12BF, or a variant thereof, as described in U.S. Pat. No. 8,771,697. In some embodiments, an IL-17 antibody comprises one, two, three, four, five, or fix CDRs of antibody 30D12BF, as described in U.S. Pat. No. 8,771,697. In some embodiments, an IL-17 antibody comprises a heavy chain variable region and/or a light chain variable region of antibody 30D12BF, as described in U.S. Pat. No. 8,771,697. In some embodiments, In some embodiments, the anti-IL-17 antibody comprises a heavy chain variable region (VH region) having at least 85% sequence identity to the amino acid sequence: QVQLVQSGAEVKKPGASVKVSCKASGYTFTSYDINWVRQATGQGLEWMGWLNPDSG VIRYAQKFQGRVTMTRDTSISTAYMELSSLRSEDTAVYYCAREWFGELPSYYFYSGMD VWGQGTTVTVSS (SEQ ID NO:30), and/or a light chain variable region (VL region) having at least 85% sequence identity to the amino acid sequence:

(SEQ ID NO: 31)
EIVLTQSPATLSLSPGERATLSCRASQSVSSYLAWYQQKPGQAPRLLIY
DASNRATGIPARFSGSGSGTDFTLTISSLEPEDFAVYYCQQRSNWPPTF
GPGTKVDIK.

In a specific aspect, the sequence identity is 86%, 87%, 88%, 89%, 90%, 91%, 92%, 93%, 94%, 95%, 96%, 97%, 98%, 99% or 100%.

In one embodiment, the anti-IL-17 antibody contains a heavy chain variable region polypeptide comprising a CDR-H1, CDR-H2 and CDR-H3 sequence, wherein:

(SEQ ID NO: 32)
(a) the CDR-H1 sequence is SYDIN;
(SEQ ID NO: 33)
(b) the CDR-H2 sequence is WLNPDSGVIRYAQKFQG;
and
(SEQ ID NO: 34)
(c) the CDR-H3 sequence is EWFGELPSYYFYSGMDV.

In one embodiment, the anti-IL-17 antibody contains a light chain variable region polypeptide comprising a CDR-L1, CDR-L2 and CDR-L3 sequence, wherein:

(SEQ ID NO: 35)
(a) the CDR-L1 sequence is RASQSVSSYLA;
(SEQ ID NO: 36)
(b) the CDR-L2 sequence is DASNRAT;
and
(SEQ ID NO: 37)
(c) the CDR-L3 sequence is QQRSNWPPT.

In some embodiments, the anti-IL-17 antibody is 29D8, or a variant thereof, as described in U.S. Pat. No. 8,771,697. In some embodiments, an IL-17 antibody comprises one, two, three, four, five, or fix CDRs of antibody 29D8, as described in U.S. Pat. No. 8,771,697. In some embodiments, an IL-17 antibody comprises a heavy chain variable region and/or a light chain variable region of antibody 29D8, as described in U.S. Pat. No. 8,771,697. In some embodiments, the anti-IL-17 antibody comprises a heavy chain variable region (VH region) having at least 85% sequence identity to the amino acid sequence: QVQLVQSGAEVKKPGASVKVSCKAFAYTFSTYGISWVRQAPGQGLEWMGWISAYNSN TNYAQKVQGRITMTTDTSTRTAYMELRGLRSDDTAVYFCATFFGGHSGYHYGLDVWG QGTTVTVSS (SEQ ID NO:38), and/or a light chain variable region (VL region) having at least 85% sequence identity to the amino acid sequence:

(SEQ ID NO: 39)
EIVLXQSPATLSLSPGERATLSCRASQSVSSYLAWYQQKPGQAPRLLXY
DASNRATGIPARFSGSGSGTDFTLTISSLEPEDFAVYYCQQRSNWPPYT
FGQGTKLEIK.

In a specific aspect, the sequence identity is 86%, 87%, 88%, 89%, 90%, 91%, 92%, 93%, 94%, 95%, 96%, 97%, 98%, 99% or 100%.

In one embodiment, the anti-IL-17 antibody contains a heavy chain variable region polypeptide comprising a CDR-H1, CDR-H2 and CDR-H3 sequence, wherein:

(SEQ ID NO: 40)
(a) the CDR-H1 sequence is TYGIS;
(SEQ ID NO: 41)
(b) the CDR-H2 sequence is WISAYNSNTNYAQKVQG;
and
(SEQ ID NO: 42)
(c) the CDR-H3 sequence is FFGGHSGYHYGLDV.

In one embodiment, the anti-IL-17 antibody contains a light chain variable region polypeptide comprising a CDR-L1, CDR-L2 and CDR-L3 sequence, wherein:

(SEQ ID NO: 43)
(a) the CDR-L1 sequence is RASQSVSSYLA;
(SEQ ID NO: 44)
(b) the CDR-L2 sequence is DASNRAT;
and
(SEQ ID NO: 45)
(c) the CDR-L3 sequence is QQRSNWPPYT.

In some embodiments, the anti-IL-17 antibody is 15E6FK, or a variant thereof, as described in U.S. Pat. No. 8,771,697. In some embodiments, an IL-17 antibody comprises one, two, three, four, five, or fix CDRs of antibody 15E6FK, as described in U.S. Pat. No. 8,771,697. In some embodiments, an IL-17 antibody comprises a heavy chain variable region and/or a light chain variable region of antibody 15E6FK, as described in U.S. Pat. No. 8,771,697. In some embodiments, the anti-IL-17 antibody comprises a heavy chain variable region (VH region) having at least 85% sequence identity to the amino acid sequence: EVQLVESGGGLVQPGRSLRLSCAASGFTFDDYAMHWVRQAPGKGLEWVSGINWSSGGI GYADSVKGRFTISRDNAKNSLYLQMNSLRAEDTALYYCARDIGGFGEFYWNFGLWGR GTLVTVSS (SEQ ID NO:46), and/or a light chain variable region (VL region) having at least 85% sequence identity to the amino acid sequence:

(SEQ ID NO: 47)
EIVLTQSPATLSLSPGERATLSCRASQSVRSYLAWYQQKPGQAPRLLI
YDASNRATGIPARFSGSGSGTDFTLTISSLEPEDFAVYYCQQRSNWPP
ATFGGGTKVEIK.

In a specific aspect, the sequence identity is 86%, 87%, 88%, 89%, 90%, 91%, 92%, 93%, 94%, 95%, 96%, 97%, 98%, 99% or 100%.

In one embodiment, the anti-IL-17 antibody contains a heavy chain variable region polypeptide comprising a CDR-H1, CDR-H2 and CDR-H3 sequence, wherein:

(SEQ ID NO: 48)
(a) the CDR-H1 sequence is DYAMH;
(SEQ ID NO: 49)
(b) the CDR-H2 sequence is GINWSSGGIGYADSVKG;
and
(SEQ ID NO: 50)
(c) the CDR-H3 sequence is DIGGFGEFYWNFGL.

In one embodiment, the anti-IL-17 antibody contains a light chain variable region polypeptide comprising a CDR-L1, CDR-L2 and CDR-L3 sequence, wherein:

(SEQ ID NO: 51)
(a) the CDR-L1 sequence is RASQSVRSYLA;
(SEQ ID NO: 52)
(b) the CDR-L2 sequence is DASNRAT;
and
(SEQ ID NO: 53)
(c) the CDR-L3 sequence is QQRSNWPPAT.

In some embodiments, the anti-IL-17 antibody is 39F12A, or a variant thereof, as described in U.S. Pat. No. 8,771,697. In some embodiments, an IL-17 antibody comprises one, two, three, four, five, or fix CDRs of antibody 39F12A, as described in U.S. Pat. No. 8,771,697. In some embodiments, an IL-17 antibody comprises a heavy chain variable region and/or a light chain variable region of antibody 39F12A, as described in U.S. Pat. No. 8,771,697. In some embodiments, the anti-IL-17 antibody comprises a heavy chain variable region (VH region) having at least 85% sequence identity to the amino acid sequence: QVQLVQSGAEVKKPGSSVKVSCKASGGTLSSYAFSWVRQAPGQGLEWMGGIIPFFGTT NYAQKFQGRVIITADESTNTAYMELSGLRSEDTAVYYCARDRDYYGLGSPFYYYGMDV WGQGTTVTVSS (SEQ ID NO:54), and/or a light chain variable region (VL region) having at least 85% sequence identity to the amino acid sequence:

(SEQ ID NO: 55)
EIVLTQSPDFQSVTPKEKVTITCRASQSIGSSLHWYQQKPDQSPKLLIKY
ASQSFSGVPSRFSGSGSGTDFTLTINSLEAEDAATYYCHQSSSLPWTFGQ
GTKVEIK.

In a specific aspect, the sequence identity is 86%, 87%, 88%, 89%, 90%, 91%, 92%, 93%, 94%, 95%, 96%, 97%, 98%, 99% or 100%.

In one embodiment, the anti-IL-17 antibody contains a heavy chain variable region polypeptide comprising a CDR-H1, CDR-H2 and CDR-H3 sequence, wherein:

(SEQ ID NO: 56)
(a) the CDR-H1 sequence is SYAFS;
(SEQ ID NO: 57)
(b) the CDR-H2 sequence is GIIPFFGTTNYAQKFQG;
and
(SEQ ID NO: 58)
(c) the CDR-H3 sequence is DRDYYGLGSPFYYYGMDV.

In one embodiment, the anti-IL-17 antibody contains a light chain variable region polypeptide comprising a CDR-L1, CDR-L2 and CDR-L3 sequence, wherein:

(SEQ ID NO: 59)
(a) the CDR-L1 sequence is RASQSIGSSLH;
(SEQ ID NO: 60)
(b) the CDR-L2 sequence is YASQSFS;
and
(SEQ ID NO: 61)
(c) the CDR-L3 sequence is HQSSSLPWT.

In some embodiments, the anti-IL-17 antibody specifically binds to IL-17A. In some embodiments, the anti-IL-17 antibody specifically binds to IL-17F.

In some embodiments, the anti-IL-17 antibody is an anti-IL-17 antibody (e.g., an antibody that binds to IL-17AA and IL-17AF) as described in U.S. Pat. No. 7,807,155. In one embodiment, the anti-IL-17 antibody is secukinumab.

In some embodiments, the anti-IL-17 antibody is an anti-IL-17 antibody (e.g., an antibody that binds to IL-17AA and IL-17AF) as described in U.S. Pat. No. 7,838,638. In one embodiment, the anti-IL-17 antibody is ixekizumab.

IL-17A/F (i.e., a heterodimeric IL-17 including IL-17A and IL-17F monomers) has been described to as a target for treating various immune-mediated diseases (Chang and Dong, Cell Res. 17(5):435-40 (200′7)); an IL-17A/F protein produced by mouse Th17 cells has been shown to induce airway neutrophil recruitment and thus having an in vivo function in airway neutrophilia (Liang et al., J immunol 179(11):7791-9 (2007)); and the human IL-17A/F heterodimeric cytokine has been reported to signal through the IL-17RA/IL-17RC receptor complex (Wright et al., J Immunol 181(4):2799-805 (2008)).

In some embodiments, the anti-IL-17 antibody specifically binds to IL-17A and IL-17F. Such an antibody is known in the art as a cross-reactive antibody. A “cross-reactive antibody” may refer to an antibody which recognizes identical or similar epitopes on more than one antigen. Thus, the cross-reactive antibodies of the present disclosure may recognize identical or similar epitopes present on both IL-17A and IL-17F. In a particular embodiment, the cross-reactive antibody uses the same or essentially the same paratope to bind to both IL-17A and IL-17F (as used herein, the term “paratope” may refer to the part of an antibody that hinds to a target antigen). Preferably, the cross-reactive antibodies herein also block both IL-17A and IL-17F function (activity). Further description of properties of cross-reactive IL-17 antibodies, and exemplary methods for generating cross-reactive IL-17 antibodies may be found, e.g., in U.S. Patent Publication No. US20100055103, which is incorporated herein by reference.

In some embodiments, the anti-IL-17 antibody is an anti-IL-17 antibody (e.g., a cross-reactive antibody) as described in PCT Publication No. WO2007106769, which is incorporated herein by reference.

In some embodiments, the anti-IL-17 antibody is an anti-IL-17 antibody (e.g., a cross-reactive antibody) as described in PCT Publication No. WO2012095662, which is incorporated herein by reference. In one embodiment, the anti-IL-17 antibody is bimekizumab.

In some embodiments, the IL-17 binding antagonist is an anti-IL-17 receptor antibody. In some embodiments, the anti-IL-17 receptor antibody specifically binds an IL-17 receptor that interacts with IL-17A and/or IL-17F (e.g., an IL-17A homodimer, an IL-17F homodimer, or an IL-17A/IL-17F heterodimer). In some embodiments, an anti-IL-17 receptor antibody binds an epitope on an extracellular domain of an IL-17 receptor. Descriptions of exemplary IL-17 receptors and sequences are provided at www.uniprot.org/uniprot/Q96F46 and www.uniprot.org/uniprot/Q8NAC3.

In some contexts, heterodimeric complexes of the IL-17 receptor are known to mediate IL-17 signaling (see, e.g., Wright et al., J Immunol 181(40:2799-805 (2008)). In some embodiments, the anti-IL-17 receptor antibody specifically binds an IL-17 receptor complex, such as the IL-17RA/IL-17RC receptor complex or the IL-17RA/IL-17RB receptor complex.

Anti-IL-17 receptor antibodies are known in the art. For example, in some embodiments, the anti-IL-17 receptor antibody as described in U.S. Pat. No. 7,767,206. In one embodiment, the anti-IL-17 receptor antibody is brodalumab.

In some embodiments, the IL-17 binding antagonist is a soluble polypeptide comprising at least one exon from an IL-17 receptor. Such a polypeptide may interact with an IL-17 family cytokine and inhibit its ability to bind an endogenous IL-17 receptor. In some embodiments, the soluble polypeptide comprising at least one exon from an IL-17 receptor includes a portion of an extracellular domain from the IL-17 receptor. In some embodiments, the soluble polypeptide comprises at least one exon from IL-17RA and at least one exon from IL-17RC. In some embodiments, the soluble polypeptide comprising at least one exon from an IL-17 receptor is a soluble polypeptide as described in PCT Publication No. WO2007038703.

In some embodiments, the isolated anti-IL-17 and/or anti-IL-17 receptor antibody is aglycosylated. Glycosylation of antibodies is typically either N-linked or O-linked. N-linked refers to the attachment of the carbohydrate moiety to the side chain of an asparagine residue. The tripeptide sequences asparagine-X-serine and asparagine-X-threonine, where X is any amino acid except proline, are the recognition sequences for enzymatic attachment of the carbohydrate moiety to the asparagine side chain. Thus, the presence of either of these tripeptide sequences in a polypeptide creates a potential glycosylation site. O-linked glycosylation refers to the attachment of one of the sugars N-aceylgalactosamine, galactose, or xylose to a hydroxyamino acid, most commonly serine or threonine, although 5-hydroxyproline or 5-hydroxylysine may also be used. Removal of glycosylation sites form an antibody is conveniently accomplished by altering the amino acid sequence such that one of the above-described tripeptide sequences (for N-linked glycosylation sites) is removed. The alteration may be made by substitution of an asparagine, serine or threonine residue within the glycosylation site another amino acid residue (e.g., glycine, alanine or a conservative substitution).

In some embodiments, an anti-IL-17 antibody or antigen binding fragment thereof, anti-IL-17 receptor antibody or antigen binding fragment thereof, and/or a soluble polypeptide comprising at least one exon from an IL-17 receptor as provided herein is administered to the individual in a composition comprising one or more pharmaceutically acceptable carrier. Any of the pharmaceutically acceptable carriers described herein or known in the art may be used.

### V. Antibody Preparation

As described above, in some embodiments, the PD-1 binding antagonist is an antibody (e.g., an anti-PD-1 antibody, an anti-PDL1 antibody, or an anti-PDL2 antibody). In some embodiments, the IL-17 binding antagonist is an antibody (e.g., an anti-IL-17 antibody, or an anti-IL-17 receptor antibody). The antibodies described herein may be prepared using techniques available in the art for generating antibodies, exemplary methods of which are described in more detail in the following sections.

The antibody is directed against an antigen of interest. For example, the antibody may be directed against PD-1 (such as human PD-1), PDL1 (such as human PDL1), PDL2 (such as human PDL2), an IL-17 (such as IL-17A and/or IL-17F, including human IL-17A and/or human IL-17F), or an IL-17 receptor (such as IL-17RA and/or IL-17RC, including human IL-17RA and/or human IL-17RC). Preferably, the antigen is a biologically important polypeptide and administration of the antibody to a mammal suffering from a disorder can result in a therapeutic benefit in that mammal.

In certain embodiments, an antibody described herein has a dissociation constant (Kd) of ≦1 μM, ≦150 nM, ≦100 nM, ≦50 nM, ≦10 nM, ≦1 nM, ≦0.1 nM, ≦0.01 nM, or ≦0.001 nM (e.g. 10⁻⁸M or less, e.g. from 10⁻⁸M to 10⁻¹³M, e.g., from 10⁻⁹M to 10⁻¹³ M).

In one embodiment, Kd is measured by a radiolabeled antigen binding assay (RIA) performed with the Fab version of an antibody of interest and its antigen as described by the following assay. Solution binding affinity of Fabs for antigen is measured by equilibrating Fab with a minimal concentration of (¹²⁵I)-labeled antigen in the presence of a titration series of unlabeled antigen, then capturing bound antigen with an anti-Fab antibody-coated plate (see, e.g., Chen et al., J. Mol. Biol. 293:865-881(1999)). To establish conditions for the assay, MICROTITER® multi-well plates (Thermo Scientific) are coated overnight with 5 μg/ml of a capturing anti-Fab antibody (Cappel Labs) in 50 mM sodium carbonate (pH 9.6), and subsequently blocked with 2% (w/v) bovine serum albumin in PBS for two to five hours at room temperature (approximately 23° C.). In a non-adsorbent plate (Nunc #269620), 100 pM or 26 pM [¹²⁵I]-antigen are mixed with serial dilutions of a Fab of interest. The Fab of interest is then incubated overnight; however, the incubation may continue for a longer period (e.g., about 65 hours) to ensure that equilibrium is reached. Thereafter, the mixtures are transferred to the capture plate for incubation at room temperature (e.g., for one hour). The solution is then removed and the plate washed eight times with 0.1% polysorbate 20 (TWEEN-20®) in PBS. When the plates have dried, 150 μl/well of scintillant (MICROSCINT-20™; Packard) is added, and the plates are counted on a TOPCOUNT™ gamma counter (Packard) for ten minutes. Concentrations of each Fab that give less than or equal to 20% of maximal binding are chosen for use in competitive binding assays.

According to another embodiment, Kd is measured using surface plasmon resonance assays using a BIACORE®-2000 or a BIACORE®-3000 (BIAcore, Inc., Piscataway, N.J.) at 25° C. with immobilized antigen CM5 chips at ˜10 response units (RU). Briefly, carboxymethylated dextran biosensor chips (CM5, BIACORE, Inc.) are activated with N-ethyl-N′-(3-dimethylaminopropyl)-carbodiimide hydrochloride (EDC) and N-hydroxysuccinimide (NHS) according to the supplier's instructions. Antigen is diluted with 10 mM sodium acetate, pH 4.8, to 5 μg/ml (˜0.2 μM) before injection at a flow rate of 5 μl/minute to achieve approximately 10 response units (RU) of coupled protein. Following the injection of antigen, 1 M ethanolamine is injected to block unreacted groups. For kinetics measurements, two-fold serial dilutions of Fab (0.78 nM to 500 nM) are injected in PBS with 0.05% polysorbate 20 (TWEEN-20™) surfactant (PBST) at 25° C. at a flow rate of approximately 25 μl/min Association rates (kₒₙ) and dissociation rates (k_{off}) are calculated using a simple one-to-one Langmuir binding model (BIACORE® Evaluation Software version 3.2) by simultaneously fitting the association and dissociation sensorgrams. The equilibrium dissociation constant (Kd) is calculated as the ratio k_{off}/kₒₙ. See, e.g., Chen et al., J. Mol. Biol. 293:865-881 (1999). If the on-rate exceeds 10⁶ M⁻¹ s⁻¹ by the surface plasmon resonance assay above, then the on-rate can be determined by using a fluorescent quenching technique that measures the increase or decrease in fluorescence emission intensity (excitation=295 nm; emission=340 nm, 16 nm band-pass) at 25° C. of a 20 nM anti-antigen antibody (Fab form) in PBS, pH 7.2, in the presence of increasing concentrations of antigen as measured in a spectrometer, such as a stop-flow equipped spectrophometer (Aviv Instruments) or a 8000-series SLM-AMINCO™ spectrophotometer (ThermoSpectronic) with a stirred cuvette.

In some embodiments, an anti-IL-17 antibody as described herein exhibits a binding affinity of at least 100 pM or less against human IL-17A homodimer, a binding affinity of at least 300 pM or less against human IL-17F homodimer, a binding affinity of at least 400 pM or less against human IL-17A/IL-17F heterodimeric complex, a neutralizing ability of at least 40 nM or less against the human IL-17A homodimer, a neutralizing ability of at least 120 nM or less against the human IL-17F homodimer, and a neutralizing ability of at least 31 nM or less against the human IL-17A/IL-17F heterodimeric complex. In these embodiments, binding affinity may be measured by surface plasmon resonance as described in U.S. Pat. No. 8,771,697, and neutralizing ability may be determined by measuring IL-6 secretion by the human IL-17A homodimer, the human IL-17F homodimer or the human IL-17A/IL-17F heterodimeric complex-stimulated mouse or human embryonic fibroblasts as described in U.S. Pat. No. 8,771,697.

Antibody Fragments

In certain embodiments, an antibody described herein is an antibody fragment. Antibody fragments include, but are not limited to, Fab, Fab′, Fab′-SH, F(ab′)₂, Fv, and scFv fragments, and other fragments described below. For a review of certain antibody fragments, see Hudson et al. Nat. Med. 9:129-134 (2003). For a review of scFv fragments, see, e.g., Pluckthün, in The Pharmacology of Monoclonal Antibodies, vol. 113, Rosenburg and Moore eds., (Springer-Verlag, New York), pp. 269-315 (1994); see also WO 93/16185; and U.S. Pat. Nos. 5,571,894 and 5,587,458. For discussion of Fab and F(ab′)₂ fragments comprising salvage receptor binding epitope residues and having increased in vivo half-life, see U.S. Pat. No. 5,869,046.

Diabodies are antibody fragments with two antigen-binding sites that may be bivalent or bispecific. See, for example, EP 404,097; WO 1993/01161; Hudson et al., Nat. Med. 9:129-134 (2003); and Hollinger et al., Proc. Natl. Acad. Sci. USA 90: 6444-6448 (1993). Triabodies and tetrabodies are also described in Hudson et al., Nat. Med. 9:129-134 (2003).

Single-domain antibodies are antibody fragments comprising all or a portion of the heavy chain variable domain or all or a portion of the light chain variable domain of an antibody. In certain embodiments, a single-domain antibody is a human single-domain antibody (Domantis, Inc., Waltham, Mass.; see, e.g., U.S. Pat. No. 6,248,516 B1).

Antibody fragments can be made by various techniques, including but not limited to proteolytic digestion of an intact antibody as well as production by recombinant host cells (e.g. E. coli or phage), as described herein.

Chimeric and Humanized Antibodies

In certain embodiments, an antibody described herein is a chimeric antibody. Certain chimeric antibodies are described, e.g., in U.S. Pat. No. 4,816,567; and Morrison et al., Proc. Natl. Acad. Sci. USA, 81:6851-6855 (1984)). In one example, a chimeric antibody comprises a non-human variable region (e.g., a variable region derived from a mouse, rat, hamster, rabbit, or non-human primate, such as a monkey) and a human constant region. In a further example, a chimeric antibody is a “class switched” antibody in which the class or subclass has been changed from that of the parent antibody. Chimeric antibodies include antigen-binding fragments thereof.

In certain embodiments, a chimeric antibody is a humanized antibody. Typically, a non-human antibody is humanized to reduce immunogenicity to humans, while retaining the specificity and affinity of the parental non-human antibody. Generally, a humanized antibody comprises one or more variable domains in which HVRs, e.g., CDRs, (or portions thereof) are derived from a non-human antibody, and FRs (or portions thereof) are derived from human antibody sequences. A humanized antibody optionally will also comprise at least a portion of a human constant region. In some embodiments, some FR residues in a humanized antibody are substituted with corresponding residues from a non-human antibody (e.g., the antibody from which the HVR residues are derived), e.g., to restore or improve antibody specificity or affinity.

Humanized antibodies and methods of making them are reviewed, e.g., in Almagro and Fransson, Front. Biosci. 13:1619-1633 (2008), and are further described, e.g., in Riechmann et al., Nature 332:323-329 (1988); Queen et al., Proc. Nat'l Acad. Sci. USA 86:10029-10033 (1989); U.S. Pat. Nos. 5,821,337, 7,527,791, 6,982,321, and 7,087,409; Kashmiri et al., Methods 36:25-34 (2005) (describing SDR (a-CDR) grafting); Padlan, Mol. Immunol. 28:489-498 (1991) (describing “resurfacing”); Dall'Acqua et al., Methods 36:43-60 (2005) (describing “FR shuffling”); and Osbourn et al., Methods 36:61-68 (2005) and Klimka et al., Br. J. Cancer, 83:252-260 (2000) (describing the “guided selection” approach to FR shuffling).

Human framework regions that may be used for humanization include but are not limited to: framework regions selected using the “best-fit” method (see, e.g., Sims et al. J. Immunol. 151:2296 (1993)); framework regions derived from the consensus sequence of human antibodies of a particular subgroup of light or heavy chain variable regions (see, e.g., Carter et al. Proc. Natl. Acad. Sci. USA, 89:4285 (1992); and Presta et al. J. Immunol., 151:2623 (1993)); human mature (somatically mutated) framework regions or human germline framework regions (see, e.g., Almagro and Fransson, Front. Biosci. 13:1619-1633 (2008)); and framework regions derived from screening FR libraries (see, e.g., Baca et al., J. Biol. Chem. 272:10678-10684 (1997) and Rosok et al., J. Biol. Chem. 271:22611-22618 (1996)).

Human Antibodies

In certain embodiments, an antibody described herein is a human antibody. Human antibodies can be produced using various techniques known in the art. Human antibodies are described generally in van Dijk and van de Winkel, Curr. Opin. Pharmacol. 5: 368-74 (2001) and Lonberg, Curr. Opin. Immunol. 20:450-459 (2008).

Human antibodies may be prepared by administering an immunogen to a transgenic animal that has been modified to produce intact human antibodies or intact antibodies with human variable regions in response to antigenic challenge. Such animals typically contain all or a portion of the human immunoglobulin loci, which replace the endogenous immunoglobulin loci, or which are present extrachromosomally or integrated randomly into the animal's chromosomes. In such transgenic mice, the endogenous immunoglobulin loci have generally been inactivated. For review of methods for obtaining human antibodies from transgenic animals, see Lonberg, Nat. Biotech. 23:1117-1125 (2005). See also, e.g., U.S. Pat. Nos. 6,075,181 and 6,150,584 describing XENOMOUSE™ technology; U.S. Pat. No. 5,770,429 describing HUMAB® technology; U.S. Pat. No. 7,041,870 describing K-M MOUSE® technology, and U.S. Patent Application Publication No. US 2007/0061900, describing VELOCIMOUSE® technology). Human variable regions from intact antibodies generated by such animals may be further modified, e.g., by combining with a different human constant region.

Human antibodies can also be made by hybridoma-based methods. Human myeloma and mouse-human heteromyeloma cell lines for the production of human monoclonal antibodies have been described. (See, e.g., Kozbor J. Immunol., 133: 3001 (1984); Brodeur et al., Monoclonal Antibody Production Techniques and Applications, pp. 51-63 (Marcel Dekker, Inc., New York, 1987); and Boerner et al., J. Immunol., 147: 86 (1991).) Human antibodies generated via human B-cell hybridoma technology are also described in Li et al., Proc. Natl. Acad. Sci. USA, 103:3557-3562 (2006). Additional methods include those described, for example, in U.S. Pat. No. 7,189,826 (describing production of monoclonal human IgM antibodies from hybridoma cell lines) and Ni, Xiandai Mianyixue, 26(4):265-268 (2006) (describing human-human hybridomas). Human hybridoma technology (Trioma technology) is also described in Vollmers and Brandlein, Histology and Histopathology, 20(3):927-937 (2005) and Vollmers and Brandlein, Methods and Findings in Experimental and Clinical Pharmacology, 27(3):185-91 (2005).

Human antibodies may also be generated by isolating Fv clone variable domain sequences selected from human-derived phage display libraries. Such variable domain sequences may then be combined with a desired human constant domain. Techniques for selecting human antibodies from antibody libraries are described below.

Library-Derived Antibodies

Antibodies may be isolated by screening combinatorial libraries for antibodies with the desired activity or activities. For example, a variety of methods are known in the art for generating phage display libraries and screening such libraries for antibodies possessing the desired binding characteristics. Such methods are reviewed, e.g., in Hoogenboom et al. in Methods in Molecular Biology 178:1-37 (O'Brien et al., ed., Human Press, Totowa, N.J., 2001) and further described, e.g., in the McCafferty et al., Nature 348:552-554; Clackson et al., Nature 352: 624-628 (1991); Marks et al., J. Mol. Biol. 222: 581-597 (1992); Marks and Bradbury, in Methods in Molecular Biology 248:161-175 (Lo, ed., Human Press, Totowa, N.J., 2003); Sidhu et al., J. Mol. Biol. 338(2): 299-310 (2004); Lee et al., J. Mol. Biol. 340(5): 1073-1093 (2004); Fellouse, Proc. Natl. Acad. Sci. USA 101(34): 12467-12472 (2004); and Lee et al., J. Immunol. Methods 284(1-2): 119-132(2004).

In certain phage display methods, repertoires of VH and VL genes are separately cloned by polymerase chain reaction (PCR) and recombined randomly in phage libraries, which can then be screened for antigen-binding phage as described in Winter et al., Ann. Rev. Immunol., 12: 433-455 (1994). Phage typically display antibody fragments, either as single-chain Fv (scFv) fragments or as Fab fragments. Libraries from immunized sources provide high-affinity antibodies to the immunogen without the requirement of constructing hybridomas. Alternatively, the naive repertoire can be cloned (e.g., from human) to provide a single source of antibodies to a wide range of non-self and also self antigens without any immunization as described by Griffiths et al., EMBO J, 12: 725-734 (1993). Finally, naive libraries can also be made synthetically by cloning unrearranged V-gene segments from stem cells, and using PCR primers containing random sequence to encode the highly variable CDR3 regions and to accomplish rearrangement in vitro, as described by Hoogenboom and Winter, J. Mol. Biol., 227: 381-388 (1992). Patent publications describing human antibody phage libraries include, for example: U.S. Pat. No. 5,750,373, and US Patent Publication Nos. 2005/0079574, 2005/0119455, 2005/0266000, 2007/0117126, 2007/0160598, 2007/0237764, 2007/0292936, and 2009/0002360.

Antibodies or antibody fragments isolated from human antibody libraries are considered human antibodies or human antibody fragments herein.

Multispecific Antibodies

In certain embodiments, an antibody described herein is a multispecific antibody, e.g. a bispecific antibody. Multispecific antibodies are monoclonal antibodies that have binding specificities for at least two different sites. In some embodiments, one of the binding specificities is for a PD-1 axis component (e.g., PD-1, PDL1, or PDL2) and the other is for any other antigen. In some embodiments, one of the binding specificities is for IL-17 or IL-17R and the other is for any other antigen. In certain embodiments, bispecific antibodies may bind to two different epitopes of a PD-1 axis component (e.g., PD-1, PDL1, or PDL2), IL-17, or IL-17R. Bispecific antibodies can be prepared as full length antibodies or antibody fragments.

In some embodiments, one of the binding specificities is for a PD-1 axis component (e.g., PD-1, PDL1, or PDL2) and the other is for IL-17 or IL-17R. Provided herein are methods for treating or delaying progression of cancer in an individual comprising administering to the individual an effective amount of a multispecific antibody, wherein the multispecific antibody comprises a first binding specificity for a PD-1 axis component (e.g., PD-1, PDL1, or PDL2) and a second binding specificity for IL-17 or IL-17R. In some embodiments, a multispecific antibody may be made by any of the techniques described herein and below.

In some embodiments, one of the binding specificities is for IL-17A and the other is for IL-17F. Provided herein are methods for treating or delaying progression of cancer in an individual comprising administering to the individual an effective amount of a multispecific antibody, wherein the multispecific antibody comprises a first binding specificity for IL-17A and a second binding specificity for IL-17F. In some embodiments, one or both of the binding specificities are cross-reactive for IL-17A and IL-17F. In some embodiments, a multispecific antibody may be made by any of the techniques described herein and below.

Techniques for making multispecific antibodies include, but are not limited to, recombinant co-expression of two immunoglobulin heavy chain-light chain pairs having different specificities (see Milstein and Cuello, Nature 305: 537 (1983)), WO 93/08829, and Traunecker et al., EMBO J. 10: 3655 (1991)), and “knob-in-hole” engineering (see, e.g., U.S. Pat. No. 5,731,168). Multi-specific antibodies may also be made by engineering electrostatic steering effects for making antibody Fc-heterodimeric molecules (WO 2009/089004A1); cross-linking two or more antibodies or fragments (see, e.g., U.S. Pat. No. 4,676,980, and Brennan et al., Science, 229: 81 (1985)); using leucine zippers to produce bi-specific antibodies (see, e.g., Kostelny et al., J. Immunol., 148(5):1547-1553 (1992)); using “diabody” technology for making bispecific antibody fragments (see, e.g., Hollinger et al., Proc. Natl. Acad. Sci. USA, 90:6444-6448 (1993)); and using single-chain Fv (sFv) dimers (see, e.g. Gruber et al., J. Immunol., 152:5368 (1994)); and preparing trispecific antibodies as described, e.g., in Tutt et al. J. Immunol. 147: 60 (1991).

Engineered antibodies with three or more functional antigen binding sites, including “Octopus antibodies,” are also included herein (see, e.g. US 2006/0025576A1).

The antibody or fragment herein also includes a “Dual Acting FAb” or “DAF” comprising an antigen binding site that binds to a PD-1 axis component (e.g., PD-1, PDL1, or PDL2), IL-17, or IL-17R as well as another, different antigen (see, US 2008/0069820, for example).

Antibody Variants

In certain embodiments, amino acid sequence variants of the antibodies described herein are contemplated. For example, it may be desirable to improve the binding affinity and/or other biological properties of the antibody Amino acid sequence variants of an antibody may be prepared by introducing appropriate modifications into the nucleotide sequence encoding the antibody, or by peptide synthesis. Such modifications include, for example, deletions from, and/or insertions into and/or substitutions of residues within the amino acid sequences of the antibody. Any combination of deletion, insertion, and substitution can be made to arrive at the final construct, provided that the final construct possesses the desired characteristics, e.g., antigen-binding.

Substitution, Insertion, and Deletion Variants

In certain embodiments, antibody variants having one or more amino acid substitutions are described. Sites of interest for substitutional mutagenesis include the HVRs and FRs. Conservative substitutions are shown in Table 1 under the heading of “conservative substitutions.” More substantial changes are provided in Table 1 under the heading of “exemplary substitutions,” and as further described below in reference to amino acid side chain classes. Amino acid substitutions may be introduced into an antibody of interest and the products screened for a desired activity, e.g., retained/improved antigen binding, decreased immunogenicity, or improved ADCC or CDC.

TABLE 1

| Original | | Preferred |
|---|---|---|
| Residue | Exemplary Substitutions | Substitutions |
| Ala (A) | Val; Leu; Ile | Val |
| Arg (R) | Lys; Gln; Asn | Lys |
| Asn (N) | Gln; His; Asp, Lys; Arg | Gln |
| Asp (D) | Glu; Asn | Glu |
| Cys (C) | Ser; Ala | Ser |
| Gln (Q) | Asn; Glu | Asn |
| Glu (E) | Asp; Gln | Asp |
| Gly (G) | Ala | Ala |
| His (H) | Asn; Gln; Lys; Arg | Arg |
| Ile (I) | Leu; Val; Met; Ala; Phe; Norleucine | Leu |
| Leu (L) | Norleucine; Ile; Val; Met; Ala; Phe | Ile |
| Lys (K) | Arg; Gln; Asn | Arg |
| Met (M) | Leu; Phe; Ile | Leu |
| Phe (F) | Trp; Leu; Val; Ile; Ala; Tyr | Tyr |
| Pro (P) | Ala | Ala |
| Ser (S) | Thr | Thr |
| Thr (T) | Val; Ser | Ser |
| Trp (W) | Tyr; Phe | Tyr |
| Tyr (Y) | Trp; Phe; Thr; Ser | Phe |
| Val (V) | Ile; Leu; Met; Phe; Ala; Norleucine | Leu |

Amino acids may be grouped according to common side-chain properties:

a. hydrophobic: Norleucine, Met, Ala, Val, Leu, Ile;

b. neutral hydrophilic: Cys, Ser, Thr, Asn, Gln;

c. acidic: Asp, Glu;

d. basic: His, Lys, Arg;

e. residues that influence chain orientation: Gly, Pro;

f. aromatic: Trp, Tyr, Phe.

Non-conservative substitutions will entail exchanging a member of one of these classes for another class.

One type of substitutional variant involves substituting one or more hypervariable region residues of a parent antibody (e.g. a humanized or human antibody). Generally, the resulting variant(s) selected for further study will have modifications (e.g., improvements) in certain biological properties (e.g., increased affinity, reduced immunogenicity) relative to the parent antibody and/or will have substantially retained certain biological properties of the parent antibody. An exemplary substitutional variant is an affinity matured antibody, which may be conveniently generated, e.g., using phage display-based affinity maturation techniques such as those described herein. Briefly, one or more HVR residues are mutated and the variant antibodies displayed on phage and screened for a particular biological activity (e.g. binding affinity).

Alterations (e.g., substitutions) may be made in HVRs, e.g., to improve antibody affinity. Such alterations may be made in HVR “hotspots,” i.e., residues encoded by codons that undergo mutation at high frequency during the somatic maturation process (see, e.g., Chowdhury, Methods Mol. Biol. 207:179-196 (2008)), and/or SDRs (a-CDRs), with the resulting variant VH or VL being tested for binding affinity. Affinity maturation by constructing and reselecting from secondary libraries has been described, e.g., in Hoogenboom et al. in Methods in Molecular Biology 178:1-37 (O'Brien et al., ed., Human Press, Totowa, N.J., (2001).) In some embodiments of affinity maturation, diversity is introduced into the variable genes chosen for maturation by any of a variety of methods (e.g., error-prone PCR, chain shuffling, or oligonucleotide-directed mutagenesis). A secondary library is then created. The library is then screened to identify any antibody variants with the desired affinity. Another method to introduce diversity involves HVR-directed approaches, in which several HVR residues (e.g., 4-6 residues at a time) are randomized HVR residues involved in antigen binding may be specifically identified, e.g., using alanine scanning mutagenesis or modeling. CDR-H3 and CDR-L3 in particular are often targeted.

In certain embodiments, substitutions, insertions, or deletions may occur within one or more HVRs so long as such alterations do not substantially reduce the ability of the antibody to bind antigen. For example, conservative alterations (e.g., conservative substitutions as provided herein) that do not substantially reduce binding affinity may be made in HVRs. Such alterations may be outside of HVR “hotspots” or SDRs. In certain embodiments of the variant VH and VL sequences provided above, each HVR either is unaltered, or contains no more than one, two or three amino acid substitutions.

A useful method for identification of residues or regions of an antibody that may be targeted for mutagenesis is called “alanine scanning mutagenesis” as described by Cunningham and Wells (1989) Science, 244:1081-1085. In this method, a residue or group of target residues (e.g., charged residues such as arg, asp, his, lys, and glu) are identified and replaced by a neutral or negatively charged amino acid (e.g., alanine or polyalanine) to determine whether the interaction of the antibody with antigen is affected. Further substitutions may be introduced at the amino acid locations demonstrating functional sensitivity to the initial substitutions. Alternatively, or additionally, a crystal structure of an antigen-antibody complex to identify contact points between the antibody and antigen. Such contact residues and neighboring residues may be targeted or eliminated as candidates for substitution. Variants may be screened to determine whether they contain the desired properties.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an antibody with an N-terminal methionyl residue. Other insertional variants of the antibody molecule include the fusion to the N- or C-terminus of the antibody to an enzyme (e.g. for ADEPT) or a polypeptide which increases the serum half-life of the antibody.

Glycosylation Variants

In certain embodiments, an antibody described herein is altered to increase or decrease the extent to which the antibody is glycosylated. Addition or deletion of glycosylation sites to an antibody may be conveniently accomplished by altering the amino acid sequence such that one or more glycosylation sites is created or removed.

Where the antibody comprises an Fc region, the carbohydrate attached thereto may be altered. Native antibodies produced by mammalian cells typically comprise a branched, biantennary oligosaccharide that is generally attached by an N-linkage to Asn297 of the CH2 domain of the Fc region. See, e.g., Wright et al. TIBTECH 15:26-32 (1997). The oligosaccharide may include various carbohydrates, e.g., mannose, N-acetyl glucosamine (GlcNAc), galactose, and sialic acid, as well as a fucose attached to a GlcNAc in the “stem” of the biantennary oligosaccharide structure. In some embodiments, modifications of the oligosaccharide in an antibody of the invention may be made in order to create antibody variants with certain improved properties.

In one embodiment, antibody variants are provided comprising an Fc region wherein a carbohydrate structure attached to the Fc region has reduced fucose or lacks fucose, which may improve ADCC function. Specifically, antibodies are contemplated herein that have reduced fusose relative to the amount of fucose on the same antibody produced in a wild-type CHO cell. That is, they are characterized by having a lower amount of fucose than they would otherwise have if produced by native CHO cells (e.g., a CHO cell that produce a native glycosylation pattern, such as, a CHO cell containing a native FUT8 gene). In certain embodiments, the antibody is one wherein less than about 50%, 40%, 30%, 20%, 10%, or 5% of the N-linked glycans thereon comprise fucose. For example, the amount of fucose in such an antibody may be from 1% to 80%, from 1% to 65%, from 5% to 65% or from 20% to 40%. In certain embodiments, the antibody is one wherein none of the N-linked glycans thereon comprise fucose, i.e., wherein the antibody is completely without fucose, or has no fucose or is afucosylated. The amount of fucose is determined by calculating the average amount of fucose within the sugar chain at Asn297, relative to the sum of all glycostructures attached to Asn 297 (e. g. complex, hybrid and high mannose structures) as measured by MALDI-TOF mass spectrometry, as described in WO 2008/077546, for example. Asn297 refers to the asparagine residue located at about position 297 in the Fc region (Eu numbering of Fc region residues); however, Asn297 may also be located about ±3 amino acids upstream or downstream of position 297, i.e., between positions 294 and 300, due to minor sequence variations in antibodies. Such fucosylation variants may have improved ADCC function. See, e.g., US Patent Publication Nos. US 2003/0157108 (Presta, L.); US 2004/0093621 (Kyowa Hakko Kogyo Co., Ltd). Examples of publications related to “defucosylated” or “fucose-deficient” antibody variants include: US 2003/0157108; WO 2000/61739; WO 2001/29246; US 2003/0115614; US 2002/0164328; US 2004/0093621; US 2004/0132140; US 2004/0110704; US 2004/0110282; US 2004/0109865; WO 2003/085119; WO 2003/084570; WO 2005/035586; WO 2005/035778; WO2005/053742; WO2002/031140; Okazaki et al. J. Mol. Biol. 336:1239-1249 (2004); Yamane-Ohnuki et al. Biotech. Bioeng. 87: 614 (2004). Examples of cell lines capable of producing defucosylated antibodies include Lec13 CHO cells deficient in protein fucosylation (Ripka et al. Arch. Biochem. Biophys. 249:533-545 (1986); US Pat Appl No US 2003/0157108 A1, Presta, L; and WO 2004/056312 A1, Adams et al., especially at Example 11), and knockout cell lines, such as alpha-1,6-fucosyltransferase gene, FUT8, knockout CHO cells (see, e.g., Yamane-Ohnuki et al. Biotech. Bioeng. 87: 614 (2004); Kanda, Y. et al., Biotechnol. Bioeng., 94(4):680-688 (2006); and WO2003/085107).

Antibody variants are further provided with bisected oligosaccharides, e.g., in which a biantennary oligosaccharide attached to the Fc region of the antibody is bisected by GlcNAc. Such antibody variants may have reduced fucosylation and/or improved ADCC function. Examples of such antibody variants are described, e.g., in WO 2003/011878 (Jean-Mairet et al.); U.S. Pat. No. 6,602,684 (Umana et al.); US 2005/0123546 (Umana et al.), and Ferrara et al., Biotechnology and Bioengineering, 93(5): 851-861 (2006). Antibody variants with at least one galactose residue in the oligosaccharide attached to the Fc region are also provided. Such antibody variants may have improved CDC function. Such antibody variants are described, e.g., in WO 1997/30087 (Patel et al.); WO 1998/58964 (Raju, S.); and WO 1999/22764 (Raju, S.).

In certain embodiments, the antibody variants comprising an Fc region described herein are capable of binding to an FcγRIII. In certain embodiments, the antibody variants comprising an Fc region described herein have ADCC activity in the presence of human effector cells or have increased ADCC activity in the presence of human effector cells compared to the otherwise same antibody comprising a human wild-type IgG1Fc region.

Fc Region Variants

In certain embodiments, one or more amino acid modifications may be introduced into the Fc region of an antibody described herein, thereby generating an Fc region variant. The Fc region variant may comprise a human Fc region sequence (e.g., a human IgG1, IgG2, IgG3 or IgG4 Fc region) comprising an amino acid modification (e.g. a substitution) at one or more amino acid positions.

In certain embodiments, the invention contemplates an antibody variant that possesses some but not all effector functions, which make it a desirable candidate for applications in which the half life of the antibody in vivo is important yet certain effector functions (such as complement and ADCC) are unnecessary or deleterious. In vitro and/or in vivo cytotoxicity assays can be conducted to confirm the reduction/depletion of CDC and/or ADCC activities. For example, Fc receptor (FcR) binding assays can be conducted to ensure that the antibody lacks FcγR binding (hence likely lacking ADCC activity), but retains FcRn binding ability. The primary cells for mediating ADCC, NK cells, express Fc(RIII only, whereas monocytes express Fc(RI, Fc(RII and Fc(RIII. FcR expression on hematopoietic cells is summarized in Table 3 on page 464 of Ravetch and Kinet, Annu. Rev. Immunol. 9:457-492 (1991). Non-limiting examples of in vitro assays to assess ADCC activity of a molecule of interest is described in U.S. Pat. No. 5,500,362 (see, e.g. Hellstrom, I. et al. Proc. Nat'l Acad. Sci. USA 83:7059-7063 (1986)) and Hellstrom, I et al., Proc. Nat'l Acad. Sci. USA 82:1499-1502 (1985); U.S. Pat. No. 5,821,337 (see Bruggemann, M. et al., J. Exp. Med. 166:1351-1361 (1987)). Alternatively, non-radioactive assays methods may be employed (see, for example, ACTI™ non-radioactive cytotoxicity assay for flow cytometry (CellTechnology, Inc. Mountain View, Calif.; and CytoTox 96® non-radioactive cytotoxicity assay (Promega, Madison, Wis.). Useful effector cells for such assays include peripheral blood mononuclear cells (PBMC) and Natural Killer (NK) cells. Alternatively, or additionally, ADCC activity of the molecule of interest may be assessed in vivo, e.g., in an animal model such as that disclosed in Clynes et al. Proc. Nat'l Acad. Sci. USA 95:652-656 (1998). C1q binding assays may also be carried out to confirm that the antibody is unable to bind C1q and hence lacks CDC activity. See, e.g., C1q and C3c binding ELISA in WO 2006/029879 and WO 2005/100402. To assess complement activation, a CDC assay may be performed (see, for example, Gazzano-Santoro et al., J. Immunol. Methods 202:163 (1996); Cragg, M. S. et al., Blood 101:1045-1052 (2003); and Cragg, M. S. and M. J. Glennie, Blood 103:2738-2743 (2004)). FcRn binding and in vivo clearance/half life determinations can also be performed using methods known in the art (see, e.g., Petkova, S. B. et al., Int'l. Immunol. 18(12):1759-1769 (2006)).

Antibodies with reduced effector function include those with substitution of one or more of Fc region residues 238, 265, 269, 270, 297, 327 and 329 (U.S. Pat. No. 6,737,056). Such Fc mutants include Fc mutants with substitutions at two or more of amino acid positions 265, 269, 270, 297 and 327, including the so-called “DANA” Fc mutant with substitution of residues 265 and 297 to alanine (U.S. Pat. No. 7,332,581).

Certain antibody variants with improved or diminished binding to FcRs are described. (See, e.g., U.S. Pat. No. 6,737,056; WO 2004/056312, and Shields et al., J. Biol. Chem. 9(2): 6591-6604 (2001).)

In some embodiments, alterations are made in the Fc region that result in altered (i.e., either improved or diminished) C1q binding and/or Complement Dependent Cytotoxicity (CDC), e.g., as described in U.S. Pat. No. 6,194,551, WO 99/51642, and Idusogie et al. J. Immunol. 164: 4178-4184 (2000).

Antibodies with increased half lives and improved binding to the neonatal Fc receptor (FcRn), which is responsible for the transfer of maternal IgGs to the fetus (Guyer et al., J. Immunol. 117:587 (1976) and Kim et al., J. Immunol. 24:249 (1994)), are described in US2005/0014934A1 (Hinton et al.)). Those antibodies comprise an Fc region with one or more substitutions therein which improve binding of the Fc region to FcRn. Such Fc variants include those with substitutions at one or more of Fc region residues: 238, 256, 265, 272, 286, 303, 305, 307, 311, 312, 317, 340, 356, 360, 362, 376, 378, 380, 382, 413, 424 or 434, e.g., substitution of Fc region residue 434 (U.S. Pat. No. 7,371,826).

See also Duncan & Winter, Nature 322:738-40 (1988); U.S. Pat. No. 5,648,260; U.S. Pat. No. 5,624,821; and WO 94/29351 concerning other examples of Fc region variants.

Cysteine Engineered Antibody Variants

In certain embodiments, it may be desirable to create cysteine engineered antibodies, e.g., “thioMAbs,” in which one or more residues of an antibody are substituted with cysteine residues. In particular embodiments, the substituted residues occur at accessible sites of the antibody. By substituting those residues with cysteine, reactive thiol groups are thereby positioned at accessible sites of the antibody and may be used to conjugate the antibody to other moieties, such as drug moieties or linker-drug moieties, to create an immunoconjugate, as described further herein. In certain embodiments, any one or more of the following residues may be substituted with cysteine: V205 (Kabat numbering) of the light chain; A118 (EU numbering) of the heavy chain; and S400 (EU numbering) of the heavy chain Fc region. Cysteine engineered antibodies may be generated as described, e.g., in U.S. Pat. No. 7,521,541.

Antibody Derivatives

In certain embodiments, an antibody described herein may be further modified to contain additional nonproteinaceous moieties that are known in the art and readily available. The moieties suitable for derivatization of the antibody include but are not limited to water soluble polymers. Non-limiting examples of water soluble polymers include, but are not limited to, polyethylene glycol (PEG), copolymers of ethylene glycol/propylene glycol, carboxymethylcellulose, dextran, polyvinyl alcohol, polyvinyl pyrrolidone, poly-1, 3-dioxolane, poly-1,3,6-trioxane, ethylene/maleic anhydride copolymer, polyaminoacids (either homopolymers or random copolymers), and dextran or poly(n-vinyl pyrrolidone)polyethylene glycol, propropylene glycol homopolymers, prolypropylene oxide/ethylene oxide co-polymers, polyoxyethylated polyols (e.g., glycerol), polyvinyl alcohol, and mixtures thereof. Polyethylene glycol propionaldehyde may have advantages in manufacturing due to its stability in water. The polymer may be of any molecular weight, and may be branched or unbranched. The number of polymers attached to the antibody may vary, and if more than one polymer are attached, they can be the same or different molecules. In general, the number and/or type of polymers used for derivatization can be determined based on considerations including, but not limited to, the particular properties or functions of the antibody to be improved, whether the antibody derivative will be used in a therapy under defined conditions, etc.

In another embodiment, conjugates of an antibody and nonproteinaceous moiety that may be selectively heated by exposure to radiation are provided. In one embodiment, the nonproteinaceous moiety is a carbon nanotube (Kam et al., Proc. Natl. Acad. Sci. USA 102: 11600-11605 (2005)). The radiation may be of any wavelength, and includes, but is not limited to, wavelengths that do not harm ordinary cells, but which heat the nonproteinaceous moiety to a temperature at which cells proximal to the antibody-nonproteinaceous moiety are killed.

Recombinant Methods and Compositions

Antibodies may be produced using recombinant methods and compositions, e.g., as described in U.S. Pat. No. 4,816,567. An isolated nucleic acid encoding an antibody may encode an amino acid sequence comprising the VL and/or an amino acid sequence comprising the VH of the antibody (e.g., the light and/or heavy chains of the antibody). One or more vectors (e.g., expression vectors) comprising such nucleic acids may be used. A host cell comprising such nucleic acid is provided. In one such embodiment, a host cell comprises (e.g., has been transformed with): (1) a vector comprising a nucleic acid that encodes an amino acid sequence comprising the VL of the antibody and an amino acid sequence comprising the VH of the antibody, or (2) a first vector comprising a nucleic acid that encodes an amino acid sequence comprising the VL of the antibody and a second vector comprising a nucleic acid that encodes an amino acid sequence comprising the VH of the antibody. In one embodiment, the host cell is eukaryotic, e.g. a Chinese Hamster Ovary (CHO) cell or lymphoid cell (e.g., Y0, NS0, Sp20 cell). A method of making an antibody may include culturing a host cell comprising a nucleic acid encoding the antibody, as provided above, under conditions suitable for expression of the antibody, and optionally recovering the antibody from the host cell (or host cell culture medium).

For recombinant production of an antibody, nucleic acid encoding an antibody, e.g., as described above, is isolated and inserted into one or more vectors for further cloning and/or expression in a host cell. Such nucleic acid may be readily isolated and sequenced using conventional procedures (e.g., by using oligonucleotide probes that are capable of binding specifically to genes encoding the heavy and light chains of the antibody).

Suitable host cells for cloning or expression of antibody-encoding vectors include prokaryotic or eukaryotic cells described herein. For example, antibodies may be produced in bacteria, in particular when glycosylation and Fc effector function are not needed. For expression of antibody fragments and polypeptides in bacteria, see, e.g., U.S. Pat. Nos. 5,648,237, 5,789,199, and 5,840,523. (See also Charlton, Methods in Molecular Biology, Vol. 248 (B. K. C. Lo, ed., Humana Press, Totowa, N.J., 2003), pp. 245-254, describing expression of antibody fragments in E. coli.) After expression, the antibody may be isolated from the bacterial cell paste in a soluble fraction and can be further purified.

In addition to prokaryotes, eukaryotic microbes such as filamentous fungi or yeast are suitable cloning or expression hosts for antibody-encoding vectors, including fungi and yeast strains whose glycosylation pathways have been “humanized,” resulting in the production of an antibody with a partially or fully human glycosylation pattern. See Gerngross, Nat. Biotech. 22:1409-1414 (2004), and Li et al., Nat. Biotech. 24:210-215 (2006).

Suitable host cells for the expression of glycosylated antibody are also derived from multicellular organisms (invertebrates and vertebrates). Examples of invertebrate cells include plant and insect cells. Numerous baculoviral strains have been identified which may be used in conjunction with insect cells, particularly for transfection of Spodoptera frugiperda cells.

Plant cell cultures can also be utilized as hosts. See, e.g., U.S. Pat. Nos. 5,959,177, 6,040,498, 6,420,548, 7,125,978, and 6,417,429 (describing PLANTIBODIES™ technology for producing antibodies in transgenic plants).

Vertebrate cells may also be used as hosts. For example, mammalian cell lines that are adapted to grow in suspension may be useful. Other examples of useful mammalian host cell lines are monkey kidney CV1 line transformed by SV40 (COS-7); human embryonic kidney line (293 or 293 cells as described, e.g., in Graham et al., J. Gen Virol. 36:59 (1977)); baby hamster kidney cells (BHK); mouse sertoli cells (TM4 cells as described, e.g., in Mather, Biol. Reprod. 23:243-251 (1980)); monkey kidney cells (CV1); African green monkey kidney cells (VERO-76); human cervical carcinoma cells (HELA); canine kidney cells (MDCK; buffalo rat liver cells (BRL 3A); human lung cells (W138); human liver cells (Hep G2); mouse mammary tumor (MMT 060562); TRI cells, as described, e.g., in Mather et al., Annals N.Y. Acad. Sci. 383:44-68 (1982); MRC 5 cells; and FS4 cells. Other useful mammalian host cell lines include Chinese hamster ovary (CHO) cells, including DHFR⁻ CHO cells (Urlaub et al., Proc. Natl. Acad. Sci. USA 77:4216 (1980)); and myeloma cell lines such as Y0, NS0 and Sp2/0. For a review of certain mammalian host cell lines suitable for antibody production, see, e.g., Yazaki and Wu, Methods in Molecular Biology, Vol. 248 (B. K. C. Lo, ed., Humana Press, Totowa, N.J.), pp. 255-268 (2003).

Assays

Antibodies described herein may be identified, screened for, or characterized for their physical/chemical properties and/or biological activities by various assays known in the art.

In one aspect, an antibody may be tested for its antigen binding activity, e.g., by known methods such as ELISA, Western blot, etc.

In another aspect, competition assays may be used to identify an antibody that competes with an antibody that specifically binds an epitope (e.g., an epitope derived from a PD-1 axis component such as PD-1, PDL1, or PDL2; IL-17; or IL-17R). In certain embodiments, such a competing antibody binds to the same epitope (e.g., a linear or a conformational epitope) that is bound by an antibody that specifically binds an epitope (e.g., an epitope derived from a PD-1 axis component such as PD-1, PDL1, or PDL2; IL-17; or IL-17R). Detailed exemplary methods for mapping an epitope to which an antibody binds are provided in Morris (1996) “Epitope Mapping Protocols,” in Methods in Molecular Biology vol. 66 (Humana Press, Totowa, N.J.).

In an exemplary competition assay, immobilized antibody that specifically binds an epitope (e.g., an epitope derived from a PD-1 axis component such as PD-1, PDL1, or PDL2; IL-17; or IL-17R) or cells expressing an antibody that specifically binds an epitope (e.g., an epitope derived from a PD-1 axis component such as PD-1, PDL1, or PDL2; IL-17; or IL-17R) on its cell surface are incubated in a solution comprising a first labeled antibody that specifically binds the epitope (e.g., an epitope derived from a PD-1 axis component such as PD-1, PDL1, or PDL2; IL-17; or IL-17R) and a second unlabeled antibody that is being tested for its ability to compete with the first antibody for binding to the epitope. The second antibody may be present in a hybridoma supernatant. As a control, immobilized antibody that specifically binds an epitope (e.g., an epitope derived from a PD-1 axis component such as PD-1, PDL1, or PDL2; IL-17; or IL-17R) or cells expressing an antibody that specifically binds an epitope (e.g., an epitope derived from a PD-1 axis component such as PD-1, PDL1, or PDL2; IL-17; or IL-17R) is incubated in a solution comprising the first labeled antibody but not the second unlabeled antibody. After incubation under conditions permissive for binding of the first antibody to the epitope, excess unbound antibody is removed, and the amount of label associated with immobilized antibody that specifically binds the epitope or cells expressing the antibody that specifically binds the epitope is measured. If the amount of label associated with immobilized antibody that specifically binds the epitope or cells expressing the antibody that specifically binds the epitope is substantially reduced in the test sample relative to the control sample, then that indicates that the second antibody is competing with the first antibody for binding to the epitope. See Harlow and Lane (1988) Antibodies: A Laboratory Manual ch.14 (Cold Spring Harbor Laboratory, Cold Spring Harbor, N.Y.).

Activity Assays

Antibodies produced as described above may be subjected to one or more activity assays to select an antibody with beneficial properties from a therapeutic perspective or selecting formulations and conditions that retain biological activity of the antibody. The antibody may be tested for its ability to bind the antigen against which it was raised (e.g., as described above). For example, methods known in the art (such as ELISA, Western Blot, etc.) may be used.

For example, the antigen binding properties of an antibody can be evaluated in an assay that detects the ability of the antibody to specifically bind to a molecule containing an antibody epitope. In some embodiments, the binding of the antibody may be determined by saturation binding; ELISA; and/or competition assays (e.g. RIA's), for example. Also, the antibody may be subjected to other biological activity assays, e.g., in order to evaluate its effectiveness as a therapeutic. Such assays are known in the art and depend on the target antigen and intended use for the antibody. For example, the biological effects of PD-1 axis blockade or IL-17 blockade by the antibody can be assessed in an animal model, cell culture model, or an in vitro model of PD-1 and/or IL-17 signaling (for PD-1, see, e.g., as described in U.S. Pat. No. 8,217,149).

To screen for antibodies which bind to a particular epitope on the antigen of interest, a routine cross-blocking assay such as that described in Antibodies, A Laboratory Manual, Cold Spring Harbor Laboratory, Ed Harlow and David Lane (1988), can be performed. Alternatively, epitope mapping, e.g. as described in Champe et al., J. Biol. Chem. 270:1388-1394 (1995), can be performed to determine whether the antibody binds an epitope of interest.

### VI. Methods

In one aspect, provided herein are methods for treating or delaying progression of cancer in an individual comprising administering to the individual an effective amount of a PD-1 axis binding antagonist and an IL-17 binding antagonist. In another aspect, provided herein are methods for enhancing immune function in an individual having cancer comprising administering an effective amount of a combination of a PD-1 axis binding antagonist and an IL-17 binding antagonist. The methods of this disclosure may find use, inter alia, in treating conditions where enhanced immunogenicity is desired such as increasing tumor immunogenicity for the treatment of cancer or T cell dysfunctional disorders. A variety of cancers may be treated, or their progression may be delayed, by these methods.

In some embodiments, a cancer to be treated by the methods of the present disclosure includes, but is not limited to, colorectal cancer, renal cell cancer (e.g., renal cell carcinoma), melanoma, bladder cancer, ovarian cancer, breast cancer (e.g., triple-negative breast cancer, HER2-positive breast cancer, or hormone receptor-positive cancer), and non-small-cell lung cancer (e.g., squamous non-small-cell lung cancer or non-squamous non-small-cell lung cancer). In some embodiments, a cancer to be treated by the methods of the present disclosure includes, but is not limited to, a carcinoma, lymphoma, blastoma, sarcoma, and leukemia. In some embodiments, a cancer to be treated by the methods of the present disclosure includes, but is not limited to, squamous cell cancer, lung cancer (including small-cell lung cancer, non-small cell lung cancer, adenocarcinoma of the lung, and squamous carcinoma of the lung), melanoma, renal cell carcinoma, cancer of the peritoneum, hepatocellular cancer, gastric or stomach cancer (including gastrointestinal cancer), pancreatic cancer, glioblastoma, cervical cancer, ovarian cancer, liver cancer, bladder cancer, hepatoma, breast cancer, colon cancer, colorectal cancer, endometrial or uterine carcinoma, salivary gland carcinoma, kidney or renal cancer, liver cancer, prostate cancer, vulval cancer, thyroid cancer, hepatic carcinoma and various types of head and neck cancer, as well as B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia); chronic lymphocytic leukemia (CLL); acute lymphoblastic leukemia (ALL); Hairy cell leukemia; chronic myeloblastic leukemia; and post-transplant lymphoproliferative disorder (PTLD), as well as abnormal vascular proliferation associated with phakomatoses, edema (such as that associated with brain tumors), and Meigs' syndrome. In some embodiments, the cancer may be an early stage cancer or a late stage cancer. In some embodiments, the cancer may be a primary tumor. In some embodiments, the cancer may be a metastatic tumor at a second site derived from any of the above types of cancer.

In some embodiments, the individual has cancer or is at risk of developing cancer. In some embodiments, the treatment results in a sustained response in the individual after cessation of the treatment. In some embodiments, the individual has cancer that may be at early stage or late stage. In some embodiments, the individual is a human. In some embodiments, the individual is a mammal, such as domesticated animals (e.g., cows, sheep, cats, dogs, and horses), primates (e.g., humans and non-human primates such as monkeys), rabbits, and rodents (e.g., mice and rats).

In some embodiments, the combination therapy of the invention comprises administration of a PD-1 axis binding antagonist and an IL-17 binding antagonist. The PD-1 axis binding antagonist and the IL-17 binding antagonist may be administered in any suitable manner known in the art. For example, the PD-1 axis binding antagonist and the IL-17 binding antagonist may be administered sequentially (at different times) or concurrently (at the same time).

In some embodiments, the PD-1 axis binding antagonist or IL-17 binding antagonist is administered continuously. In some embodiments, the PD-1 axis binding antagonist or IL-17 binding antagonist is administered intermittently. In some embodiments, the IL-17 binding antagonist is administered before administration of the PD-1 axis binding antagonist. In some embodiments, the IL-17 binding antagonist is administered simultaneously with administration of the PD-1 axis binding antagonist (e.g., formulated in the same composition). In some embodiments, the IL-17 binding antagonist is administered after administration of the PD-1 axis binding antagonist. In some embodiments, the IL-17 binding antagonist is administered in the same day as the PD-1 axis binding antagonist. In some embodiments, the IL-17 binding antagonist is administered within 2 days, within 3 days, within 4 days, within 5 days, within 6 days, within 1 week, within 2 weeks, within 3 weeks, or within 1 month of administering the PD-1 axis binding antagonist.

In some embodiments, provided is a method for treating or delaying progression of cancer in an individual comprising administering to the individual an effective amount of a PD-1 axis binding antagonist and an IL-17 binding antagonist, and further comprising administering an additional therapy. The additional therapy may be radiation therapy, surgery (e.g., lumpectomy and a mastectomy), chemotherapy, gene therapy, DNA therapy, viral therapy, RNA therapy, immunotherapy, bone marrow transplantation, nanotherapy, monoclonal antibody therapy, or a combination of the foregoing. The additional therapy may be in the form of adjuvant or neoadjuvant therapy. In some embodiments, the additional therapy is the administration of small molecule enzymatic inhibitor or anti-metastatic agent. In some embodiments, the additional therapy is the administration of side-effect limiting agents (e.g., agents intended to lessen the occurrence and/or severity of side effects of treatment, such as anti-nausea agents, etc.). In some embodiments, the additional therapy is radiation therapy. In some embodiments, the additional therapy is surgery. In some embodiments, the additional therapy is a combination of radiation therapy and surgery. In some embodiments, the additional therapy is gamma irradiation. In some embodiments, the additional therapy is therapy targeting PI3K/AKT/mTOR pathway, HSP90 inhibitor, tubulin inhibitor, apoptosis inhibitor, and/or chemopreventative agent. The additional therapy may be one or more of the chemotherapeutic agents described hereabove.

The PD-1 axis binding antagonist and the IL-17 binding antagonist may be administered by the same route of administration or by different routes of administration. In some embodiments, the PD-1 axis binding antagonist is administered intravenously, intramuscularly, subcutaneously, topically, orally, transdermally, intraperitoneally, intraorbitally, by implantation, by inhalation, intrathecally, intraventricularly, or intranasally. In some embodiments, the IL-17 binding antagonist is administered intravenously, intramuscularly, subcutaneously, topically, orally, transdermally, intraperitoneally, intraorbitally, by implantation, by inhalation, intrathecally, intraventricularly, or intranasally. An effective amount of the PD-1 axis binding antagonist and the IL-17 binding antagonist may be administered for prevention or treatment of disease. The appropriate dosage of the PD-1 axis binding antagonist and/or the IL-17 binding antagonist may be determined based on the type of disease to be treated, the type of the PD-1 axis binding antagonist and the IL-17 binding antagonist, the severity and course of the disease, the clinical condition of the individual, the individual's clinical history and response to the treatment, and the discretion of the attending physician.

In some embodiments, the treatment comprising administering to the individual an effective amount of a PD-1 axis binding antagonist and an IL-17 binding antagonist (optionally further comprising administering an additional therapy as described above) results in a sustained response in the individual after cessation of the treatment.

In some embodiments, an individual is first treated according to a current standard of care for the cancer to be treated, then administered an effective amount of a PD-1 axis binding antagonist and an IL-17 binding antagonist (optionally further comprising administering an additional therapy as described above). Standards of care for any of the cancers described herein are known to persons of ordinary skill in clinical oncology. The methods described herein may find use, inter alia, in treating patients that are not responsive to current standard of care.

In some embodiments, a biopsy sample obtained from the cancer of the individual shows expression of IL-17. In some embodiments, a biopsy sample obtained from the cancer of the individual shows expression of an IL-17 gene signature (e.g., a group of genes or proteins whose expression is thought or predicted to be functionally related or correlated with IL-17 signaling, such as one or more genes selected from IL-17A, IL-17F, IL-8, CSF3, CXCL1, CXCL3, and CCL20). In some embodiments, a biopsy sample obtained from the cancer of the individual shows expression of an IL-17 gene signature (e.g., a group of genes or proteins whose expression is thought or predicted to be functionally related or correlated with IL-17 signaling, such as one or more genes selected from CD4, CD8a, IL17A, IL17B, IL17C, IL17D, IL17F, IL17RA, IL17RC, C3, CCL2, CCL20, CSF2, CSF3, CXCL1, CXCL2, CXCL3, CXCL5, CXCL10, CXCR1, CXCR2, ICAM1, IL6, IL8, MMP1, MMP2, MMP3, MMP8, MMP9, MMP13, MMP14, MMP25, NCF4, NFKBIZ, S100A8, S100A9, SAA2, SAA1, SAA3, SAA4, TIMP1, TIMP2, TIMP3, and TIMP4). In certain embodiments, the IL-17 gene signature includes one or more genes selected from NFKBIZ, S100A8, and S100A9, or any combination thereof. In some embodiments, a biopsy sample obtained from the cancer of the individual shows expression of at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, or at least 44 genes selected from CD4, CD8a, IL17A, IL17B, IL17C, IL17D, IL17F, IL17RA, IL17RC, C3, CCL2, CCL20, CSF2, CSF3, CXCL1, CXCL2, CXCL3, CXCL5, CXCL10, CXCR1, CXCR2, ICAM1, IL6, IL8, MMP1, MMP2, MMP3, MMP8, MMP9, MMP13, MMP14, MMP25, NCF4, NFKBIZ, S100A8, S100A9, SAA2, SAA1, SAA3, SAA4, TIMP1, TIMP2, TIMP3, and TIMP4. In certain embodiments, the IL-17 gene signature includes one or more genes selected from NFKBIZ, S100A8, and S100A9, or any combination thereof. For example, an individual may be tested for expression of IL-17 and/or an IL-17 gene signature in the cancer through a biopsy sample. If expression of IL-17 and/or an IL-17 gene signature is detected in the biopsy sample, the individual may be treated by any of the methods described herein.

Exemplary methods for determining expression of IL-17 and/or an IL-17 gene signature in a sample are described herein. For example, a biopsy sample may show expression of IL-17 or an IL-17 gene signature when the amount of IL-17 or an IL-17 gene signature in the biopsy sample is detectable by a particular assay, or when the amount of IL-17 or an IL-17 gene signature in the biopsy sample is detected above a threshold amount. For example, a threshold amount may include, without limitation: a raw threshold cycle (Ct) of less than 30 detected for one or more genes, as measured by q-PCR; a raw amount of gene expression (e.g., a threshold RPKM value) as determined by RNA-Seq; and a threshold amount of expression relative to one or more housekeeping genes (e.g., as described in the Examples below).

A biopsy sample from a cancer, such as a tumor biopsy sample, may contain multiple cell types. For example, a biopsy sample may contain tumor cells, various types of immune cells and other blood cells, tumor stroma, and so forth. IL-17 may be expressed in one or more of these cell types. As IL-17 is a secreted cytokine, once released from a cell associated with a tumor, IL-17 is able to interact with a number of different cell types expressing an IL-17 receptor. In some embodiments, IL-17 is expressed by T cells in the biopsy sample. In some embodiments, IL-17 is expressed by neutrophils in the biopsy sample. In some embodiments, IL-17 is expressed by macrophages in the biopsy sample. Further discussion of cell types that may express IL-17, and the role(s) of IL-17 in tumors, may be found, e.g., in Fontao, L., et al. Br. J. Dermatol. 166:687-9 (2012) and Chung, A. S., et al. Nat. Med. 19(9):1114-23 (2013). In some embodiments, a biopsy sample may be a formalin-fixed paraffin-embedded (FFPE) section of a tumor sample.

In some embodiments, one or more genes in an IL-17 gene expression signature are expressed by a cell that expresses IL-17. In some embodiments, one or more genes in an IL-17 gene expression signature are expressed by a cell that expresses an IL-17 receptor (e.g., a cell in which IL-17 signaling is activated by an interaction between IL-17 and an IL-17 receptor). In some embodiments, an IL-17 gene expression signature contains at least two genes, with one or more genes in the IL-17 gene expression signature expressed in a cell that expresses IL-17, and one or more genes in the IL-17 gene expression signature expressed in a cell that expresses an IL-17 receptor.

In some embodiments, expression of IL-17 may refer to expression of mRNA encoding IL-17. Various methods known in the art may be used to measure IL-17 mRNA in a biopsy sample, such as without limitation quantitative PCR (e.g., qRT-PCR or Taqman qPCR), in situ hybridization, Northern blotting, semi-quantitative PCR, RNA microarray, high-throughput RNA sequencing (e.g., RNA-Seq), NanoString assays (see, e.g., Geiss, G. K., et al. Nat. Biotechnol. 26(3):317-25 (2008)), and so forth. The level of IL-17 mRNA may be measured in absolute amount or normalized to the expression level of one or more control genes, such as housekeeping genes, rRNAs, etc., or the total amount of mRNA isolated from the biopsy sample.

In some embodiments, expression of IL-17 may refer to IL-17 protein expression. Various methods known in the art may be used to measure IL-17 protein in a biopsy sample, such as without limitation Western blotting, mass spectrometry, peptide microarray, immunoprecipitation, immunohistochemical staining, and so forth. The level of IL-17 protein may be measured in absolute amount or normalized to the expression level of one or more control proteins, such as housekeeping proteins, ribosomal proteins, etc., or the total amount of protein isolated from the biopsy sample.

In some embodiments, the biopsy sample obtained from the cancer shows elevated expression of IL-17 as compared to a reference sample. A “reference sample”, “reference cell”, “reference tissue”, “control sample”, “control cell”, or “control tissue”, as used herein, refers to a sample, cell, tissue, standard, or level that is used for comparison purposes. Any suitable reference sample known in the art may be used. For example, a reference sample may refer to a sample of the same tissue type as the biopsy sample taken from an individual without cancer. In other embodiments, a reference sample may refer to a sample of the same tumor type as the biopsy sample taken from an individual with a known or predicted responsiveness to treatment with a PD-1 axis binding antagonist as described herein. In another embodiment, a reference sample, reference cell, reference tissue, control sample, control cell, or control tissue is obtained from a healthy and/or non-diseased part of the body (e.g., tissue or cells) of the same subject or individual. For example, healthy and/or non-diseased cells or tissue adjacent to the diseased cells or tissue (e.g., cells or tissue adjacent to a tumor). In some embodiments, elevated expression of a gene or gene signature may refer to an absolute amount of expression. In some embodiments, elevated expression of a gene or gene signature may refer to an average, mean, or median expression level (e.g., an average/mean/median expression level across multiple different genes, or an average/mean/median expression level of one or more genes across multiple different samples).

In some embodiments, a biopsy sample obtained from the cancer of the individual shows expression of an IL-17 gene signature (e.g., one or more genes selected from IL-17A, IL-17F, IL-8, CSF3, CXCL1, CXCL3, CCL20, or any combination thereof). In some embodiments, a biopsy sample obtained from the cancer of the individual shows expression of an IL-17 gene signature (e.g., one or more genes selected from CD4, CD8a, IL17A, IL17B, IL17C, IL17D, IL17F, IL17RA, IL17RC, C3, CCL2, CCL20, CSF2, CSF3, CXCL1, CXCL2, CXCL3, CXCL5, CXCL10, CXCR1, CXCR2, ICAM1, IL6, IL8, MMP1, MMP2, MMP3, MMP8, MMP9, MMP13, MMP14, MMP25, NCF4, NFKBIZ, S100A8, S100A9, SAA2, SAA1, SAA3, SAA4, TIMP1, TIMP2, TIMP3, and TIMP4, or any combination thereof). In some embodiments, a biopsy sample obtained from the cancer of the individual shows expression of at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, or at least 44 genes selected from CD4, CD8a, IL17A, IL17B, IL17C, IL17D, IL17F, IL17RA, IL17RC, C3, CCL2, CCL20, CSF2, CSF3, CXCL1, CXCL2, CXCL3, CXCL5, CXCL10, CXCR1, CXCR2, ICAM1, IL6, IL8, MMP1, MMP2, MMP3, MMP8, MMP9, MMP13, MMP14, MMP25, NCF4, NFKBIZ, S100A8, S100A9, SAA2, SAA1, SAA3, SAA4, TIMP1, TIMP2, TIMP3, and TIMP4. In some embodiments, rather than detecting expression of an IL-17 mRNA or protein, expression of a gene signature reflective of or correlated with IL-17 signaling may be detected. An IL-17 gene signature may refer to a group of genes (or proteins) whose expression is thought or predicted to be functionally related or correlated with IL-17 signaling. For example, an IL-17 gene signature may include one or more genes whose expression may be regulated (positively or negatively) by IL-17 signaling, or it may include one or more genes whose expression may be correlated with IL-17 signaling. Such a gene signature may include expression of IL-17 (e.g., IL-17A and/or IL-17F) as well as IL-17 regulated or related genes (e.g., T cell markers CD4, CD8a; IL17 receptors IL17RA, IL17RC; and IL17 inducible genes C3, CCL2, CCL20, CSF2, CSF3, CXCL1, CXCL2, CXCL3, CXCL5, CXCL10, CXCR1, CXCR2, ICAM1, IL6, IL8, MMP1, MMP2, MMP3, MMP8, MMP9, MMP13, MMP14, MMP25, NCF4, NFKBIZ, S100A8, S100A9, SAA2, SAA1, SAA3, SAA4, TIMP1, TIMP2, TIMP3, and TIMP4; or any combination thereof). In certain embodiments, the IL-17 gene signature includes one or more genes selected from NFKBIZ, S100A8, and S100A9, or any combination thereof. In some embodiments, expression of an IL-17 gene signature may refer to mRNA expression, as described above. In some embodiments, expression of an IL-17 gene signature may refer to protein expression, as described above.

In some embodiments, the biopsy sample obtained from the cancer shows elevated expression of an IL-17 gene signature (e.g., one or more genes selected from IL-17A, IL-17F, IL-8, CSF3, CXCL1, CXCL3, CCL20, or any combination thereof). In some embodiments, the biopsy sample obtained from the cancer shows elevated expression of an IL-17 gene signature (e.g., one or more genes selected from CD4, CD8a, IL17A, IL17B, IL17C, IL17D, IL17F, IL17RA, IL17RC, C3, CCL2, CCL20, CSF2, CSF3, CXCL1, CXCL2, CXCL3, CXCL5, CXCL10, CXCR1, CXCR2, ICAM1, IL6, IL8, MMP1, MMP2, MMP3, MMP8, MMP9, MMP13, MMP14, MMP25, NCF4, NFKBIZ, S100A8, S100A9, SAA2, SAA1, SAA3, SAA4, TIMP1, TIMP2, TIMP3, and TIMP4, or any combination thereof). In some embodiments, the biopsy sample obtained from the cancer shows elevated expression of at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, or at least 44 genes selected from CD4, CD8a, IL17A, IL17B, IL17C, IL17D, IL17F, IL17RA, IL17RC, C3, CCL2, CCL20, CSF2, CSF3, CXCL1, CXCL2, CXCL3, CXCL5, CXCL10, CXCR1, CXCR2, ICAM1, IL6, IL8, MMP1, MMP2, MMP3, MMP8, MMP9, MMP13, MMP14, MMP25, NCF4, NFKBIZ, S100A8, S100A9, SAA2, SAA1, SAA3, SAA4, TIMP1, TIMP2, TIMP3, and TIMP4. In certain embodiments, the IL-17 gene signature includes one or more genes selected from NFKBIZ, S100A8, and S100A9, or any combination thereof. Detecting an IL-17 gene signature may involve measuring the expression of two or more individual genes (e.g., by mRNA or protein level) and deriving an average expression level for the signature as a whole. This average expression level may be optionally compared with a reference sample as described above. For example, the expression of one or more housekeeping genes, or the total amount of mRNA/protein in a reference sample, may be compared to the expression of an IL-17 gene signature measured in the biopsy sample.

The efficacy of any of the methods described herein (e.g., combination treatments including administering an effective amount of a combination of a PD-1 axis binding antagonist and an IL-17 binding antagonist) may be tested in various models known in the art, such as clinical or pre-clinical models. Suitable pre-clinical models may include without limitation ID8 ovarian cancer, GEM models, B16 melanoma, RENCA renal cell cancer, and Cloudman melanoma models of cancer.

The efficacy of any of the methods described herein (e.g., combination treatments including administering an effective amount of a combination of a PD-1 axis binding antagonist and an IL-17 binding antagonist) may be tested in an ID8 ovarian cancer model. For example, ID8 cells are injected into mice to develop tumors. Mice are randomly recruited into treatment groups receiving combination anti-PDL1 and anti-IL-17 treatment or control treatment. Tumor size (e.g., tumor volume) is measured during the course of treatment, and overall survival rate is also monitored. For further description of the ID8 model, see, e.g., Janat-Amsbury, M. M., et al. Anticancer Res. 26:3223-8 (2006).

The efficacy of any of the methods described herein (e.g., combination treatments including administering an effective amount of a combination of a PD-1 axis binding antagonist and an IL-17 binding antagonist) may be tested in a GEM model that develops tumors, including without limitation GEM models of non-small-cell lung cancer, pancreatic ductal adenocarcinoma, or melanoma. For example, a mouse expressing Kras^{G12D} in a p53^{null} background after adenoviral recombinase treatment as described in Jackson, E. L., et al. (2001) Genes Dev. 15(24):3243-8 (description of Kras^{G12D}) and Lee, C. L., et al. (2012) Dis. Model Mech. 5(3):397-402 (FRT-mediated p53^{null} allele) may be used as a pre-clinical model for non-small-cell lung cancer. As another example, a mouse expressing Kras^{G12D} in a p16/p19^{null} background as described in Jackson, E. L., et al. (2001) Genes Dev. 15(24):3243-8 (description of Kras^{G12D}) and Aguirre, A. J., et al. (2003) Genes Dev. 17(24):3112-26 (p16/p19^{null} allele) may be used as a pre-clinical model for pancreatic ductal adenocarcinoma (PDAC). As a further example, a mouse with melanocytes expressing Braf^{V600E} in a melanocyte-specific PTEN^{null}background after inducible (e.g., 4-OHT treatment) recombinase treatment as described in Dankort, D., et al. (2007) Genes Dev. 21(4):379-84 (description of Braf^{V600E}) and Trotman, L. C., et al. (2003) PLoS Biol. 1(3):E59 (PTEN^{null} allele) may be used as a pre-clinical model for melanoma. For any of these exemplary models, after developing tumors, mice are randomly recruited into treatment groups receiving combination anti-PDL1 and anti-IL-17 treatment or control treatment. Tumor size (e.g., tumor volume) is measured during the course of treatment, and overall survival rate is also monitored.

The efficacy of any of the methods described herein (e.g., combination treatments including administering an effective amount of a combination of a PD-1 axis binding antagonist and an IL-17 binding antagonist) may be tested in a mouse model for melanoma, such as the B16 cell-based subcutaneous melanoma model described in Overwijk, W. W. and Restifo, N. P. (2001) Curr. Protoc. Immunol. Chapter 20:Unit 20.1. After developing tumors, mice are randomly recruited into treatment groups receiving combination anti-PDL1 and anti-IL-17 treatment or control treatment. Tumor size (e.g., tumor volume) is measured during the course of treatment, and overall survival rate is also monitored.

The efficacy of any of the methods described herein (e.g., combination treatments including administering an effective amount of a combination of a PD-1 axis binding antagonist and an IL-17 binding antagonist) may be tested in a mouse model for renal cancer, such as the RENCA cell-based model described in Wiltrout, R. H., et al., pp. 13-19 in Immunotherapy of Renal Cell Carcinoma, Debruyne, F. M. J., et al., eds., Springer Berlin Heidelberg (1991). After developing tumors, mice are randomly recruited into treatment groups receiving combination anti-PDL1 and anti-IL-17 treatment or control treatment. Tumor size (e.g., tumor volume) is measured during the course of treatment, and overall survival rate is also monitored.

The efficacy of any of the methods described herein (e.g., combination treatments including administering an effective amount of a combination of a PD-1 axis binding antagonist and an IL-17 binding antagonist) may be tested in a mouse model for melanoma, such as the Cloudman cell-based model described in Nordlund, J. J. and Gershon, R. K. (1975). J. Immunol. 114(5):1486-90. After developing tumors, mice are randomly recruited into treatment groups receiving combination anti-PDL1 and anti-IL-17 treatment or control treatment. Tumor size (e.g., tumor volume) is measured during the course of treatment, and overall survival rate is also monitored.

The role of IL-17 in tumor progression may be tested in a mouse model with impaired or abrogated IL-17 signaling, e.g., to test the contribution of IL-17 signaling towards responsiveness to a PD-1 axis binding antagonist-based therapy (e.g., treatment with an anti-PDL1 antibody). For example, a mouse knockout lacking one or more IL-17 receptor genes may be used to model responsiveness to anti-PDL1 treatment, as compared to a mouse with normal IL-17 function. After being induced to develop tumors (e.g., by injecting with a tumor cell line, as described above), IL-17 receptor knockout and wild-type control mice receive anti-PDL1 or control treatment. Tumor size (e.g., tumor volume) is measured for all four conditions during the course of treatment, and overall survival rate is also monitored.

In another aspect, provided herein are methods for enhancing immune function in an individual having cancer comprising administering an effective amount of a combination of a PD-1 axis binding antagonist and an IL-17 binding antagonist.

In some embodiments of the methods of the present disclosure, the cancer has elevated levels of T cell infiltration. As used herein, T cell infiltration of a cancer may refer to the presence of T cells, such as tumor-infiltrating lymphocytes (TILs), within or otherwise associated with the cancer tissue. It is known in the art that T cell infiltration may be associated with improved clinical outcome in certain cancers (see, e.g., Zhang et al., N. Engl. J. Med. 348(3):203-213 (2003)).

However, T cell exhaustion is also a major immunological feature of cancer, with many tumor-infiltrating lymphocytes (TILs) expressing high levels of inhibitory co-receptors and lacking the capacity to produce effector cytokines (Wherry, E. J. Nature immunology 12: 492-499 (2011); Rabinovich, G. A., et al., Annual review of immunology 25:267-296 (2007)). In some embodiments of the methods of the present disclosure, the individual has a T cell dysfunctional disorder. In some embodiments of the methods of the present disclosure, the T cell dysfunctional disorder is characterized by T cell anergy or decreased ability to secrete cytokines, proliferate or execute cytolytic activity. In some embodiments of the methods of the present disclosure, the T cell dysfunctional disorder is characterized by T cell exhaustion. In some embodiments of the methods of the present disclosure, the T cells are CD4+ and CD8+ T cells.

In some embodiments of the methods of the present disclosure, activated CD4 and/or CD8 T cells in the individual are characterized by γ-IFN⁺ producing CD4 and/or CD8 T cells and/or enhanced cytolytic activity relative to prior to the administration of the combination. γ-IFN⁺ may be measured by any means known in the art, including, e.g., intracellular cytokine staining (ICS) involving cell fixation, permeabilization, and staining with an antibody against γ-IFN. Cytolytic activity may be measured by any means known in the art, e.g., using a cell killing assay with mixed effector and target cells.

In some embodiments of the methods of the present disclosure, CD4 and/or CD8 T cells exhibit increased release of cytokines selected from the group consisting of IFN-γ, TNF-α and interleukins. Cytokine release may be measured by any means known in the art, e.g., using Western blot, ELISA, or immunohistochemical assays to detect the presence of released cytokines in a sample containing CD4 and/or CD8 T cells.

In some embodiments of the methods of the present disclosure, the CD4 and/or CD8 T cells are effector memory T cells. In some embodiments of the methods of the present disclosure, the CD4 and/or CD8 effector memory T cells are characterized by having the expression of CD44^{high} CD62L^{low}. Expression of CD44^{high} CD62L^{low} may be detected by any means known in the art, e.g., by preparing single cell suspensions of tissue (e.g., a cancer tissue) and performing surface staining and flow cytometry using commercial antibodies against CD44 and CD62L.

Any of the PD-1 axis binding antagonists and the IL-17 binding antagonists known in the art or described herein may be used in the methods of the present disclosure.

### VI. Kits or Articles of Manufacture

In another aspect, provided herein are kits or articles of manufacture comprising a package insert and a PD-1 axis binding antagonist and/or an IL-17 binding antagonist. Such kits or articles of manufacture may be used to treat or delay progression of cancer in an individual and/or to enhance immune function in an individual having cancer. In some embodiments, the package insert comprises instructions for using the kit or article of manufacture.

In some embodiments, provided herein are kits comprising a PD-1 axis binding antagonist and a package insert comprising instructions for using the PD-1 axis binding antagonist in combination with an IL-17 binding antagonist to treat or delay progression of cancer in an individual. In some embodiments, provided herein are kits comprising a PD-1 axis binding antagonist and an IL-17 binding antagonist, and a package insert comprising instructions for using the PD-1 axis binding antagonist and the IL-17 binding antagonist to treat or delay progression of cancer in an individual. In some embodiments, provided herein are kits comprising an IL-17 binding antagonist and a package insert comprising instructions for using the IL-17 binding antagonist in combination with a PD-1 axis binding antagonist to treat or delay progression of cancer in an individual. In some embodiments, provided herein are kits comprising a PD-1 axis binding antagonist and a package insert comprising instructions for using the PD-1 axis binding antagonist in combination with an IL-17 binding antagonist to enhance immune function in an individual having cancer. In some embodiments, provided herein are kits comprising a PD-1 axis binding antagonist and an IL-17 binding antagonist, and a package insert comprising instructions for using the PD-1 axis binding antagonist and the IL-17 binding antagonist to enhance immune function in an individual having cancer. In some embodiments, provided herein are kits comprising an IL-17 binding antagonist and a package insert comprising instructions for using the IL-17 binding antagonist in combination with a PD-1 axis binding antagonist to enhance immune function in an individual having cancer.

In some embodiments, the PD-1 axis binding antagonist and the IL-17 binding antagonist are in the same container or separate containers. Suitable containers include, for example, bottles, vials, bags and syringes. The container may be formed from a variety of materials such as glass, plastic (such as polyvinyl chloride or polyolefin), or metal alloy (such as stainless steel or hastelloy). In some embodiments, the container holds the formulation and the label on, or associated with, the container may indicate directions for use. The article of manufacture or kit may further include other materials desirable from a commercial and user standpoint, including other buffers, diluents, filters, needles, syringes, and package inserts with instructions for use. In some embodiments, the article of manufacture further includes one or more of another agent (e.g., a chemotherapeutic agent, and anti-neoplastic agent). Suitable containers for the one or more agent include, for example, bottles, vials, bags and syringes.

In some embodiments, the kit comprises a container containing one or more of the PD-1 axis binding antagonists and IL-17 binding antagonists described herein. Suitable containers include, for example, bottles, vials (e.g., dual chamber vials), syringes (such as single or dual chamber syringes) and test tubes. The container may be formed from a variety of materials such as glass or plastic. In some embodiments, the kit may comprise a label (e.g., on or associated with the container) or a package insert. The label or the package insert may indicate that the compound contained therein may be useful or intended for treating or delaying progression of cancer in an individual or for enhancing immune function of an individual having cancer. The kit may further comprise other materials desirable from a commercial and user standpoint, including other buffers, diluents, filters, needles, and syringes.

The specification is considered to be sufficient to enable one skilled in the art to practice the invention. Various modifications of the invention in addition to those shown and described herein will become apparent to those skilled in the art from the foregoing description and fall within the scope of the appended claims. All publications, patents, and patent applications cited herein are hereby incorporated by reference in their entirety for all purposes.

### EXAMPLES

The invention can be further understood by reference to the following examples, which are provided by way of illustration and are not meant to be limiting.

### Example 1: Testing Efficacy of Combination Treatments Using Anti-PDL1 and Anti-IL-17 in an EMT6 Breast Carcinoma Model

Many factors may contribute to the overall effectiveness of cancer treatments, particularly those targeting anti-tumor immunity. It has been observed in particular patient cohorts that the expression of IL-17F correlates with non-responsiveness or a late response to anti-PDL1 treatment. Therefore, the efficacy of combination treatments including anti-PDL1 and anti-IL-17 was tested in an EMT6 syngeneic tumor model.

90 BALB/c mice were inoculated subcutaneously in the left 4th mammary fat pad with 0.1 million EMT6 cells in 100 microliters of HBSS+matrigel (BD Biosciences). Mice were allowed to grow tumors. When tumors achieved a mean tumor volume of approximately 150 mm3 (Day 0, approximately 10 days after inoculation), animals were recruited into treatment groups outlined below (extra animals were euthanized).

Treatment was initiated on Day 1. The first dose was given intravenously (IV), and the remaining doses were administered via intraperitoneal injection (IP) for 3 times/week for 3 weeks (i.e., 10 total doses, of which 1 is IV and the remaining 9 are IP), as described below. Dosing began on day 1 for gp120 control and anti-IL-17 treatments. Dose volume was 100 μL.

Mice were divided into 4 treatment groups receiving the following doses: (1) Mouse IgG1 anti-gp120 9338 (20 mg/kg) and mouse IgG2a (10 mg/kg) dosed IV for first dose, then dosed IP 3×/week for 3 weeks; (2) Anti-IL17 cross-reactive antibody recognizing IL-17A and IL-17F (10 mg/kg) and Anti-IL17 antibody recognizing IL-17F (10 mg/kg) dosed IV for first dose, then dosed IP 3×/week for 3 weeks; (3) anti-PDL1 (10 mg/kg) dosed IV for first dose, then dosed IP 3×/week for 3 weeks; and (4) anti-PDL1 (10 mg/kg), Anti-IL17 cross-reactive antibody recognizing IL-17A and IL-17F (10 mg/kg), and Anti-IL17 antibody recognizing IL-17F (10 mg/kg) dosed IV for first dose, then dosed IP 3×/week for 3 weeks.

Measurements and weights were collected twice per week. Animals exhibiting weight loss of >15% were weighed daily and euthanized if they lost >20% body weight. Animals showing adverse clinical issues were observed more frequently, up to daily depending on severity, and euthanized if moribund. Mice were euthanized if tumor volumes exceeded 3,000 mm3, or after 3 months if tumors did not form. Previous studies have shown that after 8 weeks, remaining tumors have a reduced growth rate and are significantly less aggressive. These remaining tumors were measured and weighed once per week. For any large or aggressively growing tumors present after 8 weeks, measurements and weights for these specific mice were collected twice per week. Throughout the entire study, clinical observations of all mice were performed twice per week.

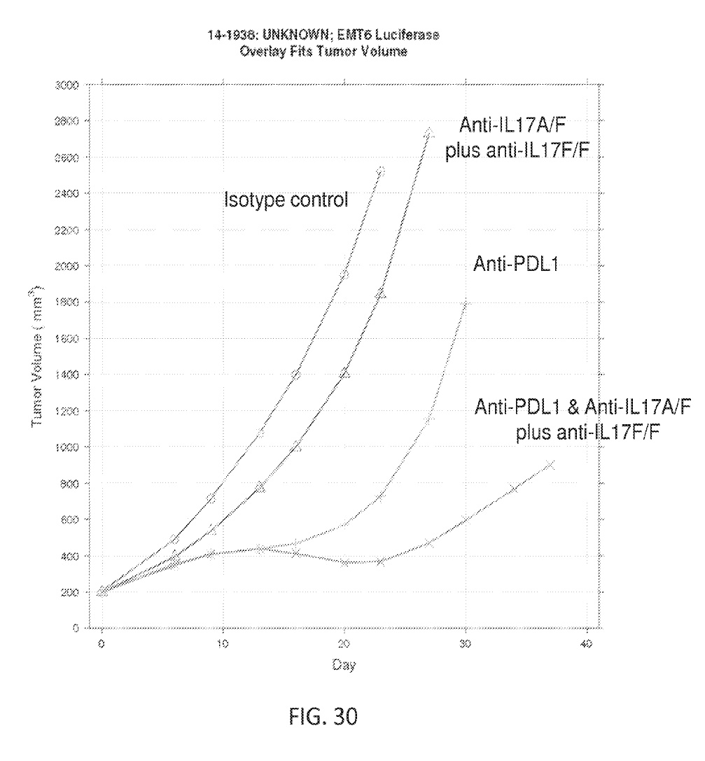

As shown in FIG. 30, treatment with IL-17 antibodies alone resulted in a slight decrease in tumor size, as compared to treatment with control antibody. Treatment with anti-PDL1 resulted in a slightly larger decrease in tumor size, as compared to treatment with anti-IL-17 antibodies. However, combination treatment with anti-PDL1 antibody and anti-IL-17 antibodies resulted in a dramatic decrease in tumor size and tumor growth rate, as compared to either single treatment or control treatment. These results indicate that combination treatments including both a PD-1 axis binding antagonist (e.g., anti-PDL1) and an IL-17 binding antagonist (e.g., anti-IL-17) have superior efficacy in treating cancer, particularly in comparison with control treatment and/or each treatment alone.

### Example 2: Analysis of IL-17 Expression and Disease Progression During Anti-PDL1 Treatment

The previous Example demonstrates that combination treatments including both a PD-1 axis binding antagonist and an IL-17 binding antagonist show superior efficacy in treating cancer as compared to each treatment alone. Therefore, it is of interest to determine whether IL-17 expression may serve as a biomarker for selecting patients for a treatment with a PD-1 axis binding antagonist and an IL-17 binding antagonist. This Example analyzes the correlation between expression of IL-17 family cytokines and response to anti-PDL1 treatment for multiple cancer types.

Materials and Methods

### Gene Expression Analyses

Hematoxylin-eosin (H&E) sections were prepared for all samples and were reviewed by a pathologist to confirm diagnosis and assess tumor content. RNA extraction and gene expression analysis was performed as described in Schleifman and colleagues (Schleifman E B, et al., (2014) PloS one 9(2):e88401). Briefly, FFPE sections were macrodissected to enrich for neoplastic tissue followed by RNA extraction using the High Pure FFPE RNA Micro Kit (Roche Applied Sciences, Indianapolis, Ind.). RNA was then subjected to a one-step cDNA synthesis/preamplification reaction using the Invitrogen Platinum Taq/Reverse Transcriptase enzyme mix and pooled TaqMan® Gene Expression Assays (Life Technologies, Carlsbad, Calif.). Quantitative PCR (qPCR) was then conducted on Fluidigm 96.96 Dynamic Arrays using the BioMark™ HD system (Fluidigm Corporation, South San Francisco, Calif.). Cycle threshold (Ct) values were normalized. NanoString analyses for gene expression are conducted as described in Geiss, G. K., et al. Nat. Biotechnol. 26(3):317-25 (2008).

IHCIC refers to immunohistochemical staining specific to PDL1 expression in immune cells. Samples were stained for PDL1 expression and graded based on the percentage of tumor area with positive staining. The grading metric for evaluating staining depends on the tumor type. For example, for a non-small-cell lung cancer sample, a grade of IHCIC 2+ refers to a sample in which greater than 5% but less than 10% of the tumor area is occupied by PDL1-positive stained cells, and a grade of IHCIC 3+ refers to a sample in which greater than 10% of the tumor area is occupied by PDL1-positive stained cells.

### Anti-PDL1 Treatment and RECIST Response

All patients were treated with ≧1-20 mg/kg of anti-PDL1 with a baseline tumor assessment. Objective response rate was assessed by RECIST v1.1. For melanoma, renal cell carcinoma, and non-small-cell lung cancer patients, confirmed response (BOCR) was used. For bladder cancer patients, unconfirmed response (BURSP) was used.

For association of gene expression signatures with RECIST response, the p-value was derived from a logistic regression model with response as the outcome and continuous gene expression as the independent variable. Model was adjusted for IHCIC (categorical) and IHCTC, or immunohistochemical staining in tumor cells (≧50%; categorical). Black: IHCIC0; orange: IHCIC1; magenta: IHCIC2; red: IHCIC3. Triangle symbols depict IHC TC≧50%.

### ROC Analyses

ROC curves were generated by plotting sensitivity vs 1-specificity at varying biomarker cutoffs. In the comparison of patients with complete or partial response to patients with stable or progressive disease, sensitivity was defined as the percentage of patients with complete or partial response which were correctly identified as such, and specificity is defined as the percentage of patients with stable or progressive disease which were correctly identified as such. In the comparison of patients with complete response, partial response, or stable disease to patients with progressive disease, sensitivity was defined as the percentage of patients with complete or partial response or stable disease which were correctly identified as such, and specificity is defined as the percentage of patients with progressive disease which were correctly identified as such.

### Analysis of IL-17 and Teff Gene Expression Signatures

Normalized cycle threshold (Ct) values were converted to relative expression values (negative delta Ct) by subtracting the median gene expression estimated using all 96 genes on the array. Th17 signature was generated by combining the expression of IL-17A, IL-17F, and RORC and the Teff signature contains expression of CD8, IFNgamma, granzyme A, granzyme B and peforin.

### Immunohistochemical Staining

Immunohistochemical staining was performed on formalin-fixed, paraffin-embedded, vendor-acquired tumor samples of non-small-cell lung cancer (n=13) and colorectal cancer (n=12) tissues. Anti-human IL-17A affinity purified goat polyclonal antibody was used for detection (R&D Systems Catalog No. AF-317) at 3 μg/mL according to standard immunohistochemical staining methods.

Results

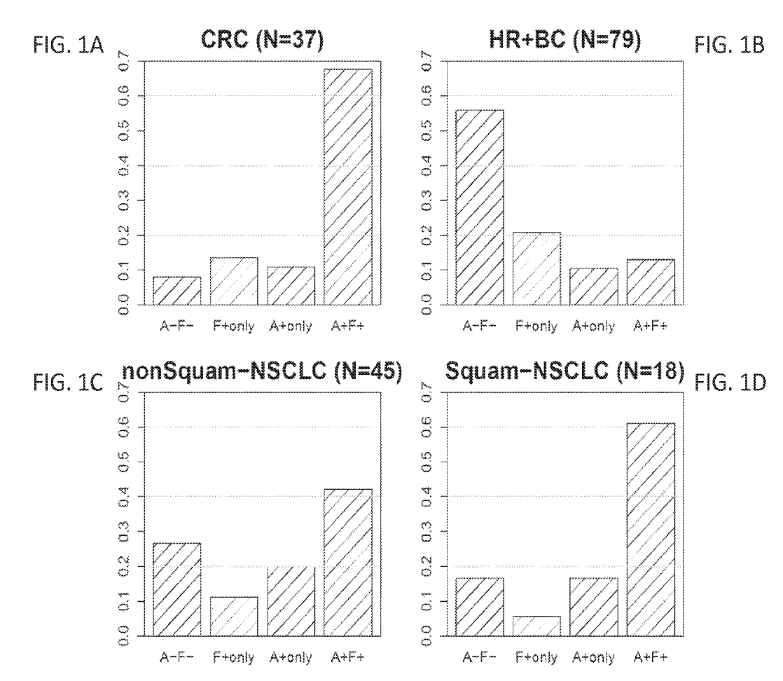

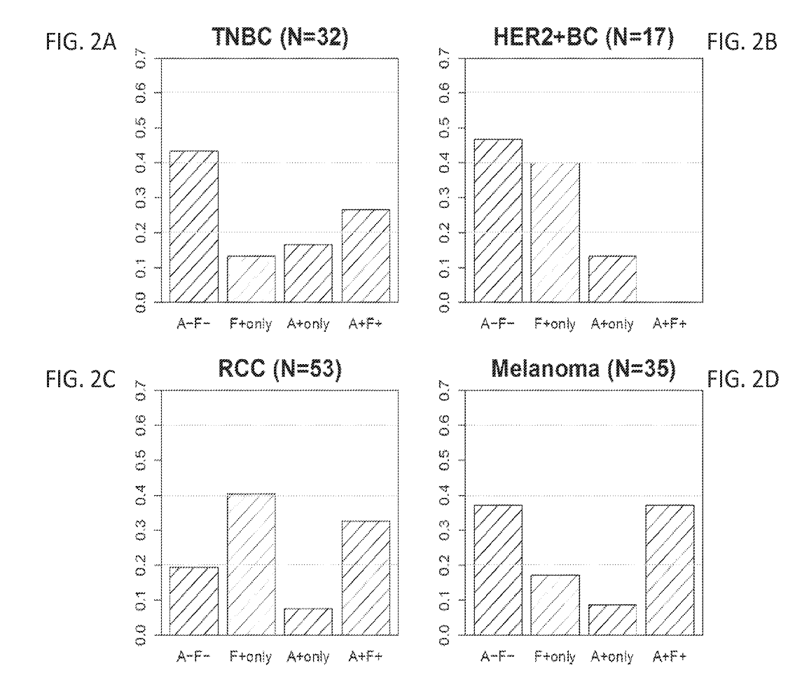

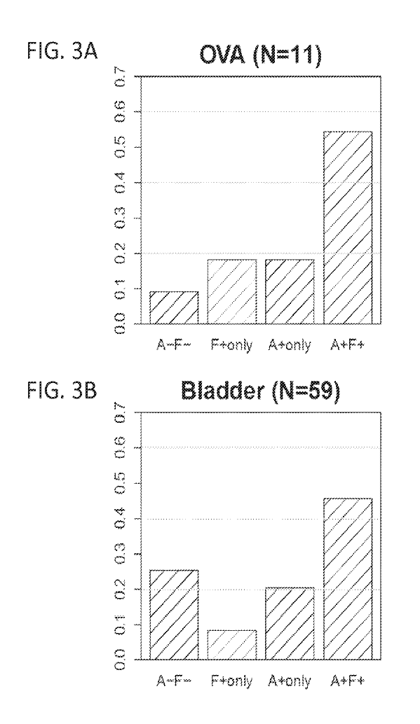

In order to study the potential contribution of IL-17 cytokine family members to anti-tumor immunity, the expression of IL-17A and IL-17F was measured in tumor tissues from various cancer types. FIGS. 1A-3B illustrate the prevalence of these two cytokines in various tumor tissues. Samples from each type of cancer tissue were categorized as showing no IL-17A or IL-17F expression, IL-17F expression only, IL-17A expression only, or expression of both IL-17A and IL-17F. A sample was deemed to show expression if that gene yielded a raw threshold cycle (Ct) of less than 30. Types of cancer tissues studied included: colorectal cancer (FIG. 1A), hormone receptor-positive breast cancer (FIG. 1B), non-squamous non-small-cell lung cancer (FIG. 1C), squamous non-small-cell lung cancer (FIG. 1D), triple negative breast cancer (FIG. 2A), HER2-positive breast cancer (FIG. 2B), renal cell carcinoma (FIG. 2C), melanoma (FIG. 2D), ovarian cancer (FIG. 3A), and bladder cancer (FIG. 3B).

As shown in FIGS. 1A-3B, the presence of IL-17A and IL-17F in cancers varied considerably between cancers. For example, greater than 60% of squamous non-small-cell lung cancer and colorectal cancer tissues showed both IL-17A and IL-17F expression, compared to none of the HER2-positive breast cancer tissues tested. Non-squamous non-small-cell lung cancer, ovarian cancer, bladder cancer, renal cell carcinoma, melanoma, and triple negative breast cancer also showed prevalence of IL-17A and IL-17F expression in the approximate range of 25-55%. These results demonstrate that IL-17 family cytokine expression is variable between cancers but is present in many distinct tumor types.

To determine whether IL-17 expression correlates with response to anti-PDL1 treatment, the expression of IL-17A and IL-17F was measured in cohorts of patients receiving anti-PDL1 treatment for different cancer types. Samples from each cohort were tested for a variety of factors: association between IL-17A/F (as used herein, IL-17A/F may collectively refer to analysis of IL-17A, IL-17F, IL-17A and IL-17F, and a lack of IL-17A and IL-17F) expression and response to treatment, analysis of IL-17A/F and IL-8 gene expression signatures, correlation between IL-17A/F and IL-8 expression and responsiveness to anti-PDL1 treatment in both total patients and patients IHCIC 2+ grade cancers, association of RECIST response and IL-17 gene signature, and ROC analysis of IL-17 gene signature in patients with complete or partial response to anti-PDL1 treatment compared to patients with stable or progressive disease with anti-PDL1 treatment (or an ROC analysis of IL-17 gene signature in patients with complete response, partial response, or stable disease with anti-PDL1 treatment compared to patients with progressive disease with anti-PDL1 treatment). Each analysis was performed using melanoma, renal cell carcinoma, bladder cancer, and non-small-cell lung cancer patient cohorts.

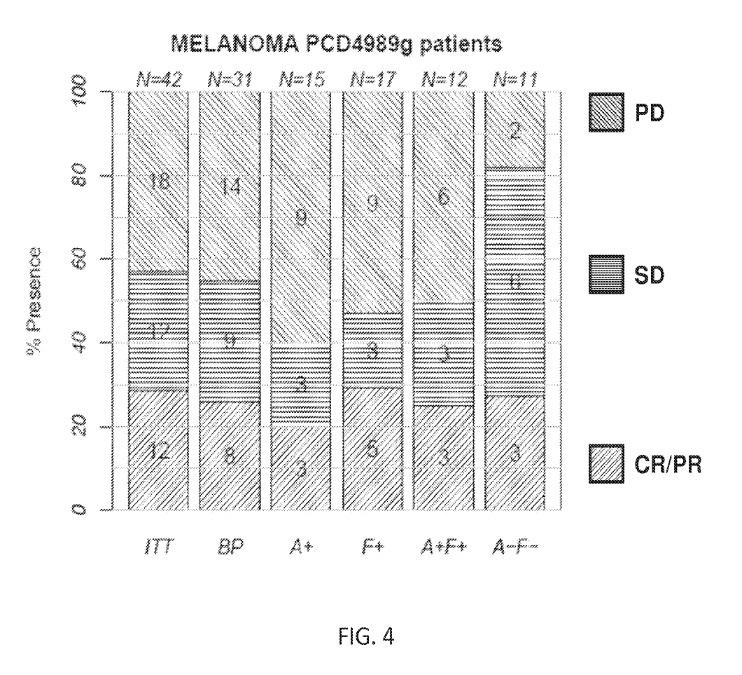

FIG. 4 reports the presence of IL-17A/F gene expression in patients showing a complete or partial response to anti-PDL1 treatment, stable disease with anti-PDL1 treatment, and progressive disease with anti-PDL1 treatment. As shown, the patients showing an absence of both IL-17A and IL-17F expression trend towards an increased clinical benefit (i.e., a complete or partial response or stable disease) of anti-PDL1 treatment.

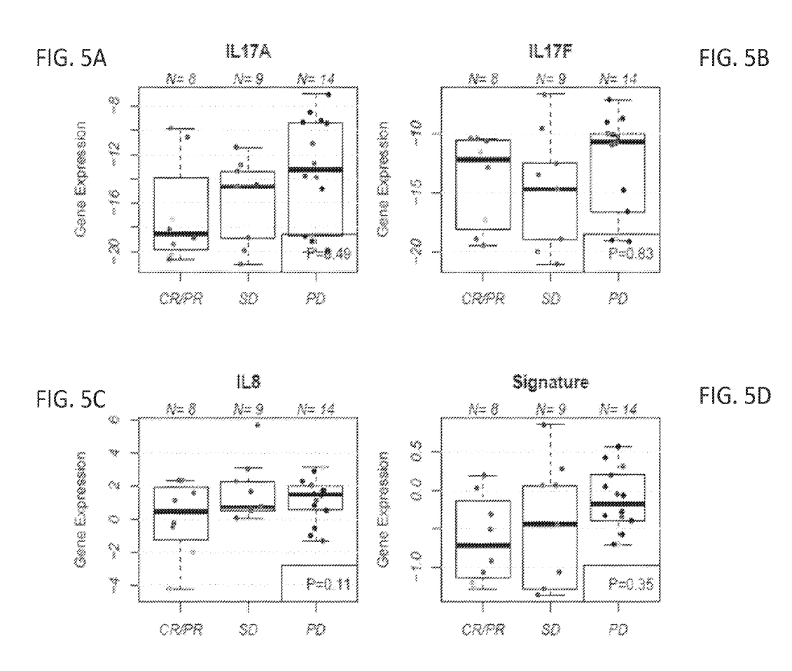

As shown in FIGS. 5A-5D, the association between IL-17A (FIG. 5A) and IL-17F (FIG. 5B) expression and RECIST response to anti-PDL1 treatment was analyzed. The association between RECIST response to anti-PDL1 treatment and an IL-17-induced gene, IL-8, was also analyzed (FIG. 5C). Finally, the association between an IL-17 gene expression signature and RECIST response to anti-PDL1 treatment was analyzed (FIG. 5D). This IL-17 gene expression signature was derived from the average gene expression level of all three genes (IL-17A, IL-17F, and IL-8). When all patients were analyzed, a general trend emerged of greater prevalence of IL-17 expression and IL-17 gene signature in progressive disease patients.

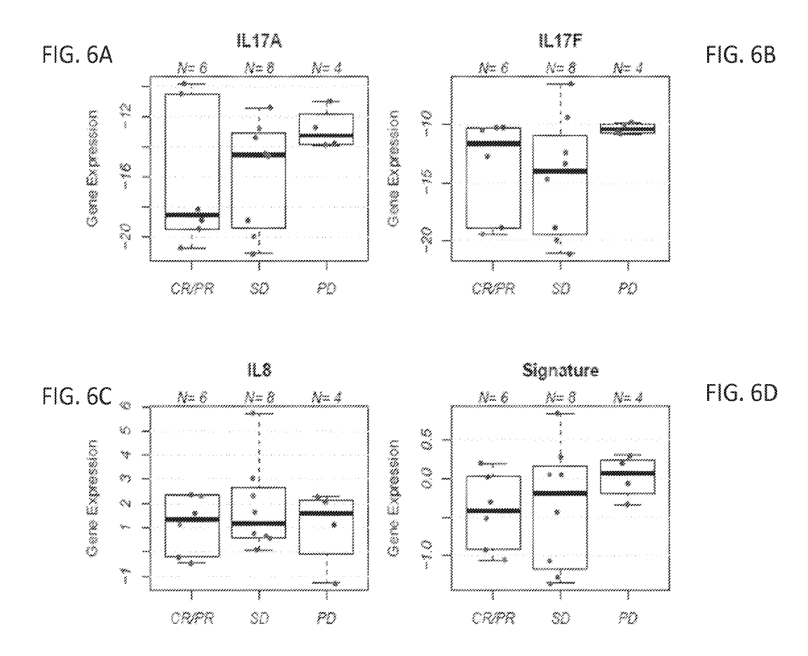

FIGS. 6A-6D show that this association between progressive disease and IL-17 expression was much clearer when only patients with IHCIC 2+ patients were analyzed. This was particularly true for IL-17A (FIG. 6A) and IL-17F (FIG. 6B) expression.

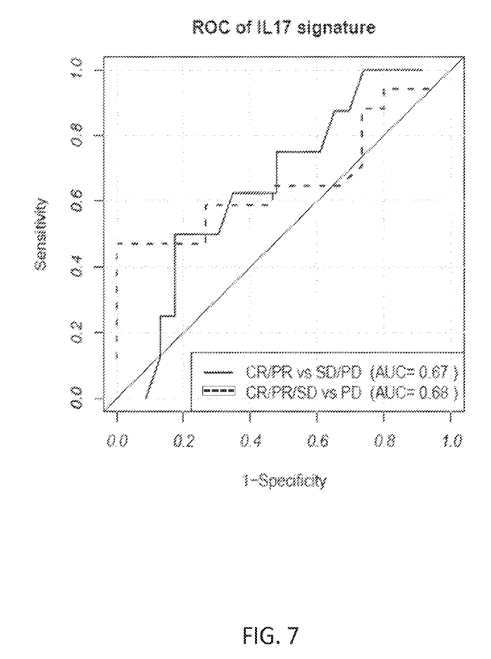

ROC analyses also predicted a clinical benefit for IL-17 gene expression signature (as described above) as an indicator of response to anti-PDL1 treatment (FIG. 7). ROC analysis was performed by comparing patients exhibiting a complete response or partial response with PDL1 treatment to patients exhibiting a stable disease or progressive disease with PDL1 treatment. ROC analysis was also performed by comparing patients exhibiting a complete response, partial response, or stable disease with PDL1 treatment to patients exhibiting progressive disease with PDL1 treatment. In both cases, the AUC values were above 0.5, indicating a clinical benefit for IL-17 gene expression signature.

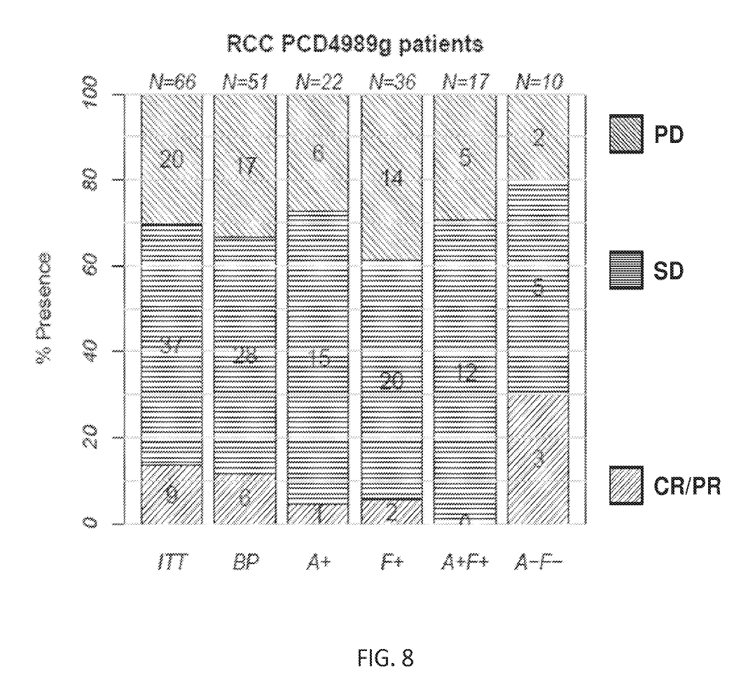

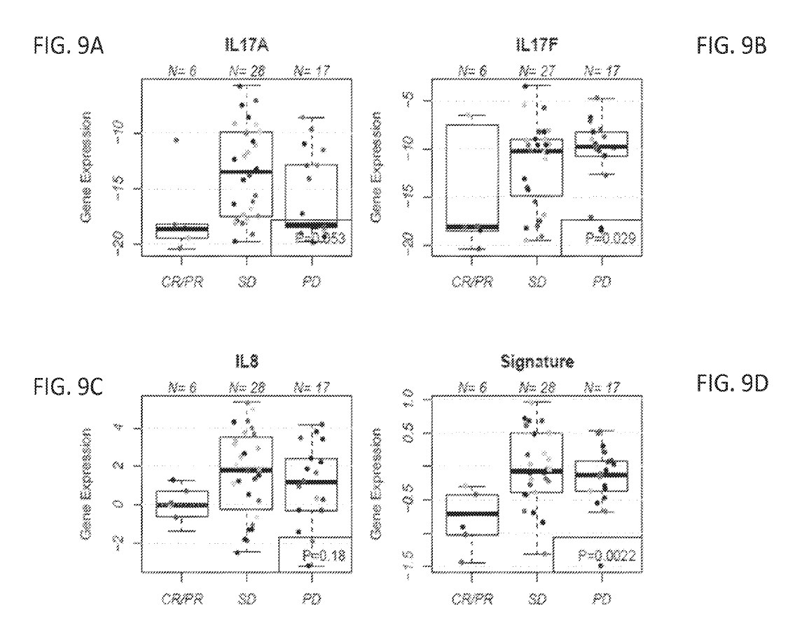

These analyses were repeated for renal cell carcinoma patients. FIG. 8 shows a trend toward higher responsiveness in patients negative for IL-17A and IL-17F expression. FIGS. 9A-9D show that higher IL-17 expression was significantly correlated in patients with stable or progressive disease on anti-PDL1 treatment, particularly for IL-17F expression (FIG. 9B). In addition, analysis of the IL-17 gene expression signature showed an extremely statistically significant correlation with a stable or progressive disease response to anti-PDL1 treatment (FIG. 9D).

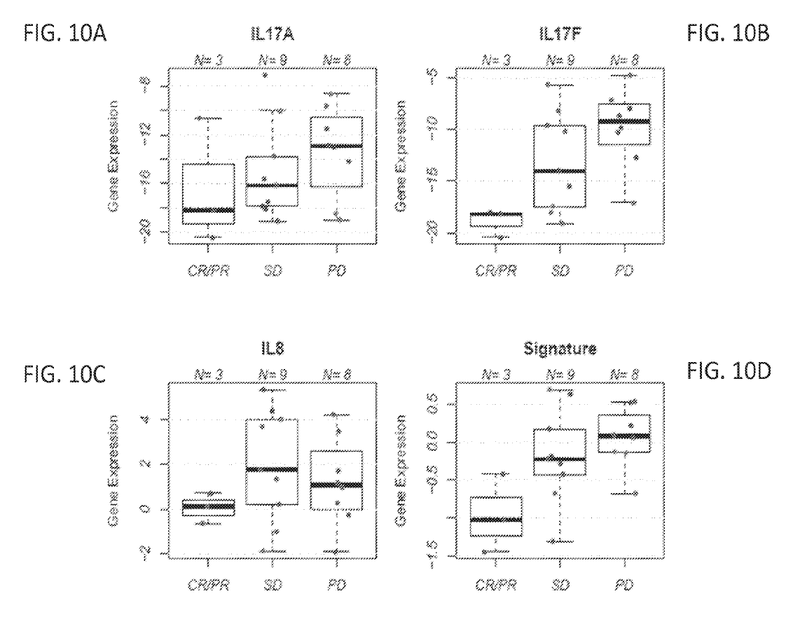

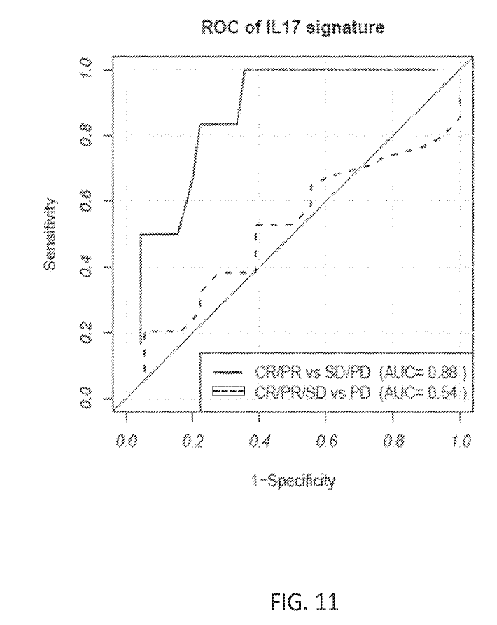

FIGS. 10A-10D show a trend toward higher IL-17A and IL-17F expression among IHCIC 2+ patients with progressive disease on anti-PDL1 treatment. ROC analysis of responsive vs. non-responsive patients also predicted a clinical benefit for IL-17 gene expression signature as an indicator of response to anti-PDL1 treatment (FIG. 11), particularly when comparing patients having a complete or partial response to patients with stable or progressive disease (AUC=0.88).

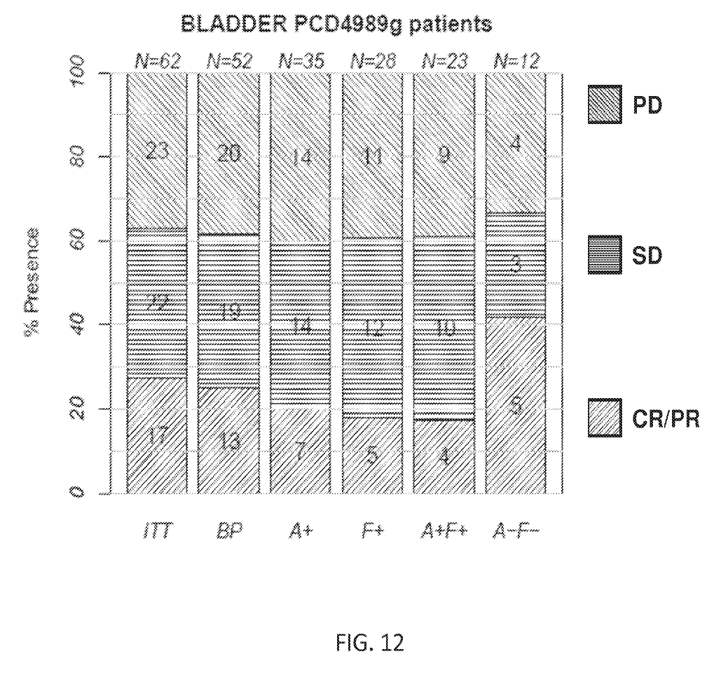

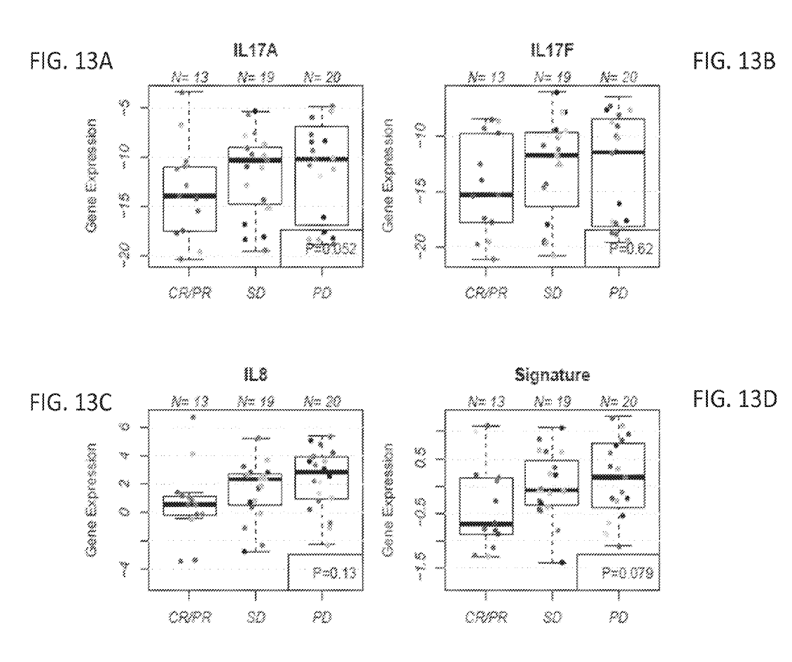

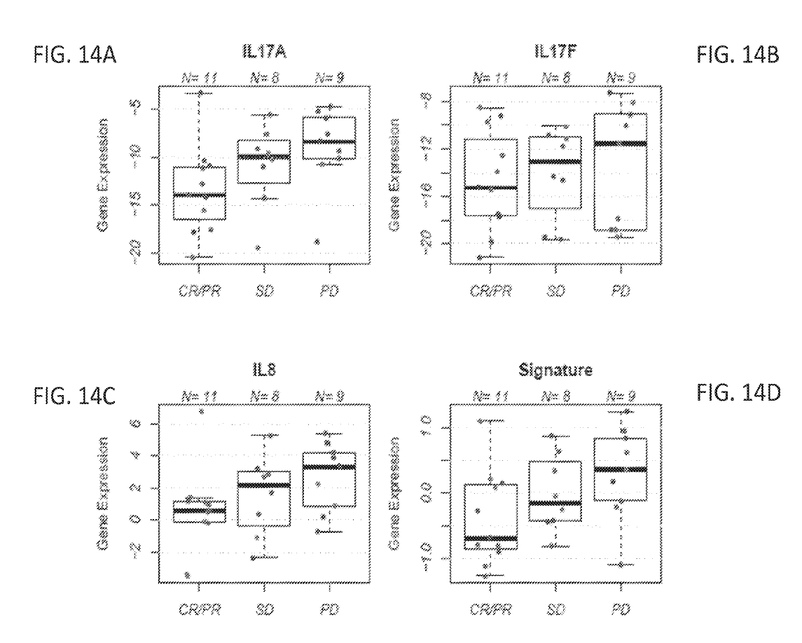

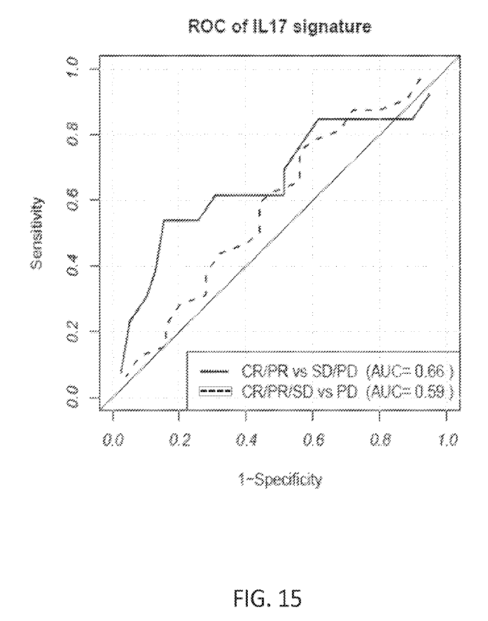

These analyses were repeated for bladder cancer patients. FIG. 12 shows a higher relative prevalence of complete or partial responses to anti-PDL1 treatment in patients negative for IL-17A and IL-17F expression. FIGS. 13A-13D show a general trend toward higher IL-17A, IL-17F, IL-8, and IL-17 gene signature expression in patients with stable or progressive disease on anti-PDL1 treatment. FIGS. 14A-14D confirm this trend in IHCIC 2+ samples. ROC analyses also predicted a clinical benefit for IL-17 gene expression signature as an indicator of response to anti-PDL1 treatment (FIG. 15).

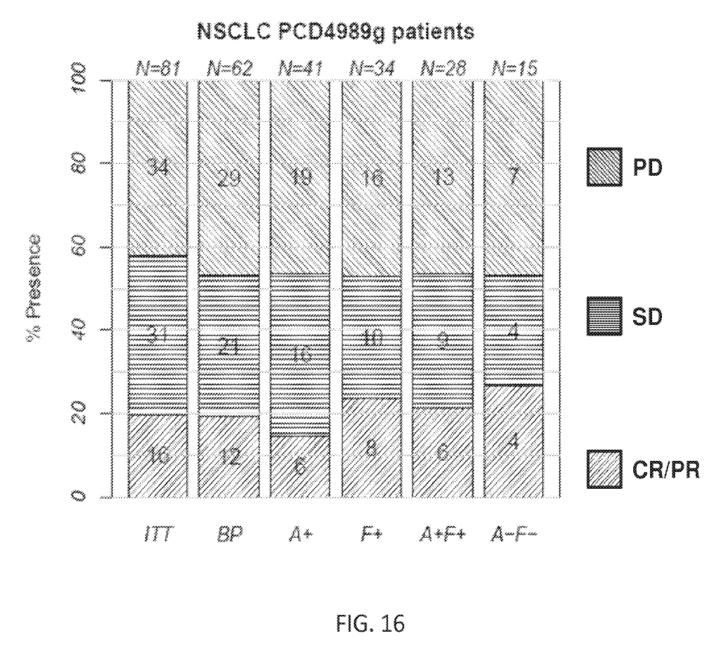

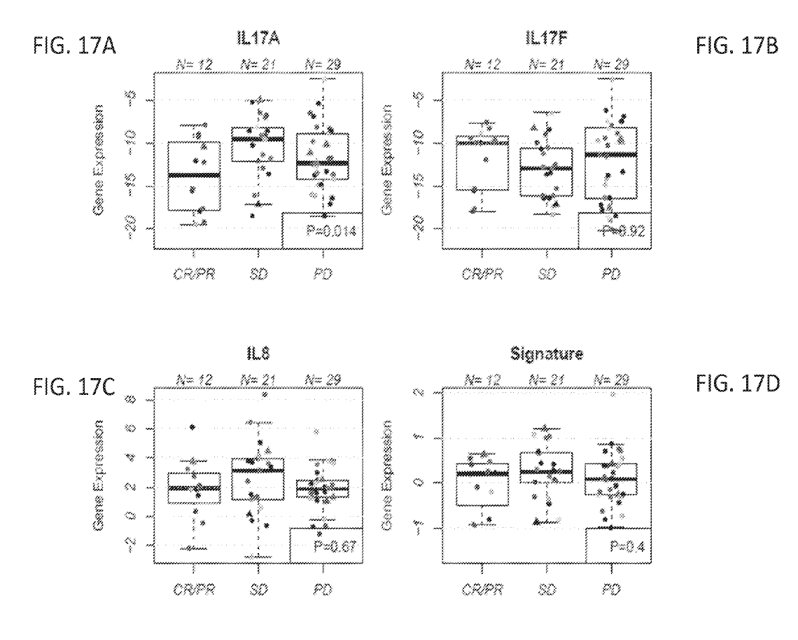

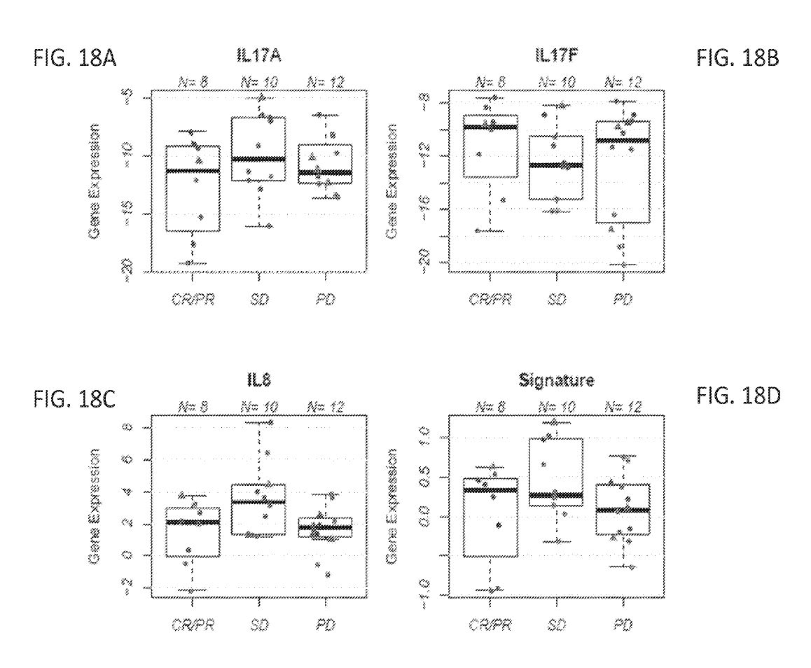

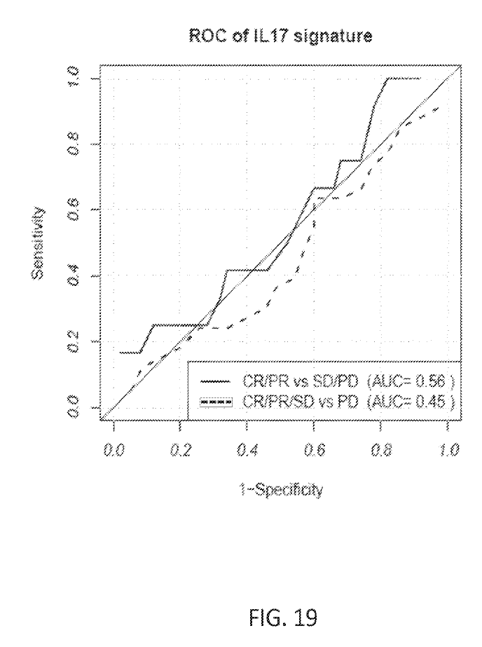

These analyses were repeated for non-small cell lung cancer patients. FIG. 16 shows that IL-17A/F expression was not observed to trend with response to anti-PDL1 treatment. Similarly, FIGS. 17A-17D and 18A-18D did not show a clear association between responsiveness to anti-PDL1 treatment and IL-17A/F expression. Finally, ROC analyses did not predict a clear clinical benefit for IL-17 gene expression signature as an indicator of response to anti-PDL1 treatment, with AUC values of 0.45 and 0.56 (FIG. 19).

Taken together, these results demonstrate that IL-17A/F expression may be useful as a predictor of non-responsiveness to anti-PDL1 treatment. Specifically, in many cancers (such as melanoma, bladder cancer, and renal cell carcinoma), a lack of IL-17A and IL-17F expression in tumors may predict a more positive clinical benefit of anti-PDL1 treatment than for tumors displaying expression of IL-17A/F.

To confirm these results using a different method and further investigate the effect of IL-17A/F expression on anti-PDL1 treatment in non-small-cell lung cancer, IL-17 expression was also examined using NanoString analysis. These analyses included a more extensive IL-17 gene expression signature that incorporated the expression of IL-17A and IL-17F, along with IL-17 induced genes IL-8, CSF3, CXCL1, CXCL3, and CCL20.

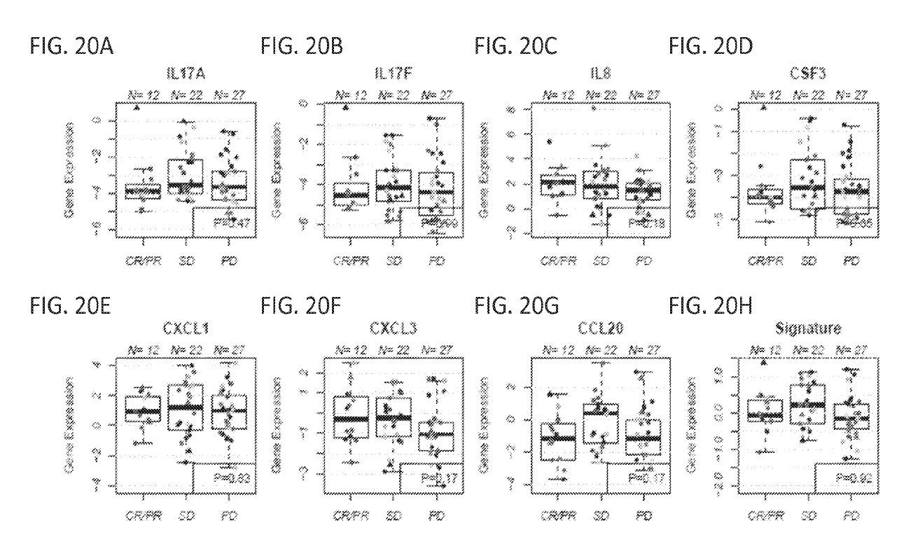

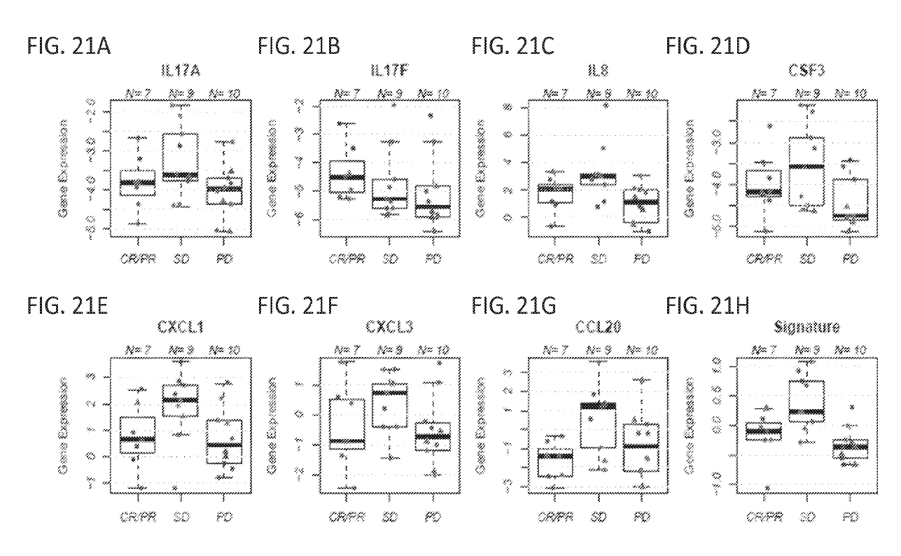

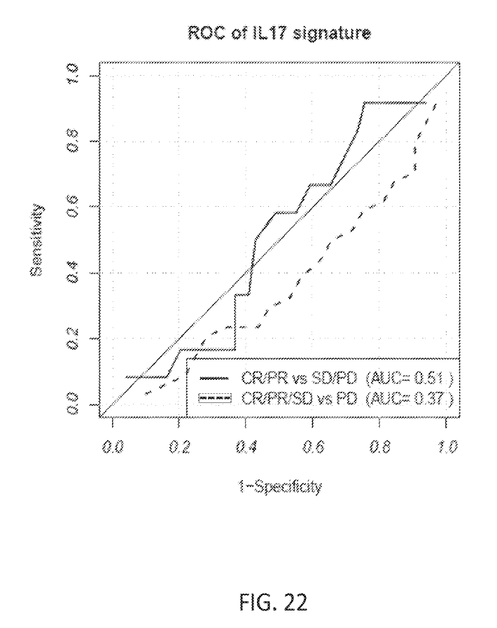

FIGS. 20A-20H show the correlations between IL-17A (FIG. 20A), IL-17F (FIG. 20B), IL-8 (FIG. 20C), CSF3 (FIG. 20D), CXCL1 (FIG. 20E), CXCL3 (FIG. 20F), and CCL20 (FIG. 20G) expression and responsiveness to anti-PDL1 treatment, as well as the correlation between the average of the entire IL-17 gene signature and responsiveness to anti-PDL1 treatment (FIG. 20H). FIGS. 21A-21H repeat these analyses on IHCIC 2+ patients. As shown in FIGS. 20A-20H and 21A-21H, no clear evidence of a correlation between IL-17 gene expression and responsiveness to anti-PDL1 treatment was observed. This lack of correlation in the data was confirmed by the lack of clinical benefit for IL-17 gene expression in anti-PDL1 treatment, as predicted by ROC analysis (FIG. 22). These NanoString results confirm a lack of correlation between IL-17 gene expression and anti-PDL1 response for non-small-cell lung cancer, a finding that is in agreement with the previous results but incorporates a more extensive IL-17 gene expression signature for analysis.

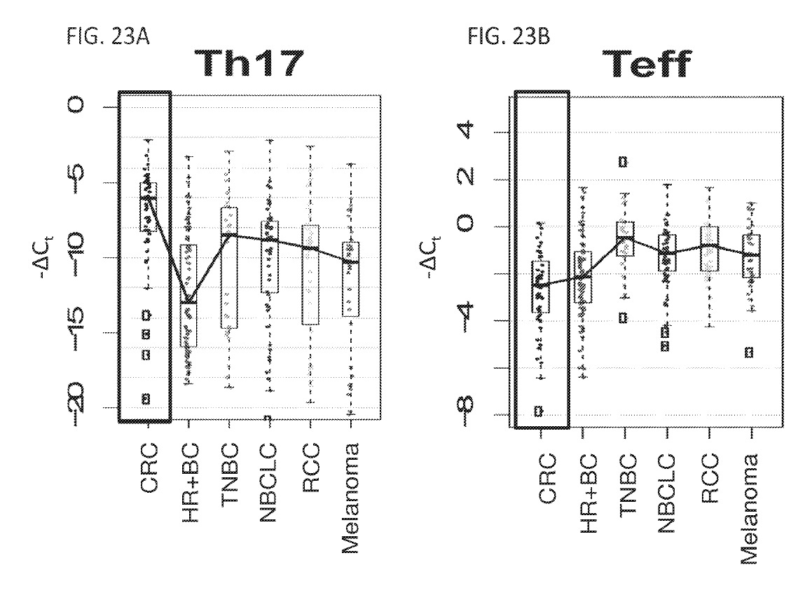

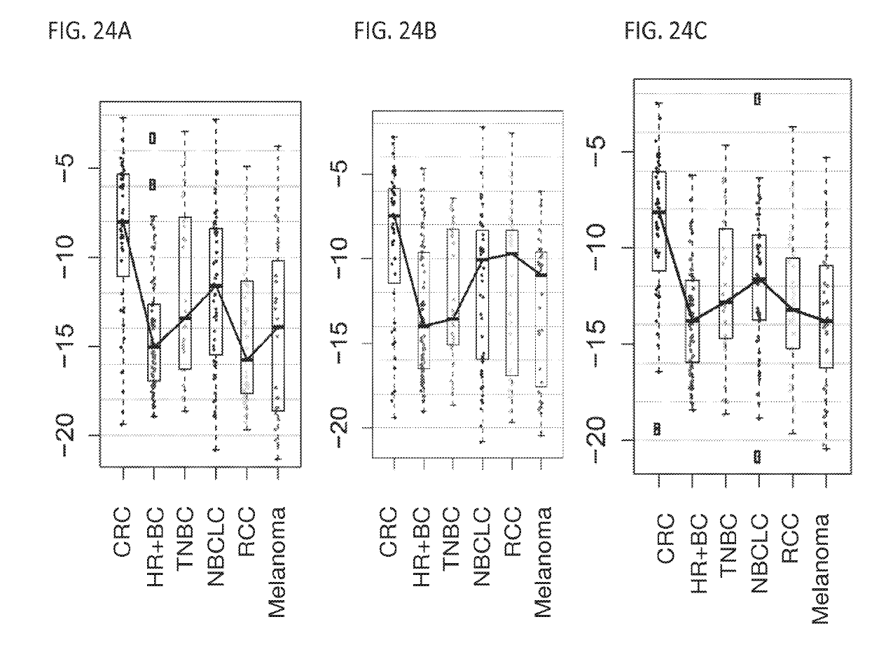

IL-17 and T_{effector} (Teff) gene signatures were also measured in various types of cancer. These results demonstrated a clear increase in IL-17 gene signature and decrease in Teff gene signature, particularly in colorectal cancer (FIGS. 23A & 23B). As shown in FIGS. 24A-24C, colorectal cancer also showed a noticeable increase in IL-17A expression (FIG. 24A), IL-17F expression (FIG. 24B), and IL-17A/F expression (FIG. 24C). These findings may suggest a negative regulatory role of these cytokines in anti-tumor immunity, particularly in colorectal cancer.

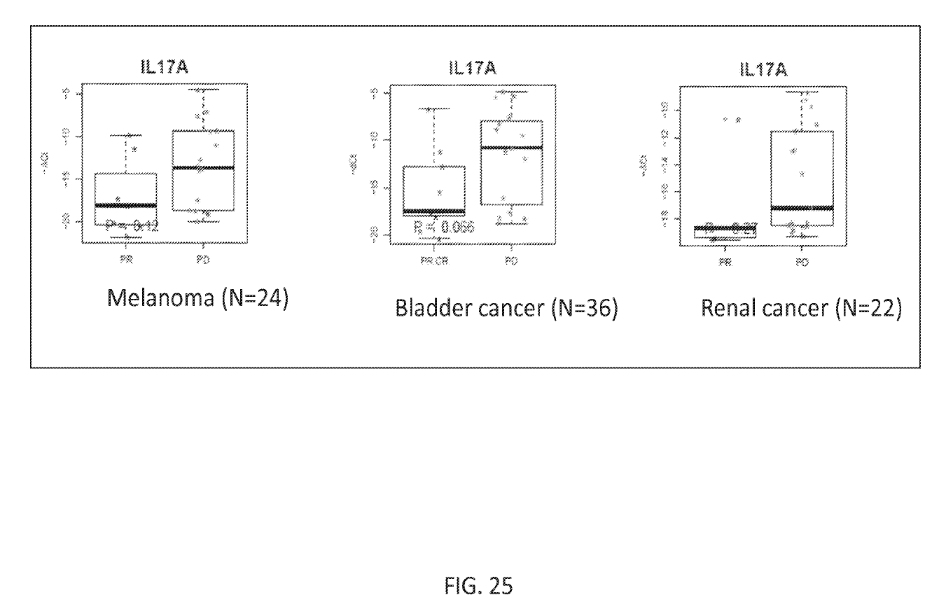

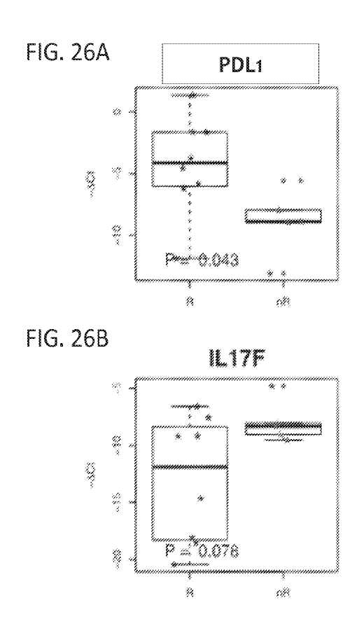

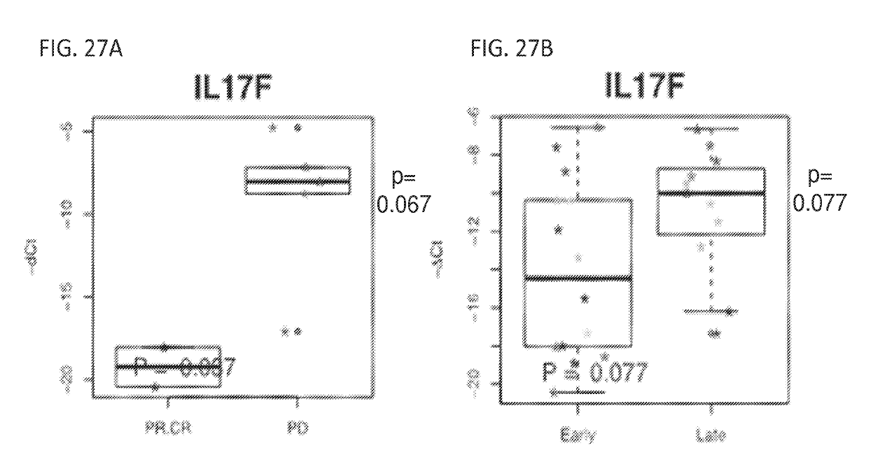

FIG. 25 further shows the correlation between increased IL-17A expression and progressive disease that was observed across melanoma, bladder cancer, and renal cancer indications. In addition, patients that were non-responsive to anti-PDL1 treatment for renal cell carcinoma showed a trend toward higher expression of IL-17F. This was evident in analyzing total patient data (FIGS. 26A & 26B). Even more clear were the observed trends for higher IL-17F expression patients with progressive disease on anti-PDL1 treatment, compared to a positive response on anti-PDL1 treatment (complete or partial response) (FIG. 27A), and for higher IL-17F expression in late responders to anti-PDL1 treatment (greater than 6 months) (FIG. 27B).

These data emphasize a correlation between higher IL-17 expression and a lack of responsiveness to anti-PDL1 treatment for various indications. For example, these data suggest that higher IL-17 expression correlates with a lack of responsiveness to anti-PDL1 treatment at least for bladder cancer, melanoma, and renal cell carcinoma. These data further suggest that increased expression of IL-17 and/or an IL-17 gene expression signature may also be detected in other types of cancer, notably colorectal cancer and ovarian cancer.

### Example 3: Localization of IL-17 Expression in Tumor Tissues

The previous Example demonstrates that IL-17 expression correlates with a lack of clinical response to anti-PDL1 treatment. In order to understand how IL-17 is expressed in different tumor tissues, IL-17 protein expression was visualized by immunohistochemical staining.

Immunohistochemical staining of various tissues for IL-17 protein was performed as described above. The detection reagent used was the anti-human IL-17A antibody AF-317. This reagent has been widely used to detect IL-17 in human tissues. For example, this antibody has been used to visualize IL-17A-expressing cells in cutaneous T-cell lymphoma (Fontao, L., et al. Br. J. Dermatol. 166:687-9 (2012)). Fontao et al. demonstrated that IL-17 staining correlated with CD3 staining in T cells, but in addition, other IL-17-positive cells that showed no CD3 expression were also identified. These cells were found to be morphologically similar to neutrophils and to stain for myeloperoxidase (MPO), suggesting that neutrophils may also show IL-17 expression.

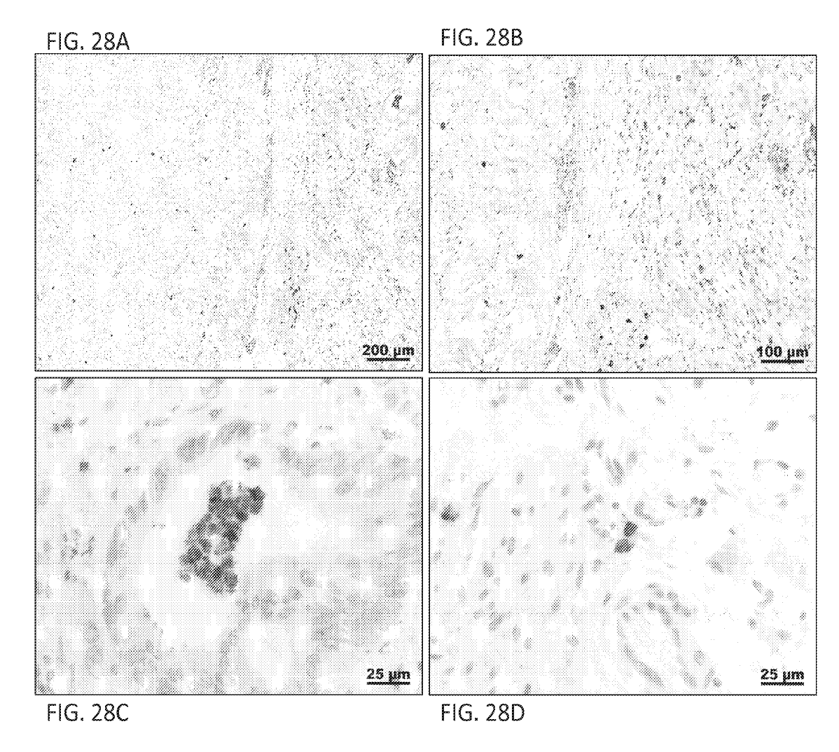

Non-small-cell lung cancer tissues were stained for IL-17A. As shown in FIGS. 28A-28D, IL-17A staining was observed in mononuclear cells consistent with lymphocytes and other small, round cells infiltrating the tumor stroma. Clusters of neutrophils with IL-17A expression were also identified (see, e.g., FIG. 28C).

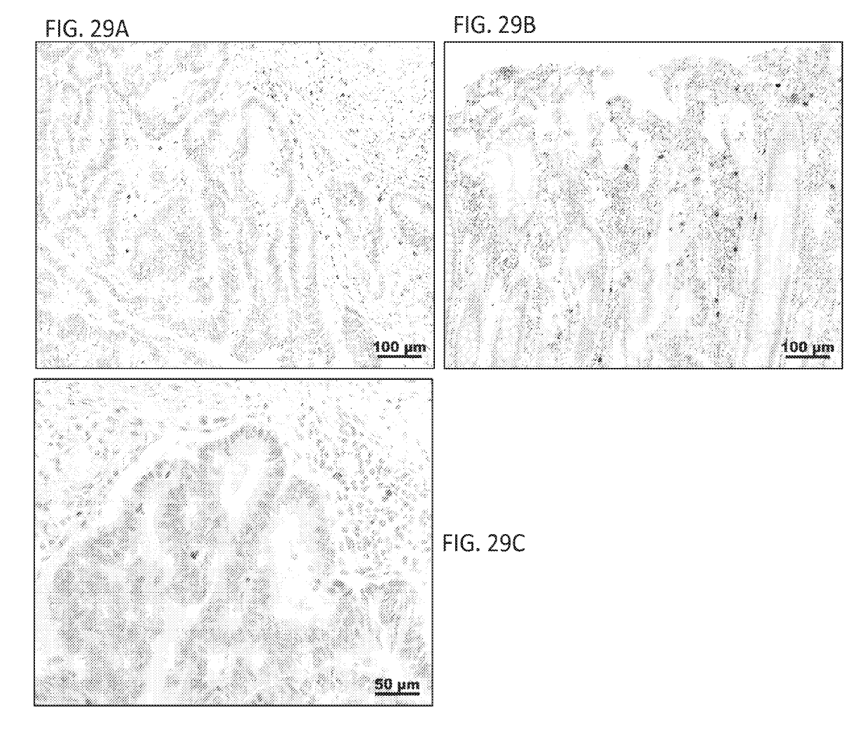

IL-17A staining was also assayed in colorectal cancer. FIGS. 29A-29C show IL-17A staining in colon adenocarcinoma samples. As in non-small-cell lung cancer, disseminated IL-17A-positive mononuclear cells were also observed in these samples, in addition to other cell types such as neutrophils that stained with weaker intensity.

Taken together, these immunohistochemical staining data clearly demonstrate the presence of IL-17A-positive mononuclear cells, as well as other IL-17A-positive cells (such as neutrophils), in multiple tumor types.

### Example 4: IL-17 Inducible Gene Expression in Mouse Tumor Models

IL-17 induces expression of numerous genes involved in pro-tumorigenic pathways. A panel of genes was selected as an IL-17 inducible gene signature to interrogate the contribution of the IL-17 pathway in mouse tumor models.

Syngeneic mice were inoculated subcutaneously with a panel of mouse tumor cell lines (i.e., cohorts of five to six mice were inoculated subcutaneously with a single tumor cell line). Once tumors were established and achieved a tumor volume of approximately 150-200 mm³, tumors were excised and processed for RNA for RNA-Seq analysis. Types of tumors included in the analysis were those derived from lung (NSCLC 082A and NSCLC 095A, TC-1), breast (4T1, EMT6.luc, JC), colon (51BLIM10, CT26, MC38), melanoma (Clone M-3, B16.F10, MEL-BR-1, SM1), and pancreas (KPR_3070, PAN 02 X1).

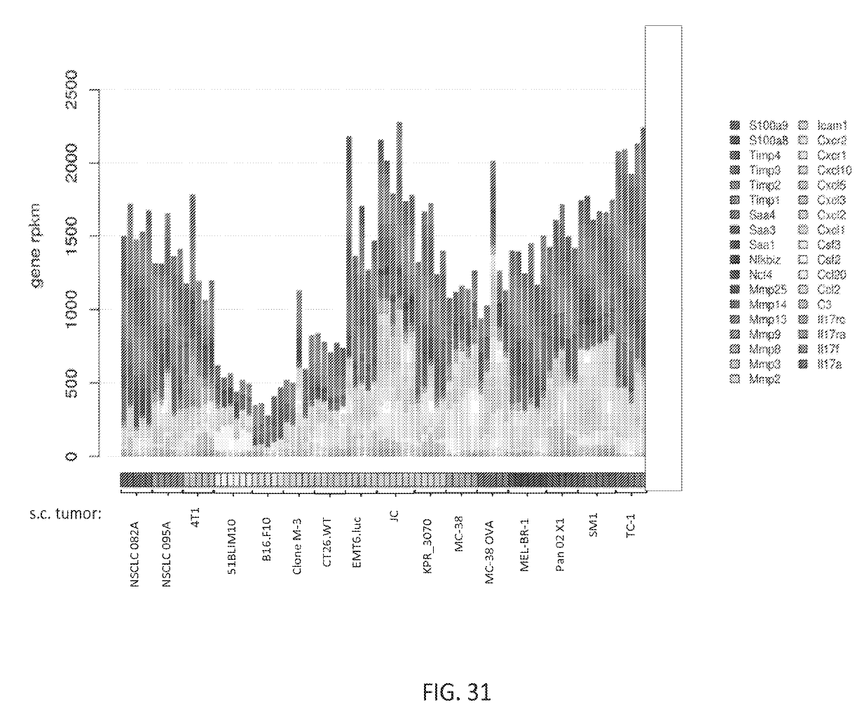

As shown in FIG. 31, the relative strength of the IL-17 inducible gene signature varied among tumors. For example, B16.F10 melanoma exhibited weak gene signature expression whereas another melanoma, SM1, had relatively high gene signature expression. Individual gene component expression within the IL-17 inducible gene signature was also variable. For example, EMT6 had relatively high contribution of S100A8 and S100A9 to the overall gene signature, whereas JC, another breast tumor, had relatively high MMP and TIMP contribution. As seen for preclinical tumor models, the IL-17 inducible gene signature may be predictive of cancers responsive to IL-17 binding antagonists as single agent or in combination with anti-PDL1. EMT6 displayed a relatively high IL-17 inducible gene signature and treatment with combination of both PD-1 axis binding antagonist and IL-17 binding antagonist dramatically reduced tumor size and growth rate in the EMT6 model, as shown in Example 1.

Next, for establishment of orthotopic lung tumors, syngeneic B6 (Cg)—Tyrc-2J/J mice were inoculated with 100,000 Lewis lung carcinoma (LLC) or B16.10 melanoma cells i.v. via tail vein injection. Lungs were harvested 24 days after inoculation with B16.F10 or at day 24 or day 29 post-inoculation for LLC.

For treatment with anti-IL17 antibodies, animals were inoculated with 1 million LLC cells in a volume of 100 microliters in HBSS via the tail vein. All inoculated mice were grouped based on an initial Micro CT scan. Mice with detectable tumors in lung on day 13 following tumor inoculation were randomized into two groups. One group was not treated. The second group was treated with anti-IL-17 cross-reactive antibody recognizing IL-17A and IL-17F (10 mg/kg) and anti-IL-17 antibody recognizing IL-17F (10 mg/kg) dosed IV for first dose, then dosed IP 3×/week. Treatment was initiated on day 14 post-inoculation. Naïve mice were not inoculated with LLC cells, nor were they treated with anti-IL-17 antibodies.

Measurements and weights were collected twice per week. Animals exhibiting weight loss of >15% were weighed daily and euthanized if they lost >20% body weight. Animals showing adverse clinical issues were observed more frequently, up to daily depending on severity, and euthanized if moribund. Mice were sacrificed on day 21 post-inoculation, after 1 week of anti-IL-17 treatment. Micro-CT scan was performed on day 20 to assess lung tissue volume. RNA was purified from tumor-bearing lungs and gene expression was determined using Fluidigm Dynamic Array Chip for Gene Expression and BioMark Real-Time PCR system.

Gene expression probes were as follows: housekeeping genes ACTB, GAPDH, RPL19; T cell markers CD4, CD8a; IL17 cytokines IL17A, IL17B, IL17C, IL17D, IL17F; IL17 receptors IL17RA, IL17RC; IL17 inducible genes C3, CCL2, CCL20, CSF2, CSF3, CXCL1, CXCL2, CXCL3, CXCL5, CXCL10, CXCR1, CXCR2, ICAM1, IL6, MMP1, MMP2, MMP3, MMP8, MMP9, MMP13, MMP14, MMP25, NCF4, NFKBIZ, S100A8, S100A9, SAA1.2 (i.e., a probe that detects both SAA1 and SAA2), SAA1, SAA3, SAA4, TIMP1, TIMP2, TIMP3, TIMP4.

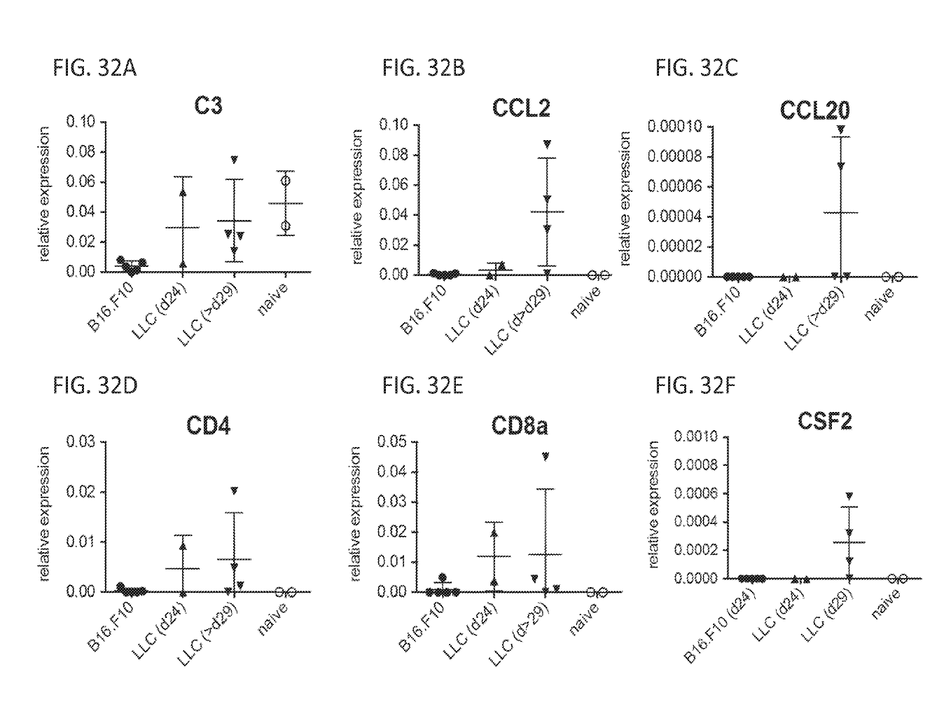

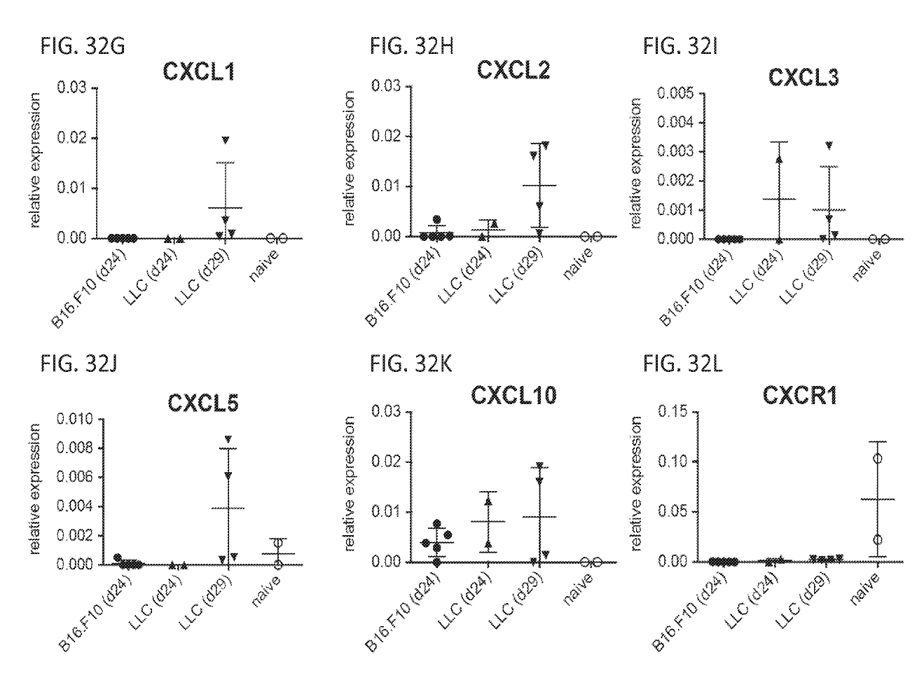

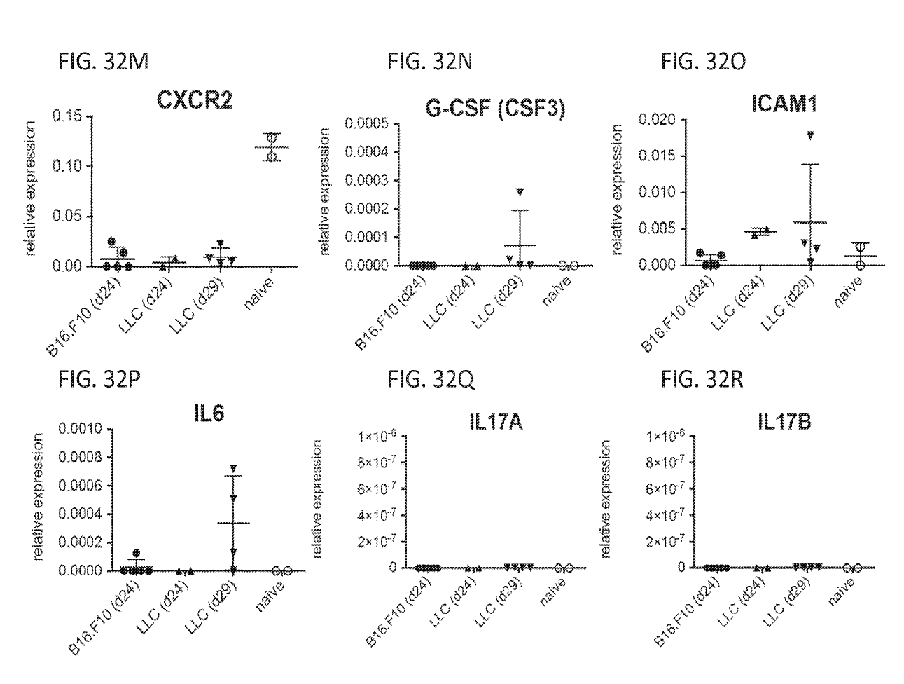

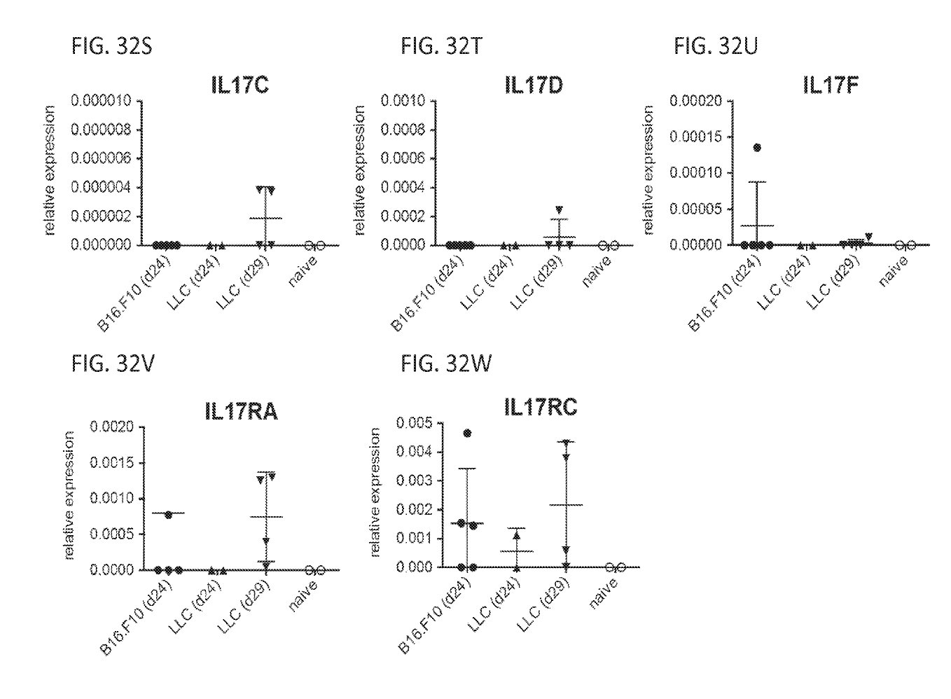

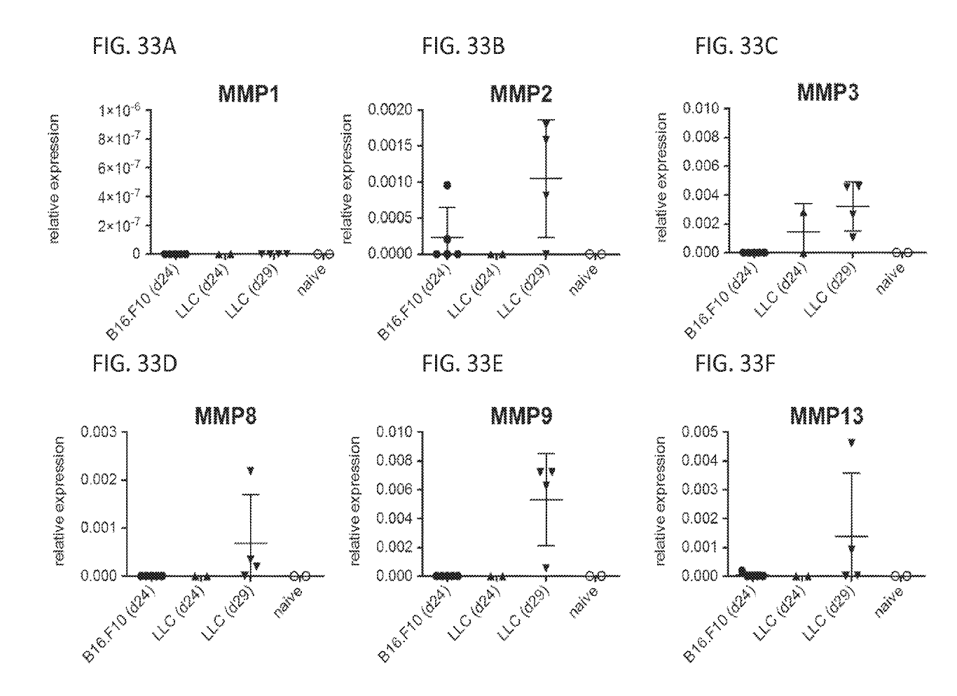

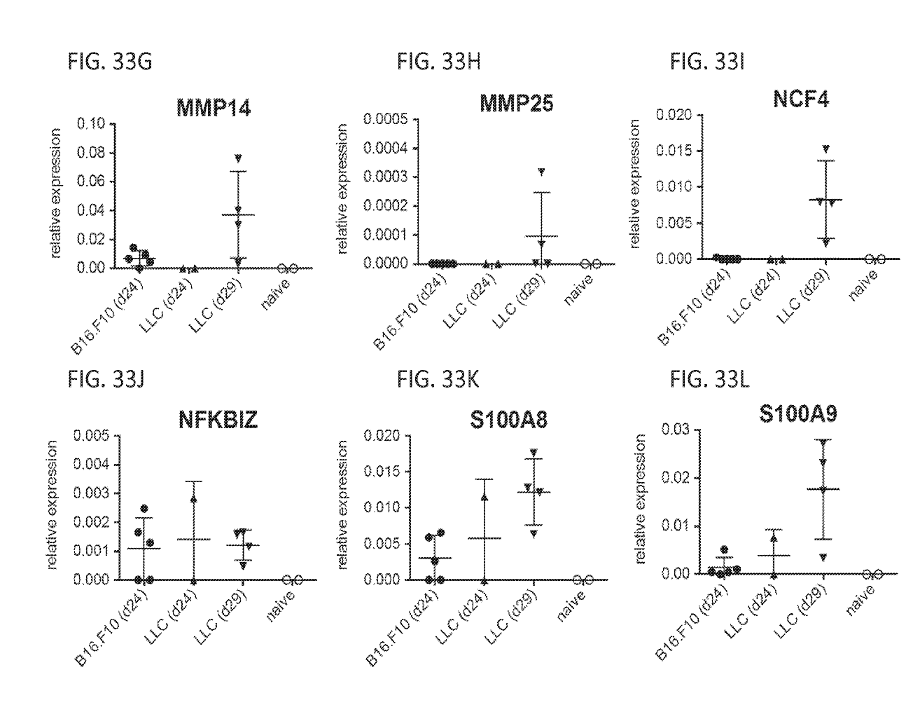

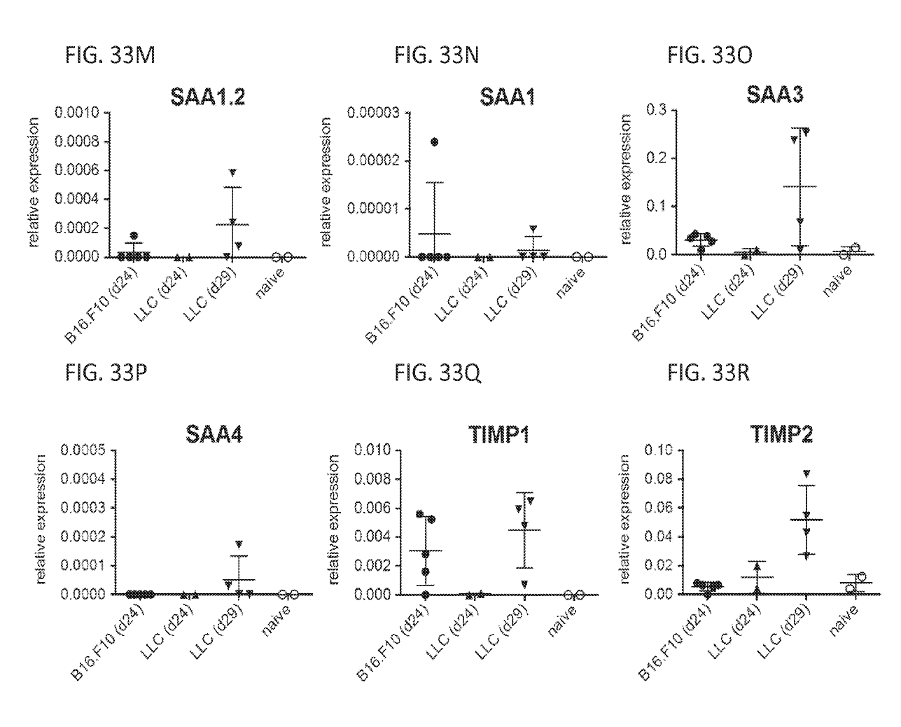

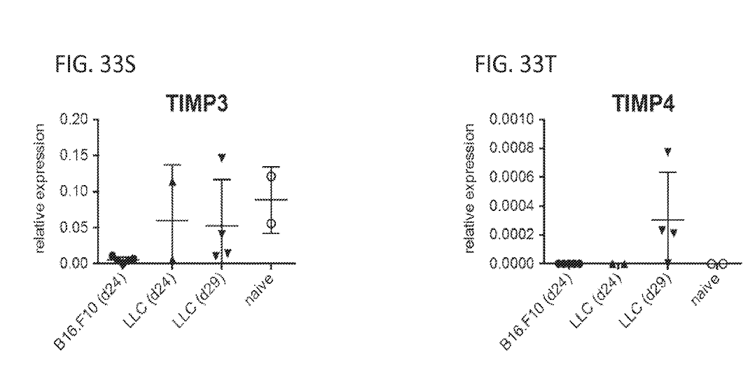

As shown in FIGS. 32A-32W and 33A-33T, expression of individual genes comprising the IL-17 inducible gene signature varied between orthotopic B16.F10 and LLC lung tumors. Overall, LLC lung tumors had higher expression of IL-17 inducible gene signature components than B16.F10. Furthermore, relative expression levels of these genes increased in day 29 LLC lung tumors compared to day 24, indicating that IL-17 gene signature becomes more pronounced as the tumor progresses. These data demonstrate that IL-17 gene signature may be used to identify cancers with IL-17 pathway involvement, and therefore be amenable to IL-17 binding antagonists.

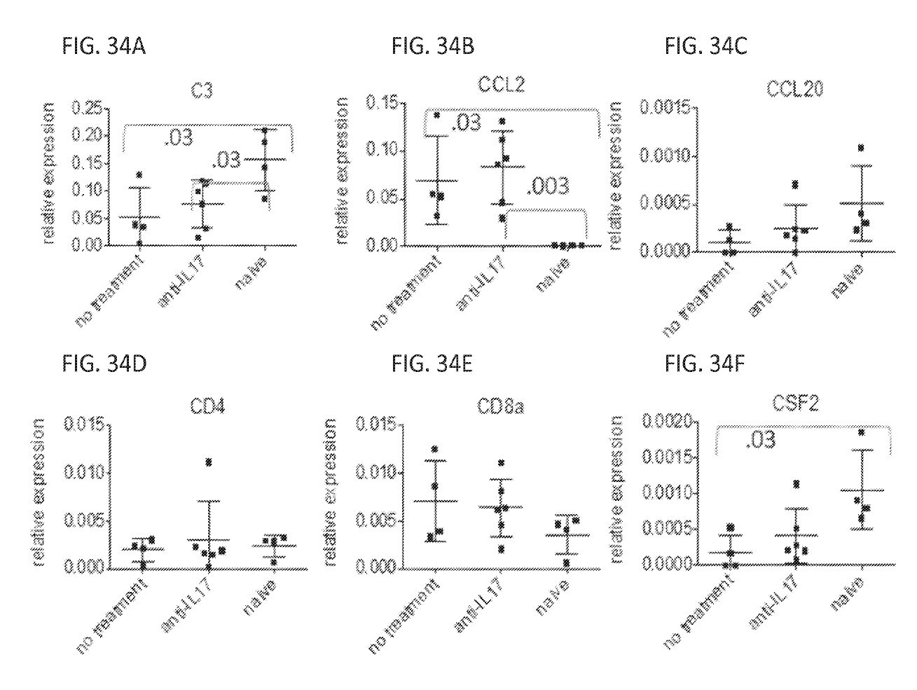

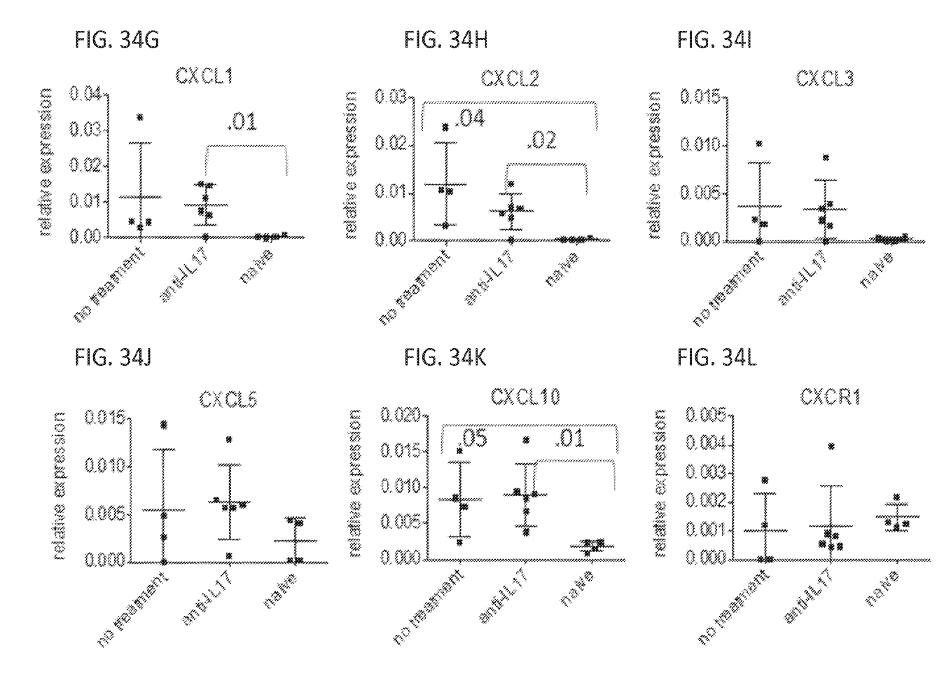

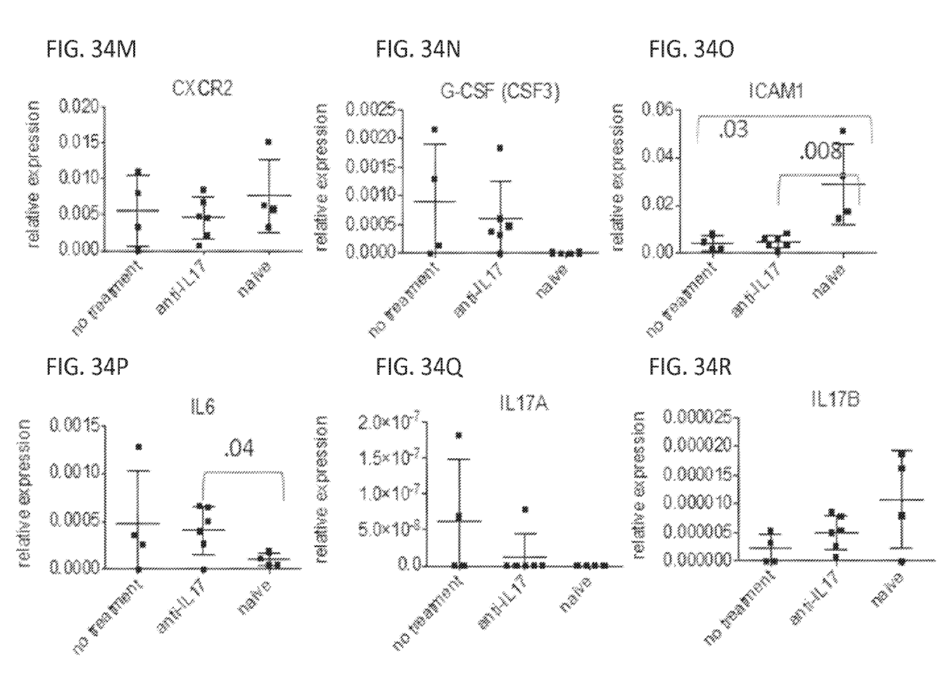

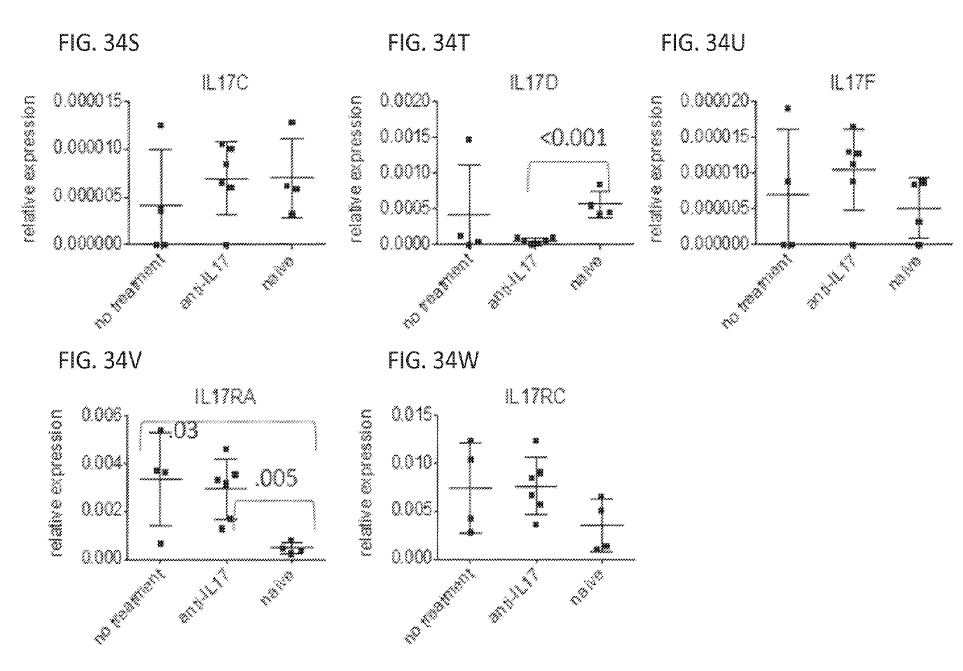

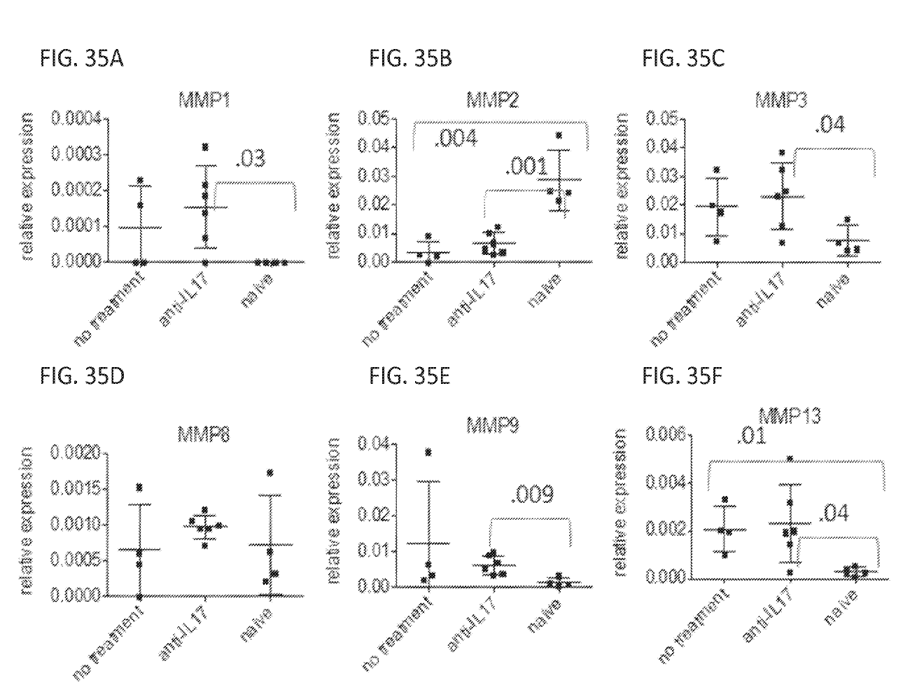

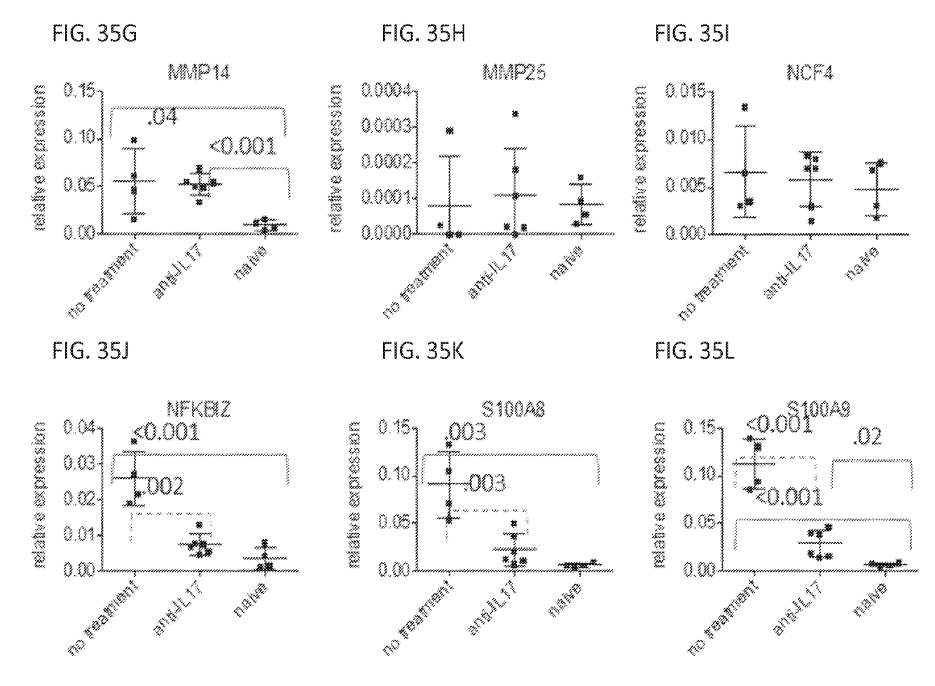

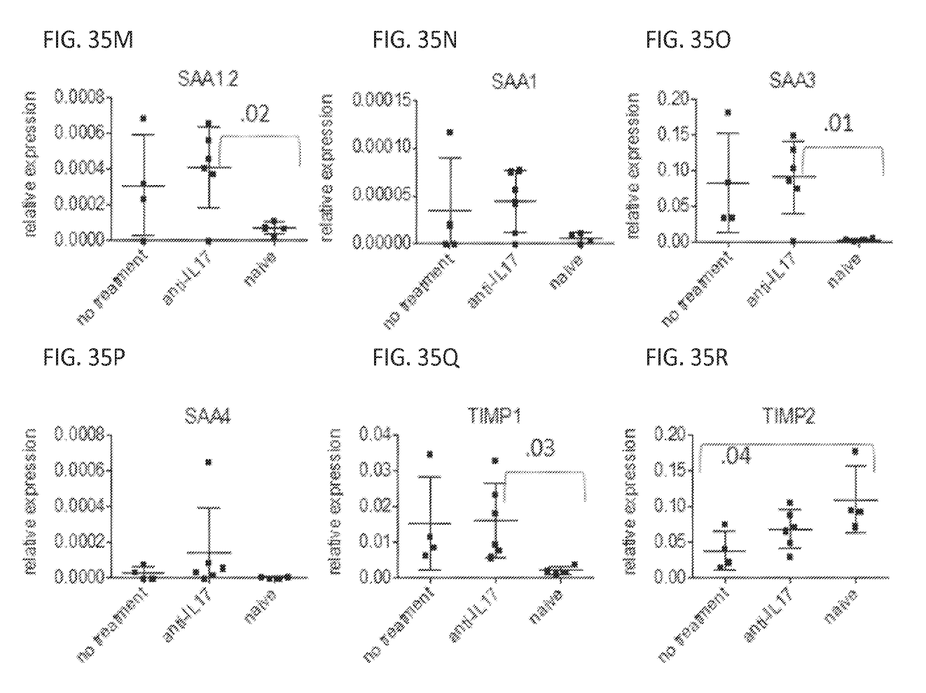

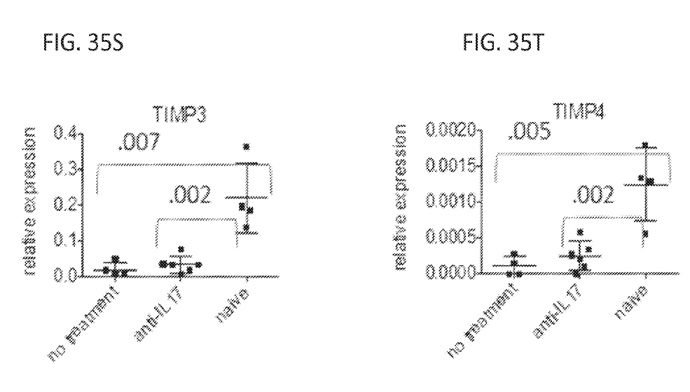

FIGS. 34A-34W and 35A-35T show the effects of anti-IL-17 treatment on expression of IL-17 inducible genes in orthotopic LLC lung tumors. After 1 week treatment, inhibition of gene expression was observed for some genes, notably NFKBIZ (FIG. 35J), S100A8 (FIG. 35K) and S100A9 (FIG. 35L). This indicates that anti-IL-17 antibody treatment can modulate expression of genes that contribute to tumor progression. Monitoring changes in IL-17 gene signature expression may serve as a biomarker tool for assessing effects of IL-17 binding antagonists in an oncology setting.

All patents, patent applications, documents, and articles cited herein are incorporated herein by reference in their entireties.

1. A method for treating or delaying progression of cancer in an individual comprising administering to the individual an effective amount of a PD-1 axis binding antagonist and an IL-17 binding antagonist.

2. A method for identifying an individual with cancer for treatment with a PD-1 axis binding antagonist and an IL-17 binding antagonist, the method comprising:
(a) detecting expression of IL-17 in a biopsy sample obtained from the cancer in the individual; and
(b) if the biopsy sample shows expression of IL-17, or if the biopsy sample shows increased expression of IL-17 as compared to a reference or a reference sample, administering to the individual an effective amount of a PD-1 axis binding antagonist and an IL-17 binding antagonist.

3. A method for identifying an individual with cancer for treatment with a PD-1 axis binding antagonist and an IL-17 binding antagonist, the method comprising:
(a) detecting expression of one or more genes selected from the group consisting of CD4, CD8a, IL17A, IL17B, IL17C, IL17D, IL17F, IL17RA, IL17RC, C3, CCL2, CCL20, CSF2, CSF3, CXCL1, CXCL2, CXCL3, CXCL5, CXCL10, CXCR1, CXCR2, ICAM1, IL6, IL8, MMP1, MMP2, MMP3, MMP8, MMP9, MMP13, MMP14, MMP25, NCF4, NFKBIZ, S100A8, S100A9, SAA2, SAA1, SAA3, SAA4, TIMP1, TIMP2, TIMP3, and TIMP4 in a biopsy sample obtained from the cancer in the individual; and
(b) if the biopsy sample shows expression of the one or more genes selected from the group consisting of CD4, CD8a, IL17A, IL17B, IL17C, IL17D, IL17F, IL17RA, IL17RC, C3, CCL2, CCL20, CSF2, CSF3, CXCL1, CXCL2, CXCL3, CXCL5, CXCL10, CXCR1, CXCR2, ICAM1, IL6, IL8, MMP1, MMP2, MMP3, MMP8, MMP9, MMP13, MMP14, MMP25, NCF4, NFKBIZ, S100A8, S100A9, SAA2, SAA1, SAA3, SAA4, TIMP1, TIMP2, TIMP3, and TIMP4, or if the biopsy sample shows increased expression of the one or more genes selected from the group consisting of CD4, CD8a, IL17A, IL17B, IL17C, IL17D, IL17F, IL17RA, IL17RC, C3, CCL2, CCL20, CSF2, CSF3, CXCL1, CXCL2, CXCL3, CXCL5, CXCL10, CXCR1, CXCR2, ICAM1, IL6, IL8, MMP1, MMP2, MMP3, MMP8, MMP9, MMP13, MMP14, MMP25, NCF4, NFKBIZ, S100A8, S100A9, SAA2, SAA1, SAA3, SAA4, TIMP1, TIMP2, TIMP3, and TIMP4 as compared to a reference or a reference sample, administering to the individual an effective amount of a PD-1 axis binding antagonist and an IL-17 binding antagonist.

4. The method of claim 1, wherein the PD-1 axis binding antagonist is selected from the group consisting of a PD-1 binding antagonist, a PDL1 binding antagonist and a PDL2 binding antagonist.

5. The method of claim 4, wherein the PD-1 axis binding antagonist is a PD-1 binding antagonist.

6. The method of claim 5, wherein the PD-1 binding antagonist inhibits the binding of PD-1 to its ligand binding partners.

7. The method of claim 6, wherein the PD-1 binding antagonist inhibits the binding of PD-1 to PDL1, inhibits the binding of PD-1 to PDL2, or inhibits the binding of PD-1 to both PDL1 and PDL2.

8. (canceled)

9. (canceled)

10. The method of claim 6, wherein the PD-1 binding antagonist is an antibody.

11. The method of claim 10, wherein the PD-1 binding antagonist is nivolumab, pembrolizumab, MEDI-0680, PDR001, REGN2810, BGB-108, or BGB-A317.

12-14. (canceled)

15. The method of claim 6, wherein the PD-1 binding antagonist is AMP-224.

16. The method of claim 4, wherein the PD-1 axis binding antagonist is a PDL1 binding antagonist.

17. The method of claim 16, wherein the PDL1 binding antagonist inhibits the binding of PDL1 to PD-1, inhibits the binding of PDL1 to B7-1, or inhibits the binding of PDL1 to both PD-1 and B7-1.

18. (canceled)

19. (canceled)

20. The method of claim 16, wherein the PDL1 binding antagonist is an anti-PDL1 antibody.

21. The method of claim 20, wherein the anti-PDL1 antibody is a monoclonal antibody.

22. The method of claim 20, wherein the anti-PDL1 antibody is an antibody fragment selected from the group consisting of Fab, Fab′-SH, Fv, scFv, and (Fab′)₂ fragments.

23. The method of claim 20, wherein the anti-PDL1 antibody is a humanized antibody or a human antibody.

24. The method of claim 16, wherein the PDL1 binding antagonist is selected from the group consisting of: YW243.55.S70, MPDL3280A, MDX-1105, MEDI4736, and avelumab.

25. The method of claim 20, wherein the antibody comprises a heavy chain comprising HVR-H1 sequence of SEQ ID NO:15, HVR-H2 sequence of SEQ ID NO:16, and HVR-H3 sequence of SEQ ID NO:3; and a light chain comprising HVR-L1 sequence of SEQ ID NO:17, HVR-L2 sequence of SEQ ID NO:18, and HVR-L3 sequence of SEQ ID NO:19.

26. The method of claim 20, wherein the antibody comprises a heavy chain variable region comprising the amino acid sequence of SEQ ID NO:24 or SEQ ID NO:28 and a light chain variable region comprising the amino acid sequence of SEQ ID NO:21.

27. The method of claim 4, wherein the PD-1 axis binding antagonist is a PDL2 binding antagonist.

28. The method of claim 27, wherein the PDL2 binding antagonist is an antibody.

29. The method of claim 27, wherein the PDL2 binding antagonist is an immunoadhesin.

30. The method of claim 1, wherein the IL-17 binding antagonist inhibits the binding of IL-17 to the IL-17 receptor.

31. The method of claim 30, wherein the IL-17 binding antagonist is an antibody.

32. The method of claim 31, wherein the IL-17 binding antagonist is a monoclonal antibody.

33. The method of claim 31, wherein the IL-17 binding antagonist is an antibody fragment selected from the group consisting of Fab, Fab′-SH, Fv, scFv, and (Fab′)₂ fragments.

34. The method of claim 31, wherein the IL-17 binding antagonist is a humanized antibody or a human antibody.

35. The method of claim 31, wherein the antibody comprises a heavy chain comprising CDR-H1 sequence of SEQ ID NO:32, CDR-H2 sequence of SEQ ID NO:33, and CDR-H3 sequence of SEQ ID NO:34; and a light chain comprising CDR-L1 sequence of SEQ ID NO:35, CDR-L2 sequence of SEQ ID NO:36, and CDR-L3 sequence of SEQ ID NO:37.

36. The method of claim 31, wherein the antibody comprises a heavy chain variable region comprising the amino acid sequence of SEQ ID NO:30 and a light chain variable region comprising the amino acid sequence of SEQ ID NO:31.

37. The method of claim 31, wherein the antibody comprises a heavy chain comprising CDR-H1 sequence of SEQ ID NO:40, CDR-H2 sequence of SEQ ID NO:41, and CDR-H3 sequence of SEQ ID NO:42; and a light chain comprising CDR-L1 sequence of SEQ ID NO:43, CDR-L2 sequence of SEQ ID NO:44, and CDR-L3 sequence of SEQ ID NO:45.

38. The method of claim 31, wherein the antibody comprises a heavy chain variable region comprising the amino acid sequence of SEQ ID NO:38 and a light chain variable region comprising the amino acid sequence of SEQ ID NO:39.

39. The method of claim 31, wherein the antibody comprises a heavy chain comprising CDR-H1 sequence of SEQ ID NO:48, CDR-H2 sequence of SEQ ID NO:49, and CDR-H3 sequence of SEQ ID NO:50; and a light chain comprising CDR-L1 sequence of SEQ ID NO:51, CDR-L2 sequence of SEQ ID NO:52, and CDR-L3 sequence of SEQ ID NO:53.

40. The method of claim 31, wherein the antibody comprises a heavy chain variable region comprising the amino acid sequence of SEQ ID NO:46 and a light chain variable region comprising the amino acid sequence of SEQ ID NO:47.

41. The method of claim 31, wherein the antibody comprises a heavy chain comprising CDR-H1 sequence of SEQ ID NO:56, CDR-H2 sequence of SEQ ID NO:57, and CDR-H3 sequence of SEQ ID NO:58; and a light chain comprising CDR-L1 sequence of SEQ ID NO:59, CDR-L2 sequence of SEQ ID NO:60, and CDR-L3 sequence of SEQ ID NO:61.

42. The method of claim 31, wherein the antibody comprises a heavy chain variable region comprising the amino acid sequence of SEQ ID NO:54 and a light chain variable region comprising the amino acid sequence of SEQ ID NO:55.

43. The method of claim 31, wherein the IL-17 binding antagonist is an anti-IL-17 antibody.

44. The method of claim 43, wherein the anti-IL-17 antibody specifically binds to IL-17A, specifically binds to IL-17F, or specifically binds to IL-17A and IL-17F.

45. (canceled)

46. (canceled)

47. The method of claim 43, wherein the anti-IL-17 antibody is ixekizumab, bimekizumab, or secukinumab.

48. (canceled)

49. (canceled)

50. The method of claim 31, wherein the IL-17 binding antagonist is an anti-IL-17 receptor antibody.

51. The method of claim 50, wherein the anti-IL-17 receptor antibody is brodalumab.

52. The method of claim 30, wherein the IL-17 binding antagonist is a soluble polypeptide comprising at least one exon from an IL-17 receptor.

53. The method of claim 52, wherein the soluble polypeptide comprises at least one exon from IL-17RA and at least one exon from IL-17RC.

54. The method of claim 1, wherein a biopsy sample obtained from the cancer of the individual shows expression of IL-17.

55. The method of claim 54, wherein the expression of IL-17 is expression of IL-17 mRNA.

56. The method of claim 54, wherein the expression of IL-17 is expression of IL-17 protein.

57. The method of claim 54, wherein the biopsy sample obtained from the cancer shows elevated expression of IL-17 as compared to a reference sample.

58. The method of claim 1, wherein a biopsy sample obtained from the cancer of the individual shows expression of one or more genes selected from the group consisting of CD4, CD8a, IL17A, IL17B, IL17C, IL17D, IL17F, IL17RA, IL17RC, C3, CCL2, CCL20, CSF2, CSF3, CXCL1, CXCL2, CXCL3, CXCL5, CXCL10, CXCR1, CXCR2, ICAM1, IL6, IL8, MMP1, MMP2, MMP3, MMP8, MMP9, MMP13, MMP14, MMP25, NCF4, NFKBIZ, S100A8, S100A9, SAA2, SAA1, SAA3, SAA4, TIMP1, TIMP2, TIMP3, and TIMP4.

59. The method of claim 57, wherein the biopsy sample obtained from the cancer shows elevated expression of one or more genes selected from the group consisting of CD4, CD8a, IL17A, IL17B, IL17C, IL17D, IL17F, IL17RA, IL17RC, C3, CCL2, CCL20, CSF2, CSF3, CXCL1, CXCL2, CXCL3, CXCL5, CXCL10, CXCR1, CXCR2, ICAM1, IL6, IL8, MMP1, MMP2, MMP3, MMP8, MMP9, MMP13, MMP14, MMP25, NCF4, NFKBIZ, S100A8, S100A9, SAA2, SAA1, SAA3, SAA4, TIMP1, TIMP2, TIMP3, and TIMP4 as compared to a reference sample.

60. The method of claim 1, wherein a biopsy sample obtained from the cancer of the individual shows expression of one or more genes selected from the group consisting of NFKBIZ, S100A8, and S100A9.

61. The method of claim 60, wherein the biopsy sample obtained from the cancer shows elevated expression of one or more genes selected from the group consisting of NFKBIZ, S100A8, and S100A9 as compared to a reference sample.

62. The method of claim 1, wherein the cancer is selected from the group consisting of renal cell carcinoma, bladder cancer, non-small-cell lung cancer, squamous non-small-cell lung cancer, non-squamous non-small-cell lung cancer, colorectal cancer, melanoma, ovarian cancer, breast cancer, hormone receptor-positive breast cancer, HER2-positive breast cancer, and triple-negative breast cancer.

63. The method of claim 1, wherein the treatment results in a sustained response in the individual after cessation of the treatment.

64. The method of claim 1, wherein the IL-17 binding antagonist or the PD-1 axis binding antagonist is administered continuously or intermittently.

65. The method of claim 1, wherein the IL-17 binding antagonist is administered before the PD-1 axis binding antagonist.

66. The method of claim 1, wherein the IL-17 binding antagonist is administered simultaneous with the PD-1 axis binding antagonist.

67. The method of claim 66, wherein the IL-17 binding antagonist and the PD-1 axis binding antagonist are formulated in the same composition.

68. The method of claim 1, wherein the IL-17 binding antagonist is administered after the PD-1 axis binding antagonist.

69. A method of enhancing immune function in an individual having cancer comprising administering an effective amount of a combination of a PD-1 axis binding antagonist and an IL-17 binding antagonist.

70. The method of claim 1, wherein the PD-1 axis binding antagonist or the IL-17 binding antagonist is administered intravenously, intramuscularly, subcutaneously, topically, orally, transdermally, intraperitoneally, intraorbitally, by implantation, by inhalation, intrathecally, intraventricularly, or intranasally.

71. A kit comprising a PD-1 axis binding antagonist and a package insert comprising instructions for using the PD-1 axis binding antagonist in combination with an IL-17 binding antagonist to treat or delay progression of cancer in an individual.

72. (canceled)

73. (canceled)

74. A kit comprising an IL-17 binding antagonist and a package insert comprising instructions for using the IL-17 binding antagonist in combination with a PD-1 axis binding antagonist to treat or delay progression of cancer in an individual.

75-78. (canceled)
